SEQUENCE LISTING (119 sequences)

SEQ ID NO: 1 - protein, 117 aa, synthetic construct
  source 1..117: note = Human CD47-C33G
QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 2 - protein, 117 aa, synthetic construct
  source 1..117: note = 108VH
QMQLQQSGPQLVRPGASVKISCKTSGYSFTHHWIHWMKQRPGQGLEWIGMIDPSDSETRLSQKFKDKATLTVDASSSTAYMQLNSPTSEDSALYFCARLGRYYFDYWGQGTTLTVSS

SEQ ID NO: 3 - protein, 107 aa, synthetic construct
  source 1..107: note = 108VL
NIVMTQSPRSMSMSVGERVTLSCKASENVGTYISWYQQKPDQSPKLLIYGASNRYTGVPDRFTGSGSGTDFTLTISTVQAEDLADYHCGESYGHLYTFGGGTKLEIK

SEQ ID NO: 4 - protein, 117 aa, synthetic construct
  source 1..117: note = 108VH4.M4
EVQLVQSGAEVKKPGSSVKVSCKASGYSFTHHWIHWVRQAPGQGLEWMGMIDASDSETRLSQKFKDRVTITADKSTSTAYMELSSLRSEDTAVYYCARLGRYYFDYWGQGTTVTVSS

SEQ ID NO: 5 - protein, 107 aa, synthetic construct
  source 1..107: note = 108VL1.M1
EIVLTQSPATLSLSPGERATLSCRASENVGTYISWYQQKPGQAPRLLIYGASNRYTGIPARFSGSGSGTDFTLTISSLEPEDFAVYYCGESYGHLYTFGGGTKVEIK

SEQ ID NO: 6 - protein, 117 aa, synthetic construct
  source 1..117: note = 108VH5
EVQLVQSGAEVKKPGASVKVSCKASGYSFTHHWIHWVRQAPGQGLEWMGMIDPSDSETRLSQKFKDRVTMTRDTSTSTVYMELSSLRSEDTAVYYCARLGRYYFDYWGQGTLVTVSS

SEQ ID NO: 7 - protein, 107 aa, synthetic construct
  source 1..107: note = 108VL1.M5
EIVLTQSPATLSLSPGERATLSCRASQSVSSYLAWYQQKPGQAPRLLIYGASNRATGIPARFSGSGSGTDFTLTISSLEPEDFAVYYCGESYGHLYTFGGGTKVEIK

SEQ ID NO: 8 - protein, 117 aa, synthetic construct
  source 1..117: note = 108VH5.M5
EVQLVQSGAEVKKPGASVKVSCKASGYTFTHHWIHWVRQAPGQGLEWMGMIDASDSETRLSQKFKDRVTMTRDTSTSTVYMELSSLRSEDTAVYYCARLGRYYFDYWGQGTLVTVSS

SEQ ID NO: 9 - protein, 117 aa, synthetic construct
  source 1..117: note = 108VH5.M6
EVQLVQSGAEVKKPGASVKVSCKASGYTFTHHYMHWVRQAPGQGLEWMGMIDASDSETRLSQKFKDRVTMTRDTSTSTVYMELSSLRSEDTAVYYCARLGRYYFDYWGQGTLVTVSS

SEQ ID NO: 10 - protein, 117 aa, synthetic construct
  source 1..117: note = 108VH5.M8
EVQLVQSGAEVKKPGASVKVSCKASGYSFTHHWIHWVRQAPGQGLEWMGMIDASDSETRYAQKFKDRVTMTRDTSTSTVYMELSSLRSEDTAVYYCARLGRYYFDYWGQGTLVTVSS

SEQ ID NO: 11 - protein, 117 aa, synthetic construct
  source 1..117: note = 108VH5.M9
EVQLVQSGAEVKKPGASVKVSCKASGYSFTHHWIHWVRQAPGQGLEWMGMIDASDSETRLSQKFQGRVTMTRDTSTSTVYMELSSLRSEDTAVYYCARLGRYYFDYWGQGTLVTVSS

SEQ ID NO: 12 - protein, 117 aa, synthetic construct
  source 1..117: note = 108VH5.M10
EVQLVQSGAEVKKPGASVKVSCKASGYTFTHHWIHWVRQAPGQGLEWMGMIDASDSETRYAQKFQGRVTMTRDTSTSTVYMELSSLRSEDTAVYYCARLGRYYFDYWGQGTLVTVSS

SEQ ID NO: 13 - protein, 117 aa, synthetic construct
  source 1..117: note = 108VH5.M11
EVQLVQSGAEVKKPGASVKVSCKASGYTFTHHYIHWVRQAPGQGLEWMGMIDASDSETRYAQKFQGRVTMTRDTSTSTVYMELSSLRSEDTAVYYCARLGRYYFDYWGQGTLVTVSS

SEQ ID NO: 14 - protein, 117 aa, synthetic construct
  source 1..117: note = 108VH5.M12
EVQLVQSGAEVKKPGASVKVSCKASGYTFTHHWMHWVRQAPGQGLEWMGMIDASDSETRYAQKFQGRVTMTRDTSTSTVYMELSSLRSEDTAVYYCARLGRYYFDYWGQGTLVTVSS

SEQ ID NO: 15 - protein, 117 aa, synthetic construct
  source 1..117: note = 108VH5.M14
EVQLVQSGAEVKKPGASVKVSCKASGYTFTHHWIHWVRQAPGQGLEWMGMIDASDGETRYAQKFQGRVTMTRDTSTSTVYMELSSLRSEDTAVYYCARLGRYYFDYWGQGTLVTVSS

SEQ ID NO: 16 - protein, 117 aa, synthetic construct
  source 1..117: note = 108VH5.M15
EVQLVQSGAEVKKPGASVKVSCKASGYTFTHHWIHWVRQAPGQGLEWMGMIDASDSSTRYAQKFQGRVTMTRDTSTSTVYMELSSLRSEDTAVYYCARLGRYYFDYWGQGTLVTVSS

SEQ ID NO: 17 - protein, 117 aa, synthetic construct
  source 1..117: note = 108VH5.M16
EVQLVQSGAEVKKPGASVKVSCKASGYTFTHHWMHWVRQAPGQGLEWMGMIDASDGETRYAQKFQGRVTMTRDTSTSTVYMELSSLRSEDTAVYYCARLGRYYFDYWGQGTLVTVSS

SEQ ID NO: 18 - protein, 117 aa, synthetic construct
  source 1..117: note = 108VH5.M17
EVQLVQSGAEVKKPGASVKVSCKASGYTFTHHWMHWVRQAPGQGLEWMGMIDASEGETRYAQKFQGRVTMTRDTSTSTVYMELSSLRSEDTAVYYCARLGRYYFDYWGQGTLVTVSS

SEQ ID NO: 19 - protein, 117 aa, synthetic construct
  source 1..117: note = 108VH5.M18
EVQLVQSGAEVKKPGASVKVSCKASGYTFTHHWMHWVRQAPGQGLEWMGMIDASESETRYAQKFQGRVTMTRDTSTSTVYMELSSLRSEDTAVYYCARLGRYYFDYWGQGTLVTVSS

SEQ ID NO: 20 - protein, 117 aa, synthetic construct
  source 1..117: note = 108VH5.M20
EVQLVQSGAEVKKPGASVKVSCKASGYTFTHHWMHWVRQAPGQGLEWMGMIDASDAETRYAQKFQGRVTMTRDTSTSTVYMELSSLRSEDTAVYYCARLGRYYFDYWGQGTLVTVSS

SEQ ID NO: 21 - protein, 117 aa, synthetic construct
  source 1..117: note = 108VH5.M21
EVQLVQSGAEVKKPGASVKVSCKASGYTFTHHWMHWVRQAPGQGLEWMGMIDASDFETRYAQKFQGRVTMTRDTSTSTVYMELSSLRSEDTAVYYCARLGRYYFDYWGQGTLVTVSS

SEQ ID NO: 22 - protein, 117 aa, synthetic construct
  source 1..117: note = 108VH5.M22
EVQLVQSGAEVKKPGASVKVSCKASGYTFTHHWMHWVRQAPGQGLEWMGMIDASDIETRYAQKFQGRVTMTRDTSTSTVYMELSSLRSEDTAVYYCARLGRYYFDYWGQGTLVTVSS

SEQ ID NO: 23 - protein, 117 aa, synthetic construct
  source 1..117: note = 108VH5.M23
EVQLVQSGAEVKKPGASVKVSCKASGYTFTHHWMHWVRQAPGQGLEWMGMIDASDKETRYAQKFQGRVTMTRDTSTSTVYMELSSLRSEDTAVYYCARLGRYYFDYWGQGTLVTVSS

SEQ ID NO: 24 - protein, 117 aa, synthetic construct
  source 1..117: note = 108VH5.M24
EVQLVQSGAEVKKPGASVKVSCKASGYTFTHHWMHWVRQAPGQGLEWMGMIDASDLETRYAQKFQGRVTMTRDTSTSTVYMELSSLRSEDTAVYYCARLGRYYFDYWGQGTLVTVSS

SEQ ID NO: 25 - protein, 117 aa, synthetic construct
  source 1..117: note = 108VH5.M25
EVQLVQSGAEVKKPGASVKVSCKASGYTFTHHWMHWVRQAPGQGLEWMGMIDASDRETRYAQKFQGRVTMTRDTSTSTVYMELSSLRSEDTAVYYCARLGRYYFDYWGQGTLVTVSS

SEQ ID NO: 26 - protein, 117 aa, synthetic construct
  source 1..117: note = 108VH5.M26
EVQLVQSGAEVKKPGASVKVSCKASGYTFTHHWMHWVRQAPGQGLEWMGMIDASDVETRYAQKFQGRVTMTRDTSTSTVYMELSSLRSEDTAVYYCARLGRYYFDYWGQGTLVTVSS

SEQ ID NO: 27 - protein, 117 aa, synthetic construct
  source 1..117: note = 108VH5.M27
EVQLVQSGAEVKKPGASVKVSCKASGYTFTHHWMHWVRQAPGQGLEWMGMIDASDYETRYAQKFQGRVTMTRDTSTSTVYMELSSLRSEDTAVYYCARLGRYYFDYWGQGTLVTVSS

SEQ ID NO: 28 - protein, 327 aa, synthetic construct
  source 1..327: note = IgG4 heavy chain constant region with S228P mutation
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 107 aa, synthetic construct [sequence omitted]

SEQ ID NO: 30 - protein, 107 aa, synthetic construct
  source 1..107: note = 108VL10.M1
DIQMTQSPSSLSASVGDRVTITCRASENVGTYISWYQQKPGKAPKLLIYGASNRYTGVPSRFSGSRSGTDFTLTISSLQPEDFATYYCGESYGHLYTFGQGTKVEIK

SEQ ID NO: 31 - protein, 117 aa, synthetic construct
  source 1..117: note = 108VH10
EVQLVESGGGLVQPGGSLRLSCAASGYSFTHHWIHWVRQAPGKGLEWVAMIDPSDSETRLSQKFKDRFTISADTSKNTAYLQMNSLRAEDTAVYYCSRLGRYYFDYWGQGTLVTVSS

SEQ ID NO: 32 - protein, 107 aa, synthetic construct
  source 1..107: note = 108VL10.Ma
DIQMTQSPSSLSASVGDRVTITCRASQSISSYLNWYQQKPGKAPKLLIYGASNRYTGVPSRFSGSRSGTDFTLTISSLQPEDFATYYCGESYGHLYTFGQGTKVEIK

SEQ ID NO: 33 - protein, 107 aa, synthetic construct
  source 1..107: note = 108VL10.Mb
DIQMTQSPSSLSASVGDRVTITCRASENVGTYISWYQQKPGKAPKLLIYAASNRYTGVPSRFSGSRSGTDFTLTISSLQPEDFATYYCGESYGHLYTFGQGTKVEIK

SEQ ID NO: 34 - protein, 107 aa, synthetic construct
  source 1..107: note = 108VL10.Mc
DIQMTQSPSSLSASVGDRVTITCRASENVGTYISWYQQKPGKAPKLLIYGASSRYTGVPSRFSGSRSGTDFTLTISSLQPEDFATYYCGESYGHLYTFGQGTKVEIK

SEQ ID NO: 35 - protein, 107 aa, synthetic construct
  source 1..107: note = 108VL10.Md
DIQMTQSPSSLSASVGDRVTITCRASENVGTYISWYQQKPGKAPKLLIYGASNRYSGVPSRFSGSRSGTDFTLTISSLQPEDFATYYCGESYGHLYTFGQGTKVEIK

SEQ ID NO: 36 - protein, 107 aa, synthetic construct
  source 1..107: note = 108VL10.Me
DIQMTQSPSSLSASVGDRVTITCRASQSISSYLNWYQQKPGKAPKLLIYGASSRYSGVPSRFSGSRSGTDFTLTISSLQPEDFATYYCGESYGHLYTFGQGTKVEIK

SEQ ID NO: 37 - protein, 117 aa, synthetic construct
  source 1..117: note = 108VH10.Ma
EVQLVESGGGLVQPGGSLRLSCAASGFTFSHHWIHWVRQAPGKGLEWVAMIDPSDSETRLSQKFKDRFTISADTSKNTAYLQMNSLRAEDTAVYYCSRLGRYYFDYWGQGTLVTVSS

SEQ ID NO: 38 - protein, 117 aa, synthetic construct
  source 1..117: note = 108VH10.Mb
EVQLVESGGGLVQPGGSLRLSCAASGYSFTHHWMHWVRQAPGKGLEWVAMIDPSDSETRLSQKFKDRFTISADTSKNTAYLQMNSLRAEDTAVYYCSRLGRYYFDYWGQGTLVTVSS

SEQ ID NO: 39 - protein, 117 aa, synthetic construct
  source 1..117: note = 108VH10.Mc
EVQLVESGGGLVQPGGSLRLSCAASGYSFTHHWISWVRQAPGKGLEWVAMIDPSDSETRLSQKFKDRFTISADTSKNTAYLQMNSLRAEDTAVYYCSRLGRYYFDYWGQGTLVTVSS

SEQ ID NO: 40 - protein, 117 aa, synthetic construct
  source 1..117: note = 108VH10.Md
EVQLVESGGGLVQPGGSLRLSCAASGYSFTHHWIHWVRQAPGKGLEWVAMIKPSDSETRLSQKFKDRFTISADTSKNTAYLQMNSLRAEDTAVYYCSRLGRYYFDYWGQGTLVTVSS

SEQ ID NO: 41 - protein, 117 aa, synthetic construct
  source 1..117: note = 108VH10.Me
EVQLVESGGGLVQPGGSLRLSCAASGYSFTHHWIHWVRQAPGKGLEWVAMIDQSDSETRLSQKFKDRFTISADTSKNTAYLQMNSLRAEDTAVYYCSRLGRYYFDYWGQGTLVTVSS

SEQ ID NO: 42 - protein, 117 aa, synthetic construct
  source 1..117: note = 108VH10.Mf
EVQLVESGGGLVQPGGSLRLSCAASGYSFTHHWIHWVRQAPGKGLEWVAMIDPSDSETRLVDSVKGRFTISADTSKNTAYLQMNSLRAEDTAVYYCSRLGRYYFDYWGQGTLVTVSS

SEQ ID NO: 43 - protein, 117 aa, synthetic construct
  source 1..117: note = 108VH10.Mg
EVQLVESGGGLVQPGGSLRLSCAASGFTFSHHWIHWVRQAPGKGLEWVAMIDASDSETRLSQKFKDRFTISADTSKNTAYLQMNSLRAEDTAVYYCSRLGRYYFDYWGQGTLVTVSS

SEQ ID NO: 44 - protein, 117 aa, synthetic construct
  source 1..117: note = 108VH10.Mh
EVQLVESGGGLVQPGGSLRLSCAASGFTFSHHWIHWVRQAPGKGLEWVAMIDASDSETRLVQKFKDRFTISADTSKNTAYLQMNSLRAEDTAVYYCSRLGRYYFDYWGQGTLVTVSS

SEQ ID NO: 45 - protein, 117 aa, synthetic construct
  source 1..117: note = 108VH10.Mi
EVQLVESGGGLVQPGGSLRLSCAASGFTFSHHWIHWVRQAPGKGLEWVAMIDASDSETRLSDKFKDRFTISADTSKNTAYLQMNSLRAEDTAVYYCSRLGRYYFDYWGQGTLVTVSS

SEQ ID NO: 46 - protein, 117 aa, synthetic construct
  source 1..117: note = 108VH10.Mj
EVQLVESGGGLVQPGGSLRLSCAASGFTFSHHWIHWVRQAPGKGLEWVAMIDASDSETRLSQSFKDRFTISADTSKNTAYLQMNSLRAEDTAVYYCSRLGRYYFDYWGQGTLVTVSS

SEQ ID NO: 47 - protein, 117 aa, synthetic construct
  source 1..117: note = 108VH10.Mk
EVQLVESGGGLVQPGGSLRLSCAASGFTFSHHWIHWVRQAPGKGLEWVAMIDASDSETRLSQKVKDRFTISADTSKNTAYLQMNSLRAEDTAVYYCSRLGRYYFDYWGQGTLVTVSS

SEQ ID NO: 48 - protein, 117 aa, synthetic construct
  source 1..117: note = 108VH10.Ml
EVQLVESGGGLVQPGGSLRLSCAASGFTFSHHWIHWVRQAPGKGLEWVAMIDASDSETRLSQKFKGRFTISADTSKNTAYLQMNSLRAEDTAVYYCSRLGRYYFDYWGQGTLVTVSS

SEQ ID NO: 49 - protein, 117 aa, synthetic construct
  source 1..117: note = 108VH10.Mo
EVQLVESGGGLVQPGGSLRLSCAASGFTFSHHWIHWVRQAPGKGLEWVAMIDASDSETRLSDSFKGRFTISADTSKNTAYLQMNSLRAEDTAVYYCSRLGRYYFDYWGQGTLVTVSS

SEQ ID NO: 50 - protein, 117 aa, synthetic construct
  source 1..117: note = 108VH10.Mp
EVQLVESGGGLVQPGGSLRLSCAASGFTFSHHWIHWVRQAPGKGLEWVAMIDASDSETRLVDSFKGRFTISADTSKNTAYLQMNSLRAEDTAVYYCSRLGRYYFDYWGQGTLVTVSS

SEQ ID NO: 51 - protein, 117 aa, synthetic construct
  source 1..117: note = 108VH10.Mq
EVQLVESGGGLVQPGGSLRLSCAASGFTFSHHWIHWVRQAPGKGLEWVAMIDASDSETRLSDSVKGRFTISADTSKNTAYLQMNSLRAEDTAVYYCSRLGRYYFDYWGQGTLVTVSS

SEQ ID NO: 52 - protein, 117 aa, synthetic construct
  source 1..117: note = 108VH10.Mr
EVQLVESGGGLVQPGGSLRLSCAASGFTFSHHWIHWVRQAPGKGLEWVAMIDASDSETRLVDSVKGRFTISADTSKNTAYLQMNSLRAEDTAVYYCSRLGRYYFDYWGQGTLVTVSS

SEQ ID NO: 53 - protein, 10 aa, synthetic construct
  source 1..10: note = HCDR1 108VH/108VH4.M4/108VH5/108VH5.M8/108VH5.M9/10 8VH10/108VH10.Md/108VH10.Me/108VH10.Mf
GYSFTHHWIH

SEQ ID NO: 54 - protein, 10 aa, synthetic construct
  source 1..10: note = HCDR1 108VH5.M5/108VH5.M10/108VH5.M14/108VH5.M15
GYTFTHHWIH

SEQ ID NO: 55 - protein, 10 aa, synthetic construct
  source 1..10: note = HCDR1 108VH5.M6
GYTFTHHYMH

SEQ ID NO: 56 - protein, 10 aa, synthetic construct
  source 1..10: note = HCDR1 108VH5.M11
GYTFTHHYIH

SEQ ID NO: 57 - protein, 10 aa, synthetic construct
  source 1..10: note = HCDR1 108VH5.M12/M16/M17/M18/M20/M21/M22/M23/M24/M25/M26/M27
GYTFTHHWMH

SEQ ID NO: 58 - protein, 10 aa, synthetic construct
  source 1..10: note = HCDR1 108VH10.Ma/Mg/Mh/Mi/Mj/Mk/Ml/Mo/Mp/Mq/Mr
GFTFSHHWIH

SEQ ID NO: 59 - protein, 10 aa, synthetic construct
  source 1..10: note = HCDR1 108VH10.Mb
GYSFTHHWMH

SEQ ID NO: 60 - protein, 10 aa, synthetic construct
  source 1..10: note = HCDR1 108VH10.Mc
GYSFTHHWIS

SEQ ID NO: 61 - protein, 17 aa, synthetic construct
  source 1..17: note = HCDR2 108VH/108VH5/108VH10/108VH10.Ma/Mb/Mc
MIDPSDSETRLSQKFKD

SEQ ID NO: 62 - protein, 17 aa, synthetic construct
  source 1..17: note = HCDR2 108VH4.M4/108VH5.M5/108VH5.M6/108VH10.Mg
MIDASDSETRLSQKFKD

SEQ ID NO: 63 - protein, 17 aa, synthetic construct
  source 1..17: note = HCDR2 108VH5.M8
MIDASDSETRYAQKFKD

SEQ ID NO: 64 - protein, 17 aa, synthetic construct
  source 1..17: note = HCDR2 108VH5.M9
MIDASDSETRLSQKFQG

SEQ ID NO: 65 - protein, 17 aa, synthetic construct
  source 1..17: note = HCDR2 108VH5.M10/M11/M12
MIDASDSETRYAQKFQG

SEQ ID NO: 66 - protein, 17 aa, synthetic construct
  source 1..17: note = HCDR2 108VH5.M14/M16
MIDASDGETRYAQKFQG

SEQ ID NO: 67 - protein, 17 aa, synthetic construct
  source 1..17: note = HCDR2 108VH5.M15
MIDASDSSTRYAQKFQG

SEQ ID NO: 68 - protein, 17 aa, synthetic construct
  source 1..17: note = HCDR2 108VH5.M17
MIDASEGETRYAQKFQG

SEQ ID NO: 69 - protein, 17 aa, synthetic construct
  source 1..17: note = HCDR2 108VH5.M18
MIDASESETRYAQKFQG

SEQ ID NO: 70 - protein, 17 aa, synthetic construct
  source 1..17: note = HCDR2 108VH5.M20
MIDASDAETRYAQKFQG

SEQ ID NO: 71 - protein, 17 aa, synthetic construct
  source 1..17: note = HCDR2 108VH5.M21
MIDASDFETRYAQKFQG

SEQ ID NO: 72 - protein, 17 aa, synthetic construct
  source 1..17: note = HCDR2 108VH5.M22
MIDASDIETRYAQKFQG

SEQ ID NO: 73 - protein, 17 aa, synthetic construct
  source 1..17: note = HCDR2 108VH5.M23
MIDASDKETRYAQKFQG

SEQ ID NO: 74 - protein, 17 aa, synthetic construct
  source 1..17: note = HCDR2 108VH5.M24
MIDASDLETRYAQKFQG

SEQ ID NO: 75 - protein, 17 aa, synthetic construct
  source 1..17: note = HCDR2 108VH5.M25
MIDASDRETRYAQKFQG

SEQ ID NO: 76 - protein, 17 aa, synthetic construct
  source 1..17: note = HCDR2 108VH5.M26
MIDASDVETRYAQKFQG

SEQ ID NO: 77 - protein, 17 aa, synthetic construct
  source 1..17: note = HCDR2 108VH5.M27
MIDASDYETRYAQKFQG

SEQ ID NO: 78 - protein, 17 aa, synthetic construct
  source 1..17: note = HCDR2 108VH10.Md
MIKPSDSETRLSQKFKD

SEQ ID NO: 79 - protein, 17 aa, synthetic construct
  source 1..17: note = HCDR2 108VH10.Me
MIDQSDSETRLSQKFKD

SEQ ID NO: 80 - protein, 17 aa, synthetic construct
  source 1..17: note = HCDR2 108VH10.Mf
MIDPSDSETRLVDSVKG

SEQ ID NO: 81 - protein, 17 aa, synthetic construct
  source 1..17: note = HCDR2 108VH10.Mh
MIDASDSETRLVQKFKD

SEQ ID NO: 82 - protein, 17 aa, synthetic construct
  source 1..17: note = HCDR2 108VH10.Mi
MIDASDSETRLSDKFKD

SEQ ID NO: 83 - protein, 17 aa, synthetic construct
  source 1..17: note = HCDR2 108VH10.Mj
MIDASDSETRLSQSFKD

SEQ ID NO: 84 - protein, 17 aa, synthetic construct
  source 1..17: note = HCDR2 108VH10.Mk
MIDASDSETRLSQKVKD

SEQ ID NO: 85 - protein, 17 aa, synthetic construct
  source 1..17: note = HCDR2 108VH10.Ml
MIDASDSETRLSQKFKG

SEQ ID NO: 86 - protein, 17 aa, synthetic construct
  source 1..17: note = HCDR2 108VH10.Mo
MIDASDSETRLSDSFKG

SEQ ID NO: 87 - protein, 17 aa, synthetic construct
  source 1..17: note = HCDR2 108VH10.Mp
MIDASDSETRLVDSFKG

SEQ ID NO: 88 - protein, 17 aa, synthetic construct
  source 1..17: note = HCDR2 108VH10.Mq
MIDASDSETRLSDSVKG

SEQ ID NO: 89 - protein, 17 aa, synthetic construct
  source 1..17: note = HCDR2 108VH10.Mr
MIDASDSETRLVDSVKG

SEQ ID NO: 90 - protein, 8 aa, synthetic construct
  source 1..8: note = HCDR3 108VH/all variants
LGRYYFDY

SEQ ID NO: 91 - protein, 11 aa, synthetic construct
  source 1..11: note = LCDR1 108VL
KASENVGTYIS

SEQ ID NO: 92 - protein, 11 aa, synthetic construct
  source 1..11: note = LCDR1 108VL1.M1
RASENVGTYIS

SEQ ID NO: 93 - protein, 11 aa, synthetic construct
  source 1..11: note = LCDR1 108VL1.M5
RASQSVSSYLA

SEQ ID NO: 94 - protein, 11 aa, synthetic construct
  source 1..11: note = LCDR1 108VL10.M1
RASENVGTYIS

SEQ ID NO: 95 - protein, 11 aa, synthetic construct
  source 1..11: note = LCDR1 108VL10.Me
RASQSISSYLN

SEQ ID NO: 96 - protein, 7 aa, synthetic construct
  source 1..7: note = LCDR2 108VL1/108VL1.M1
GASNRYT

SEQ ID NO: 97 - protein, 7 aa, synthetic construct
  source 1..7: note = LCDR2 108VL1.M5
GASNRAT

SEQ ID NO: 98 - protein, 7 aa, synthetic construct
  source 1..7: note = LCDR2 108VL10.Me
GASSRYS

SEQ ID NO: 99 - protein, 9 aa, synthetic construct
  source 1..9: note = LCDR3 108VL/all variants
GESYGHLYT

SEQ ID NO: 100 - protein, 25 aa, synthetic construct
  source 1..25: note = HFR1 VH5-series variants
EVQLVQSGAEVKKPGASVKVSCKAS

SEQ ID NO: 101 - protein, 14 aa, synthetic construct
  source 1..14: note = HFR2 VH5-series variants
WVRQAPGQGLEWMG

SEQ ID NO: 102 - protein, 32 aa, synthetic construct
  source 1..32: note = HFR3 VH5-series variants
RVTMTRDTSTSTVYMELSSLRSEDTAVYYCAR

SEQ ID NO: 103 - protein, 11 aa, synthetic construct
  source 1..11: note = HFR4 VH5-series variants
WGQGTLVTVSS

SEQ ID NO: 104 - protein, 25 aa, synthetic construct [sequence omitted]

SEQ ID NO: 105 - protein, 14 aa, synthetic construct
  source 1..14: note = HFR2 VH10-series variants
WVRQAPGKGLEWVA

SEQ ID NO: 106 - protein, 32 aa, synthetic construct
  source 1..32: note = HFR3 VH10-series variants
RFTISADTSKNTAYLQMNSLRAEDTAVYYCSR

SEQ ID NO: 107 - protein, 11 aa, synthetic construct
  source 1..11: note = HFR4 VH10-series variants
WGQGTLVTVSS

SEQ ID NO: 108 - protein, 23 aa, synthetic construct
  source 1..23: note = LFR1 108VL1.M5
EIVLTQSPATLSLSPGERATLSC

SEQ ID NO: 109 - protein, 15 aa, synthetic construct
  source 1..15: note = LFR2 108VL1.M5
WYQQKPGQAPRLLIY

SEQ ID NO: 110 - protein, 32 aa, synthetic construct
  source 1..32: note = LFR3 108VL1.M5
GIPARFSGSGSGTDFTLTISSLEPEDFAVYYC

SEQ ID NO: 111 - protein, 10 aa, synthetic construct
  source 1..10: note = LFR4 108VL1.M5
FGGGTKVEIK

SEQ ID NO: 112 - protein, 23 aa, synthetic construct
  source 1..23: note = LFR1 108VL10.Me
DIQMTQSPSSLSASVGDRVTITC

SEQ ID NO: 113 - protein, 15 aa, synthetic construct
  source 1..15: note = LFR2 108VL10.Me
WYQQKPGKAPKLLIY

SEQ ID NO: 114 - protein, 32 aa, synthetic construct
  source 1..32: note = LFR3 108VL10.Me
GVPSRFSGSRSGTDFTLTISSLQPEDFATYYC

SEQ ID NO: 115 - protein, 10 aa, synthetic construct
  source 1..10: note = LFR4 108VL10.Me
FGQGTKVEIK

SEQ ID NO: 116 - protein, 10 aa, synthetic construct
  source 1..10: note = HCDR1 Formula
  VARIANT 3: note = X = T or S
  VARIANT 9: note = X = M or I
GYXFTHHWXH

SEQ ID NO: 117 - protein, 17 aa, synthetic construct
  source 1..17: note = HCDR2 Formula
  VARIANT 7: note = X = K, I, or Y
  VARIANT 12: note = X = A or S
  VARIANT 16: note = X = Q or K
MIDASDXETRYXQKFXG

SEQ ID NO: 118 - protein, 10 aa, synthetic construct
  source 1..10: note = HCDR1 Formula
  VARIANT 2: note = X = F or Y
  VARIANT 3: note = X = T or S
  VARIANT 5: note = X = S or T
GXXFXHHWIH

SEQ ID NO: 119 - protein, 17 aa, synthetic construct
  source 1..17: note = HCDR2 Formula
  VARIANT 12: note = X= S or V
  VARIANT 13: note = X = D or Q
  VARIANT 14: note = X = K or S
  VARIANT 15: note = X = F or V
  VARIANT 17: note = X = D or G
MIDASDSETRLXXXXKX

(19) United States
(12) Patent Application Publication
(10) Pub. No.: US 2026/0132197 A1
(43) Pub. Date: May 14, 2026

(54) ANTIBODIES TARGETING CD47 AND USES THEREOF

(71) Applicant: Nanjing Legend Biotech Co., Ltd., Nanjing (CN)

(72) Inventors: Shuai YANG, Nanjing (CN); Xiaojie TU, Nanjing (CN); Yanmin CHEN, Nanjing (CN); Junhua WANG, Nanjing (CN); Yunxiang WU, Nanjing (CN); Xiaoxiao LIU, Nanjing (CN); Shu WU, Nanjing (CN)

(21) Appl. No.: 19/103,422

(22) PCT Filed: Oct. 13, 2023
(86) PCT No.: PCT/CN2023/124387

§ 371 Date: Feb. 12, 2025

(30) Foreign Application Priority Data
Oct. 14, 2022 (WO) PCT/CN2022/125317

(51) Int. Cl. C07K 16/28; A61K 39/00; A61K 47/68; A61K 51/10; A61P 35/00

(52) U.S. Cl. CPC C07K 16/2803; A61K 47/6803; A61K 47/6849; A61K 51/1027; A61K 51/1096; A61P 35/00; A61K 2039/505; A61K 2121/00; C07K 2317/24; C07K 2317/35; C07K 2317/73; C07K 2317/92

(57) ABSTRACT

Provided are an antibody or an antigen-binding fragment thereof that specifically recognizes CD47 and methods of making the same and using the same. The antibodies provided herein exhibit both induction of phagocytosis of cancer cells and have very low to no hemagglutination and phagocytosis of RBCs, showing improved efficacy to toxicity ratio for cancer treatment.

### CROSS REFERENCE TO RELATED APPLICATION

This application is a 371 national stage application of PCT Application No. PCT/CN2023/124387, filed on Oct. 13, 2023, which claims priority to Chinese Application No. PCT/CN2022/125317, filed on Oct. 14, 2022. The disclosures of the aforementioned applications are hereby incorporated by reference in their entireties.

### SEQUENCE STATEMENT

This application contains a Sequence Listing that has been submitted electronically as an XML file named “51624-0111US1_SL_ST26.XML.” The XML file, created on Jan. 7, 2026, is 136,509 bytes in size. The material in the XML file is hereby incorporated by reference in its entirety.

### TECHNICAL FIELD

The present disclosure relates to an antibody or an antigen-binding fragment thereof that specifically recognizes CD47, and methods of making the same and using the same.

### BACKGROUND ART

CD47 (Cluster of Differentiation 47), also known as integrin associated protein (IAP), is a 50-kDa membrane protein with an amino-terminal extracellular IgV domain, a 5-span transmembrane region and a short cytoplasmic tail that is alternatively spliced (Brown E J, Frazier W A 2001; Brown E J 2001). It interacts with multiple ligands, including, without limitation, single-regulatory protein α (SIRPα), SIRPγ, integrins and thrombospondin-1 (TSP-1) (Isenberg J S et. al. 2009).

SIRPα is expressed primarily on myeloid cells, including macrophages, myeloid dendritic cells (DCs), granulocytes, mast cells, and their precursors, including hematopoietic stem cells. CD47/SIRPα interaction transmits a “don't eat me” signal, preventing autologous phagocytosis.

Analysis of patient tumor and matched adjacent normal (non-tumor) tissue revealed that CD47 protein is overexpressed on cancer cells, which efficiently helps them to suppress phagocytic innate immune surveillance and elimination. Blocking the CD47-SIRPα interaction with anti-CD47 antibodies has been shown effective in inducing the phagocytosis of tumor cells in vitro and inhibiting the growth of the various hematological and solid tumors in vivo. Therefore, CD47 is a validated target for cancer therapies and appropriate antagonists of it are needed to make human therapeutics.

Some validated CD47-blocking antibody under clinical investigation induces hemagglutination and anemia (Advani R et al. 2018). In addition, due to ubiquitous CD47 expression on senescent red blood cells (RBCs), the therapeutic utility of CD47-SIRPα blockade monoclonal antibodies is largely compromised due to significant RBC toxicities and fast target-mediated clearance as a result of extensive expression of CD47 on normal cells. A priming strategy has been developed to avoid the RBC depletion toxicities. Patients were first treated at a low dose to remove the aging RBCs, thereby inducing compensatory hematopoiesis (Advani R et. al. 2018). To overcome these limitations, new generation of CD47 antibodies that efficiently target tumor cells while exerting a minimal adverse effect on RBCs to avoid severe anemia were needed.

### SUMMARY OF DISCLOSURE

The present disclosure provides improved antibodies specific for CD47 (e.g., human CD47). The antibodies provided herein exhibit both induction of phagocytosis of cancer cells and have very low to no hemagglutination and phagocytosis of RBCs, showing improved efficacy to toxicity ratio for cancers or proliferative disorders. Also provided are pharmaceutical compositions comprising these antibodies, nucleic acids encoding these antibodies, vectors and host cells for making these antibodies, and methods of treating a subject using these antibodies.

In one aspect, the disclosure provides an antibody or antigen-binding fragment thereof, that binds to CD47, comprising: HCDR1, HCDR2 and HCDR3 of the VH as set forth in SEQ ID NO: 23, 22, 27, 47, 49, 8, 9, 10, 11, 12, 13, 14, 15, 16, 17, 18, 19, 20, 21, 24, 25, 26, 37, 38, 39, 40, 41, 42, 43, 44, 45, 46, 48, 50, 51, or 52; and LCDR1, LCDR2 and LCDR3 of the VL as set forth in SEQ ID NO: 7 or 36.

In certain embodiments, the antibody or antigen-binding fragment thereof of the disclosure comprises: HCDR1, HCDR2 and HCDR3 of the VH as set forth in SEQ ID NO: 23, 22, 27, 8, 9, 10, 11, 12, 13, 14, 15, 16, 17, 18, 19, 20, 21, 24, 25, or 26; and LCDR1, LCDR2 and LCDR3 of the VL as set forth in SEQ ID NO: 7; preferably, the HCDR1 is defined according to AbM numbering system and HCDR2, HCDR3, LCDR1, LCDR2 and LCDR3 are defined according to Kabat numbering system.

In certain embodiments, the antibody or antigen-binding fragment thereof of the disclosure comprising:
(i) HCDR1 comprising the sequence of GYX₁FTHHWX₂H (SEQ ID NO: 116), wherein X₁ is T or S, X₂ is M or I;
(ii) HCDR2 comprising the sequence of MIDASDX₃ETRYX₄QKFX₅G (SEQ ID NO: 117), wherein X₃ is K, I, or Y, X₄ is A or S, X₅ is Q or K;
(iii) HCDR3 comprising the sequence of SEQ ID NO: 90;
(iv) LCDR1 comprising the sequence of SEQ ID NO: 93;
(v) LCDR2 comprising the sequence of SEQ ID NO: 97; and,
(vi) LCDR3 comprising the sequence of SEQ ID NO: 99.

In certain embodiments, X₁ is T, and/or, X₂ is M; preferably, the HCDR1 comprises the sequence of SEQ ID NO: 57.

In certain embodiments, X₄ is A, and/or, X₅ is Q; preferably, the HCDR2 comprises the sequence of SEQ ID NO: 73, 72, or 77.

In certain embodiments, the antibody or antigen-binding fragment thereof of the disclosure comprising:
HCDR1, HCDR2 and HCDR3 comprising the amino acid sequences of SEQ ID NOs: 57, 73, and 90, respectively; and LCDR1, LCDR2 and LCDR3 comprising the amino acid sequences of SEQ ID NOs: 93, 97, and 99, respectively;
HCDR1, HCDR2 and HCDR3 comprising the amino acid sequences of SEQ ID NOs: 57, 72, and 90, respectively; and LCDR1, LCDR2 and LCDR3 comprising the amino acid sequences of SEQ ID NOs: 93, 97, and 99, respectively; or,
HCDR1, HCDR2 and HCDR3 comprising the amino acid sequences of SEQ ID NOs: 57, 77, and 90, respectively; and LCDR1, LCDR2 and LCDR3 comprising the amino acid sequences of SEQ ID NOs: 93, 97, and 99, respectively.

In certain embodiments, the antibody or antigen-binding fragment thereof of the disclosure comprising:
HCDR1 comprising the amino acid sequence of SEQ ID NO: 57, HCDR2 comprising the amino acid sequence of SEQ ID NO: 65, 66, 68, 69, 70, 71, 74, 75, or 76, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 56, HCDR2 comprising the amino acid sequence of SEQ ID NO: 65, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 55, HCDR2 comprising the amino acid sequence of SEQ ID NO: 62, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 54, HCDR2 comprising the amino acid sequence of SEQ ID NO: 62, 65, 66, or 67, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 53, HCDR2 comprising the amino acid sequence of SEQ ID NO: 63 or 64, HCDR3 comprising the amino acid sequence of SEQ ID NO: 90; and LCDR1, LCDR2 and LCDR3 comprising the amino acid sequences of SEQ ID NOs: 93, 97, and 99, respectively.

In certain embodiments, the antibody or antigen-binding fragment thereof of the disclosure further comprising: four heavy chain framework regions (HFR1, HFR2, HFR3, and HFR4) comprising the amino acid sequences of SEQ ID NOs: 100, 101, 102 and 103, respectively; and/or, four light chain framework regions (LFR1, LFR2, LFR3, and LFR4) comprising the amino acid sequences of SEQ ID NOs: 108, 109, 110 and 111, respectively.

In certain embodiments, the antibody or antigen-binding fragment thereof of the disclosure comprising: a heavy chain variable region (VH) and a light chain variable region (VL), wherein:
the VH comprises the sequence of SEQ ID NO: 23 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 22 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 27 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NOs: 8, 9, 10, 11, 12, 13, 14, 15, 16, 17, 18, 19, 20, 21, 24, 25, or 26 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: HCDR1, HCDR2 and HCDR3 of the VH as set forth in SEQ ID NO:47, 49, 50, 51, 52, 37, 38, 39, 40, 41, 42, 43, 44, 45, 46, or 48; and LCDR1, LCDR2 and LCDR3 of the VL as set forth in SEQ ID NO: 36; preferably, the HCDR1 is defined according to AbM numbering system and HCDR2, HCDR3, LCDR1, LCDR2 and LCDR3 are defined according to Kabat numbering system.

In certain embodiments, the antibody or antigen-binding fragment thereof of the disclosure comprising:
(i) HCDR1 comprising the sequence of GX₁X₂FX₃HHWIH (SEQ ID NO: 118), wherein X₁ is F or Y, X₂ is T or S, X₃ is S or T;
(ii) HCDR2 comprising the sequence of MIDASDSETRLX₄X₅X₆X₇KX₈ (SEQ ID NO: 119), wherein X₄ is S or V, X₅ is D or Q, X₆ is K or S, X₇ is F or V, X₈ is D or G;
(iii) HCDR3 comprising the sequence of SEQ ID NO: 90;
(iv) LCDR1 comprising the sequence of SEQ ID NO: 95;
(v) LCDR2 comprising the sequence of SEQ ID NO: 98; and,
(vi) LCDR3 comprising the sequence of SEQ ID NO: 99.

In certain embodiments, X₁ is F, X₂ is T, and/or, X₃ is S; preferably, the HCDR1 comprises the sequence of SEQ ID NO: 58.

In certain embodiments, X₄ is S, X₅ is D or Q, X₆ is K or S, X₇ is F or V, and X₈ is D or G.

In certain embodiments, X₄ is S or V, X₅ is D, X₆ is S, X₇ is F or V, and X₈ is G; preferably, X₄ is S and/or X₇ is F.

In certain embodiments, the HCDR2 comprises the sequence of SEQ ID NO: 84, 86, 87, 88, or 89.

In certain embodiments, the antibody or antigen-binding fragment thereof of the disclosure comprising:
HCDR1, HCDR2 and HCDR3 comprising the amino acid sequences of SEQ ID NOs: 58, 84, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 86, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 87, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 88, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 89, and 90, respectively; and LCDR1, LCDR2 and LCDR3 comprising the amino acid sequences of SEQ ID NOs: 95, 98, and 99, respectively.

In certain embodiments, the antibody or antigen-binding fragment thereof of the disclosure comprising:
HCDR1 comprising the amino acid sequence of SEQ ID NO: 58; HCDR2 comprising the amino acid sequence of SEQ ID NO: 61, 62, 81, 82, 83, or 85; HCDR3 comprising the amino acid sequence of SEQ ID NO: 90; and LCDR1, LCDR2 and LCDR3 comprising the amino acid sequences of SEQ ID NOs: 95, 98, and 99, respectively; or
HCDR1 comprising the amino acid sequence of SEQ ID NO: 53; HCDR2 comprising the amino acid sequence of SEQ ID NO: 80; HCDR3 comprising the amino acid sequence of SEQ ID NO: 90; and LCDR1, LCDR2 and LCDR3 comprising the amino acid sequences of SEQ ID NOs: 95, 98, and 99, respectively; or
HCDR1 comprising the amino acid sequence of SEQ ID NO: 59 or 60; HCDR2 comprising the amino acid sequence of SEQ ID NO: 61; HCDR3 comprising the amino acid sequence of SEQ ID NO: 90; and LCDR1, LCDR2 and LCDR3 comprising the amino acid sequences of SEQ ID NOs: 95, 98, and 99, respectively; or
HCDR1 comprising the amino acid sequence of SEQ ID NO: 53; HCDR2 comprising the amino acid sequence of SEQ ID NO: 78 or 79; HCDR3 comprising the amino acid sequence of SEQ ID NO: 90; and LCDR1, LCDR2 and LCDR3 comprising the amino acid sequences of SEQ ID NOs: 95, 98, and 99, respectively.

In certain embodiments, the antibody or antigen-binding fragment thereof of the disclosure further comprising: four heavy chain framework regions (HFR1, HFR2, HFR3, HFR4) comprising the amino acid sequences of SEQ ID NOs: 104, 105, 106 and 107, respectively; and/or, four light chain framework regions (LFR1, LFR2, LFR3, LFR4) comprising the amino acid sequences of SEQ ID NOs: 112, 113, 114 and 115, respectively.

In certain embodiments, the antibody or antigen-binding fragment thereof of the disclosure comprising a heavy chain variable region (VH) and a light chain variable region (VL), wherein:
the VH comprises the sequence of SEQ ID NO: 47 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 49 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 50 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 51 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 52 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises the sequence of SEQ ID NO: 37, 38, 39, 40, 41, 42, 43, 44, 45, 46, or 48 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure as defined in any one of the preceding embodiments can further comprise a heavy chain constant region (CH) comprising an amino acid sequence derived from a human immunoglobulin heavy chain constant region.

In certain embodiments, the heavy chain constant region is an IgG heavy chain constant region, such as an IgG1, IgG2, IgG3 or IgG4 heavy chain constant region. In certain embodiments, the heavy chain constant region is an IgG4 heavy chain constant region, e.g., with S228P substitution. In certain embodiments, the heavy chain constant region comprises the amino acid sequence of SEQ ID NO: 28.

In certain embodiments, the heavy chain constant region has effector function (e.g., ADCC and/or ADCP) or enhanced effector function (e.g., ADCC and/or ADCP).

In certain embodiments, the antibody or antigen-binding fragment thereof of the disclosure as defined in any one of the preceding embodiments can further comprise a light chain constant region (CL) comprising an amino acid sequence derived from a human immunoglobulin light chain constant region.

In certain embodiments, the light chain constant region is a kappa light chain constant region. In certain embodiments, the light chain constant region comprises the amino acid sequence of SEQ ID NO: 29.

In certain embodiments, the antibody or antigen-binding fragment thereof of the disclosure comprising a heavy chain and a light chain, wherein:
the heavy chain comprises a VH comprising the sequence of SEQ ID NO: 23 and a CH comprising the sequence of SEQ ID NO: 28; and the light chain comprises a VL comprising the sequence of SEQ ID NO: 7 and a CL comprising the sequence of SEQ ID NO: 29; or,
the heavy chain comprises a VH comprising the sequence of SEQ ID NO: 22 and a CH comprising the sequence of SEQ ID NO: 28; and the light chain comprises a VL comprising the sequence of SEQ ID NO: 7 and a CL comprising the sequence of SEQ ID NO: 29; or,
the heavy chain comprises a VH comprising the sequence of SEQ ID NO: 27 and a CH comprising the sequence of SEQ ID NO: 28; and the light chain comprises a VL comprising the sequence of SEQ ID NO: 7 and a CL comprising the sequence of SEQ ID NO: 29; or,
the heavy chain comprises a VH comprising the sequence of SEQ ID NO: 47 and a CH comprising the sequence of SEQ ID NO: 28; and the light chain comprises a VL comprising the sequence of SEQ ID NO: 36 and a CL comprising the sequence of SEQ ID NO: 29; or,
the heavy chain comprises a VH comprising the sequence of SEQ ID NO: 49 and a CH comprising the sequence of SEQ ID NO: 28; and the light chain comprises a VL comprising the sequence of SEQ ID NO: 36 and a CL comprising the sequence of SEQ ID NO: 29.

In certain embodiments, the antibody or antigen-binding fragment thereof of the disclosure as defined in any one of the preceding embodiments is selected from the group consisting of scFv, Fab, Fab′, (Fab′)₂, Fv fragments, diabodies, bispecific antibodies, multispecific antibodies, and humanized antibodies.

In certain embodiments, the antibody or antigen-binding fragment thereof of the disclosure binds to wild-type human CD47 with a K_{D} of about 10⁻⁶ M to about 10⁻¹⁰ M, e.g., about 10⁻⁷ M to about 10⁻⁹ M, about 10⁻⁶ M to about 10⁻⁸ M, or about 10⁻⁷ M to about 10⁻⁸ M, or about 10⁻⁸ M to about 10⁻⁹ M.

In certain embodiments, the antibody or antigen-binding fragment thereof of the disclosure binds to human CD47 mutein with C33G mutation (e.g., shown as SEQ ID NO: 1) with a K_{D} of about 10⁻⁶ M to about 10⁻¹⁰ M, e.g., about 10⁻⁷ M to about 10⁻⁹ M, about 10⁻⁶ M to about 10⁻⁸ M, or about 10⁻⁷ M to about 10⁻⁸ M, or about 10⁻⁸ M to about 10⁻⁹ M.

In certain embodiments, the antibody or antigen-binding fragment thereof of the disclosure inhibits, blocks, antagonizes, neutralizes or otherwise interferes with CD47 expression, activity and/or signaling, induces the phagocytosis of tumor cells, and inhibits the growth of the various hematological and solid tumors.

In certain embodiments, the antibody or antigen-binding fragment thereof of the disclosure induces no obvious hemagglutination and phagocytosis of RBCs.

In certain embodiments, the antibody or antigen-binding fragment thereof of the disclosure has improved efficacy to toxicity ratio.

In another aspect, the disclosure provides an immunoconjugate, comprising the antibody or an antigen binding fragment thereof of the disclosure and an effector molecule. In certain embodiments, the effector molecule is a therapeutic agent. In certain embodiments, the therapeutic agent is selected from the group consisting of a drug, a toxin, a radioisotope, a protein, a peptide, and a nucleic acid.

In another aspect, the disclosure provides a bispecific or multispecific antibody, comprising the antibody or an antigen binding fragment thereof of the disclosure. In certain embodiments, the bispecific or multispecific antibody specifically binds to CD47 (e.g., human CD47) and a second target. In certain embodiments, the bispecific or multispecific antibody comprises a first antigen binding domain from the antibody or an antigen binding fragment thereof of the disclosure and a second antigen binding domain from an antibody against a second target. In certain embodiments, the second target is an immunomodulatory receptor or tumor-associated antigen.

In another aspect, the disclosure provides an isolated nucleic acid molecule, comprising a nucleotide sequence encoding the antibody or an antigen binding fragment thereof of the disclosure, or the heavy chain variable region and/or light chain variable region thereof, or the bispecific or multispecific antibody of the disclosure.

In another aspect, the disclosure provides a vector (e.g., a cloning vector or an expression vector), comprising the isolated nucleic acid molecule of the disclosure.

In another aspect, the disclosure provides a host cell, comprising the isolated nucleic acid molecule of the disclosure or the vector of the disclosure.

In another aspect, provided is a method for producing the antibody or antigen-binding fragment thereof of the disclosure, or the bispecific or multispecific antibody of the disclosure, comprising, culturing a host cell comprising the isolated nucleic acid molecule of the disclosure or the vector of the disclosure or the host cell of the disclosure under a condition allowing expression of the antibody or antigen-binding fragment thereof or the bispecific or multispecific antibody, and recovering the antibody or antigen-binding fragment thereof or the bispecific or multispecific antibody from a culture of the cultured host cell.

In another aspect, the disclosure provides a pharmaceutical composition, comprising the antibody or antigen-binding fragment thereof of the disclosure, or the immunoconjugate of the disclosure, or the bispecific or multispecific antibody of the disclosure, or the isolated nucleic acid molecule, vector, or host cell of the disclosure, and a pharmaceutically acceptable carrier and/or excipient. In certain embodiments, the pharmaceutical composition of the disclosure may further comprise an additional therapeutic agent.

In certain embodiments, the additional therapeutic agent is an anti-tumor agent.

In certain embodiments, the additional therapeutic agent is an additional therapeutic antibody for cancer treatment, e.g, a therapeutic antibody with ADCC and/or ADCP activity or enhanced ADCC and/or ADCP activity. In certain embodiments, the additional therapeutic antibody binds to a target other than CD47. In certain embodiments, the target is selected from an immunomodulatory receptor or tumor-associated antigen.

In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide.

In another aspect, provided is use of the antibody or an antigen-binding fragment thereof of the disclosure, or the immunoconjugate of the disclosure, or the bispecific or multispecific antibody of the disclosure, or the isolated nucleic acid molecule, vector, or host cell of the disclosure, or the pharmaceutical composition of the disclosure, in the manufacture of a medicament for use in treating a cancer or proliferative disorder in a subject.

In another aspect, provided is the antibody or an antigen-binding fragment thereof of the disclosure, or the immunoconjugate of the disclosure, or the bispecific or multispecific antibody of the disclosure, or the isolated nucleic acid molecule, vector, or host cell of the disclosure, or the pharmaceutical composition of the disclosure, for use in treating a cancer or proliferative disorder in a subject.

In another aspect, provided is a method of treating a cancer or proliferative disorder in a subject; wherein the method comprising administering to a subject in need thereof an effective amount of the antibody or an antigen-binding fragment thereof of the disclosure, or the immunoconjugate of the disclosure, or the bispecific or multispecific antibody of the disclosure, or the isolated nucleic acid molecule, vector, or host cell of the disclosure, or the pharmaceutical composition of the disclosure.

In certain embodiments, in any of the methods or uses of treatment described herein, the antibody or antigen-binding fragment thereof of the disclosure inhibits, blocks, antagonizes, neutralizes or otherwise interferes with CD47 expression, activity and/or signaling, induces the phagocytosis of tumor cells, and inhibits the growth of the various hematological and solid tumors.

In certain embodiments, in any of the methods or uses of treatment described herein, the antibody or antigen-binding fragment thereof of the disclosure induces no obvious hemagglutination and phagocytosis of RBCs.

In certain embodiments, in any of the methods or uses of treatment described herein, the antibody or antigen-binding fragment thereof of the disclosure has improved efficacy to toxicity ratio.

In certain embodiments, in any of the methods or uses of treatment described herein, the cancer or proliferative disorder is associated with CD47 and/or SIRPα expression.

In certain embodiments, in any of the methods or uses of treatment described herein, the cancer or proliferative disorder is a hematological cancer, e.g., leukemia, lymphoma or myeloma. In certain embodiments, the hematological cancer is a leukemia selected from the group consisting of acute lymphocytic leukemia (ALL), acute myeloid leukemia (AML), chronic lymphocytic leukemia (CLL), chronic myelogenous leukemia (CML), Myeloproliferative disorder/neoplasm (MPDS), and myelodysplastic syndrome (MDS). In certain embodiments, the hematological cancer is a lymphoma selected from the group consisting of a Hodgkin's lymphoma, both indolent and aggressive non-Hodgkin's lymphoma, Burkitt's lymphoma, and follicular lymphoma (small cell and large cell). In certain embodiments, the hematological cancer is a myeloma selected from the group consisting of multiple myeloma (MM), giant cell myeloma, heavy-chain myeloma, and light chain or Bence-Jones myeloma.

In certain embodiments, in any of the methods or uses of treatment described herein, the cancer or proliferative disorder is a solid tumor, e.g., breast cancer, ovarian cancer, head and neck cancer, bladder cancer, melanoma, colorectal cancer, pancreatic cancer, lung cancer, leiomyoma, leiomyosarcoma, glioma, glioblastoma, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

In certain embodiments, in any of the methods or uses of treatment described herein, the subject is a mammal, such as a human.

In certain embodiments, in any of the methods or uses of treatment described herein, the antibody or antigen-binding fragment thereof, or the immunoconjugate of the disclosure, or the bispecific or multispecific antibody of the disclosure, or the pharmaceutical composition is used in combination with an additional therapeutic agent or an additional therapy.

In certain embodiments, the additional therapeutic agent is an anti-tumor agent.

In certain embodiments, the additional therapeutic agent is an additional therapeutic antibody for cancer treatment, e.g, a therapeutic antibody with ADCC and/or ADCP activity or enhanced ADCC and/or ADCP activity. In certain embodiments, the additional therapeutic antibody binds to a target other than CD47. In certain embodiments, the target is selected from an immunomodulatory receptor or tumor-associated antigen.

In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide.

In certain embodiments, the additional therapy is a standard cancer treatment, such as surgery, chemotherapy, radiation therapy, targeted therapy, immunotherapy, hormone therapy, gene therapy or palliative care.

Further features and advantages of certain embodiments of the present disclosure will become more fully apparent in the following description of the embodiments and drawings thereof, and from the claims.

### BRIEF DESCRIPTION OF THE DRAWINGS

FIG. 1. Binding of anti-CD47 antibodies M6 (FIG. 1a), M11 (FIG. 1b), M15 (FIG. 1c), M16 (FIG. 1d), M21 (FIG. 1e), M22 (FIG. 1f), M23 (FIG. 1g), M24 (FIG. 1h), M25 (FIG. 1i), M27 (FIG. 1j), Mk (FIG. 1k), Mo (FIG. 1l), Mp (FIG. 1m), Mq (FIG. 1n) and Mr (FIG. 1o) to SHP-77 tumor line. M4, Hu5F9, TJC4 antibodies, TTI-622 and isotype control antibody were used in every figures for comparison.

FIG. 2. Binding of anti-CD47 antibodies M6 (FIG. 2a), M11 (FIG. 2b), M15 (FIG. 2c), M16 (FIG. 2d), M21 (FIG. 2e), M22 (FIG. 2f), M23 (FIG. 2g), M24 (FIG. 2h), M25 (FIG. 2i), M27 (FIG. 2j), Mk (FIG. 2k), Mo (FIG. 2l), Mp (FIG. 2m), Mq (FIG. 2n) and Mr (FIG. 2o) to human red blood cells. M4, Hu5F9, TJC4 antibodies, TTI-622 and isotype control antibody were used in every figures for comparison.

FIG. 3. Binding of anti-CD47 antibodies M4 (FIG. 3a), M6 (FIG. 3b), M16 (FIG. 3c), M22 (FIG. 3d), M23 (FIG. 3e), M27 (FIG. 3f), Mk (FIG. 3g), Mq (FIG. 3h), Mr (FIG. 3i), Hu5F9 (FIG. 3j), TJC4 (FIG. 3k) and TTI-622 (FIG. 3l) to human and cynomolgus CD47 CHO-K1 stable cell lines.

FIG. 4. Phagocytosis of CCRF-CEM tumor cells induced by anti-CD47 antibodies M21 (FIG. 4a), M22 (FIG. 4b), M23 (FIG. 4c), M27 (FIG. 4d), Mk (FIG. 4e), Mo (FIG. 4f), Mp (FIG. 4g), Mq (FIG. 4h) and Mr (FIG. 4i). Hu5F9 and TJC4 antibodies, TTI-622 and isotype control antibody were used in every figures for comparison.

FIG. 5. Phagocytosis of human red blood cells by anti-CD47 antibodies. Phagocytosis of RBCs induced by Hu5F9 at 6.4 pM and 32 pM, and by the rest of antibodies at 32 pM and 160 pM was carried out at effector-to-target ratio of 1:1.

FIG. 6. Phagocytosis of CCRF-CEM tumor cells induced by selected variants of humanized 108C10A6 antibody. Hu5F9 and isotype control antibody were used as positive and negative controls, respectively.

FIG. 7. Comparison of the phagocytosis of human red blood cells and tumor cells by selected anti-CD47 antibodies at fixed concentrations (Hu5F9 at 6.4 pM and 32 pM, the other antibodies at 32 pM and 160 pM, and isotype control antibody at 6.4 pM, 32 pM and 160 pM).

FIG. 8. Hemagglutination of human red blood cells induced by anti-CD47 antibodies.

FIG. 9. In vivo efficacy of humanized anti-CD47 antibodies. FIG. 9a shows the change of the average tumor volumes as mice were dosed with anti-CD47 antibodies, benchmarks (TJC4 and TTI-622) and isotype control. FIG. 9b-9m are spider plots showing the change in tumor volume for each tumor-bearing mouse upon treatment. FIG. 9n shows the change of average body weight of tumor-bearing mice upon treatment.

### DETAILED DESCRIPTION

### Definitions

In the present disclosure, the scientific and technical terms used herein have the meanings commonly understood by those skilled in the art, unless otherwise stated. For better understanding of the present disclosure, definitions and explanations of terms are provided below.

As used herein, the term “CD47” refers to Cluster of Differentiation 47, also known as integrin associated protein (IAP). CD47 is a 50-kDa membrane protein with an amino-terminal extracellular IgV domain, a 5-span transmembrane region and a short cytoplasmic tail that is alternatively spliced. An exemplary amino acid sequence of human CD47 protein is provided in NCBI Reference Sequence: NP_942088.1. In the sequence of the IgV domain of CD47, the first cysteine residue, Cys33 was confirmed to form a disulfide bond with Cys263 of the multiply membrane-spanning (MMS) domain. The existence of the free Cys33 in the Ig^{V} domain may induce the formation of CD47 IgV domain homodimer. Therefore, in addition to wild-type CD47, the term “CD47” also contains CD47 mutein with C33G mutation. Such mutein can be used for the determination of true mono-valent binding affinity of anti-CD47 antibody.

As used herein, the term “antibody” refers to an immunoglobulin molecule capable of specific binding to a target (such as a carbohydrate, polynucleotide, lipid, polypeptide, etc.) through at least one antigen recognition site, located in the variable region of the immunoglobulin molecule. Unless otherwise indicated or clear from the context, the term “antibody” as used to herein may include whole antibodies and any antigen binding fragments (i.e., “antigen-binding portions”) or single chains thereof. In one embodiment, “antibody” is typically composed of two pairs of polypeptide chains (each pair has a “light” (L) chain and a “heavy” (H) chain). Antibody light chains can be classified as κ and λ light chains. Heavy chains can be classified as μ, δ, γ, α, or ε, and the isotypes of antibody are defined as IgM, IgD, IgG, IgA, and IgE, respectively. Within the light and heavy chains, variable region and constant region are joined by a “J” region of about 12 or more amino acids, and the heavy chain further comprises a “D” region of about 3 or more amino acids. Each heavy chain consists of a heavy chain variable region (V_{H}) and a heavy chain constant region (C_{H}). The heavy chain constant region consists of three domains (C_{H}1, C_{H}2 and C_{H}3). Each light chain consists of a light chain variable region (V_{L}) and a light chain constant region (C_{L}). The light chain constant region consists of one domain C_{L}. The V_{H} and V_{L} regions can also be subdivided into hypervariable regions (referred to as complementarity determining regions (CDRs)) interspaced with relatively conservative regions called framework regions (FR). Each of V_{H} and V_{L} consists of three CDRs and four FRs arranged in the following order: FR1, CDR1, FR2, CDR2, FR3, CDR3, FR4 from the amino terminus to the carboxy terminus. The variable regions (V_{H} and V_{L}) of each heavy/light chain pair form an antibody binding site, respectively. The term “antibody” is not limited by any particular method for producing the antibody, for example, it comprises recombinant antibodies, monoclonal antibodies, and polyclonal antibodies.

As used herein, the term “complementarity determining region” or “CDR” refers to the amino acid residues in the variable region of an antibody that are responsible for antigen binding. The precise boundaries of these amino acid residues can be defined according to various numbering systems known in the art, for example, according to the definitions in the Kabat numbering system (Kabat et al., Sequences of Proteins of Immunological Interest, 5th Ed. Public Health Service, National Institutes of Health, Bethesda, Md., 1991), Chothia numbering system (Chothia & Lesk (1987) J. Mol. Biol. 196: 901-917; Chothia et al. (1989) Nature 342: 878-883), AbM numbering system (Martin, in Antibody Engineering, Vol. 2, Chapter 3, Springer Verlag) or IMGT numbering system (Lefranc et al., Dev. Comparat. Immunol. 27: 55-77, 2003). For a given antibody, those skilled in the art can easily identify the CDRs defined by each numbering system. Moreover, the correspondence between different numbering systems is well known to those skilled in the art (e.g., see Lefranc et al., Dev. Comparat. Immunol. 27: 55-77, 2003). In certain embodiments, the CDRs of the antibodies of the disclosure are defined according to Kabat, AbM, IMGT, or Chothia numbering system, or any combination thereof. Unless otherwise indicated or clear from the context, the HCDR1 of the antibodies of the disclosure are preferably defined according to AbM numbering system and HCDR2, HCDR3, LCDR1, LCDR2 and LCDR3 are defined according to Kabat numbering system.

As used herein, the term “framework region” or “FR” residues refers to those amino acid residues other than the CDR residues as defined above in the variable regions of antibody.

As used herein, the term “antigen-binding fragment” of an antibody refers to a polypeptide comprising a fragment of a full-length antibody, which retains the ability to specifically bind to the same antigen to which the full-length antibody binds, and/or competes with the full-length antibody to specifically bind to the antigen, and which is also referred to as an “antigen-binding portion”. An antigen-binding fragment of an antibody can be produced by recombinant DNA techniques or by enzymatic or chemical cleavage of the intact antibody. In certain embodiments, the antigen-binding fragment comprises Fab, Fab′, F(ab′)₂, Fd, Fv, dAb and fragments of complementarity determining regions (CDRs), single chain antibodies (e.g., scFv), chimeric antibodies, diabodies, and polypeptides comprising at least a portion of the antibody that is sufficient to confer the specific antigen binding ability to the polypeptide.

As used herein, the term “Fd fragment” refers to an antibody fragment consisting of V_{H} and C_{H}1 domains; the term “dAb fragment” refers to an antibody fragment consisting of V_{H} domain; the term “Fab fragment” refers to an antibody fragment consisting of V_{L}, V_{H}, C_{L} and C_{H}1 domains; the term “F(ab′)₂ fragment” refers to an antibody fragment comprising two Fab fragments linked by a disulfide bridge at the hinge region.

As used herein, the term “Fv fragment” refers to an antibody fragment consisting of V_{L} and V_{H} domains of a single arm of an antibody. An Fv fragment is generally considered to be the smallest antibody fragment that can form a complete antigen binding site. It is believed that six CDRs confer the antigen binding specificity to the antibody. However, even one variable region (e.g., an Fd fragment, which contains only three CDRs specific for an antigen) is able to recognize and bind an antigen, albeit with less affinity than that of the entire binding site.

As used herein, the term “scFv” refers to a single polypeptide chain comprising VL and VH domains, having a general structure of NH₂-VL-linker-VH-COOH or NH₂-VH-linker-VL-COOH. A suitable linker of prior art consists of a repeated GGGGS amino acid sequence or variants thereof. For example, a linker having amino acid sequence (GGGGS)₄ can be used, but variants thereof can also be used. In some cases, there may also be a disulfide bond between the VH and VL of scFv.

As used herein, the term “diabody” refers to a dimer of scFv which consists of VH and VL domains connected by a short peptide linker. The linker is too short to form intrachain pairing of VH and VL domains. Instead, two such scFv fragments are co-expressed to form multimers by inter-chain pairing (cross-over pairing) of VH and VL domains.

Each of the antigen-binding fragments maintains the ability to specifically bind to the same antigen to which the full-length antibody binds, and/or compete with the full-length antibody for specific binding to the antigen. An antigen-binding fragment can be obtained from a given antibody (e.g., an intact antibody provided herein) using conventional techniques known to those skilled in the art (e.g., recombinant DNA techniques or enzymatic or chemical cleavage methods), and can be screened for specificity in the same manner by which intact antibodies are screened.

As used herein, the term “humanized antibody” refers to a genetically engineered non-human antibody of which the amino acid sequence has been modified to increase its homology to the sequence of a human antibody. Generally, all or part of the CDR regions of a humanized antibody are derived from a non-human antibody (donor antibody), and all or part of the non-CDR regions (e.g., variable region FR and/or constant region) are derived from a human immunoglobulin (receptor antibody).

To prepare a humanized antibody, murine CDR regions can be grafted onto a human framework sequence by using any methods known in the art. Two strategies were used in this application to select a human acceptor framework, including using the human germline gene that is most closely related to the parent murine antibody or using a well-behaved “fixed framework”. Some CDR residues were either mutated to their human counterparts to increase the humanness of the antibody or to some other residues to alter the binding affinity of the antibody to a certain level that the toxicities to cancer cells was maintained while the toxicities to RBCs were minimized.

As used herein, the term “bispecific antibody” refers to an artificial hybrid antibody having two different heavy/light chain pairs, giving rise to two antigen binding sites with specificity for different antigens. Bispecific antibodies can be produced by a variety of methods, including linking of a first antibody or its fragment and a second antibody or its fragment, for example, by chemical coupling, gene fusion, non-covalent association, or other means. “Multispecific antibody” refers to an artificial hybrid antibody that has more than two different binding specificities, including for example, trispecific antibody or tetraspecific antibody.

As used herein, the term “specifically bind” or “specific for” refers to the binding of two molecules in a non-random manner, such as the reaction between an antibody and the antigen it directs to. In some embodiments, an antibody that specifically binds to an antigen (or an antibody specific for an antigen) refers to an antibody that binds to the antigen with an affinity (K_{D}) of less than about 10⁻⁵ M, e.g., less than about 10⁻⁶ M, 10⁻⁷ M, 108 M, 10⁻⁹ M, or 10⁻¹⁰ M or less.

As used herein, the term “K_{D}” refers to a dissociation constant of a specific antibody-antigen interaction, which is used to describe the binding affinity of an antibody to an antigen. The smaller the dissociation constant, the more tightly bound the antibody is, and the higher the affinity between antibody and antigen. Generally, an antibody (e.g., the antibody of the disclosure) binds to an antigen (e.g., human CD47) with a K_{D} of less than about 10⁻⁵ M, e.g., less than about 10⁻⁶ M, 10⁻⁷ M, 10⁻⁸ M, 10⁻⁹ M, or 10⁻¹⁰ M or less, determined by, for example, surface plasmon resonance (SPR) in BIACORE device. In certain embodiments, the antibodies disclosed herein bind to human CD47 with a K_{D} of about 10⁻⁶ M to about 10⁻¹⁰ M, e.g., about 10⁻⁷ M to about 10⁻⁹ M, about 10⁻⁶ M to about 10⁻⁸ M, or about 10⁻⁷ M to about 10⁻⁸ M, or about 10⁻⁸ M to about 10⁻⁹ M.

As used herein, the term “vector” refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element, and a reporter gene. In addition, a vector may comprise origin of replication.

As used herein, the term “host cell” refers to a cell into which a vector can be introduced or transformed, including, but not limited to, prokaryotic cells such as E. coli or Bacillus subtilis, and eukaryotic cells such as mammal cells (e.g., mouse cell or human cell), insect cells or yeast cells. Suitable eukaryotic cells include, but not limited to, NS0 cells, Vero cells, Hela cells, COS cells, CHO cells, HEK293 cells, BHK cells or MDCKII cells.

As used herein, the term “identity” refers to the match degree between two polypeptides or between two nucleic acids. When two sequences for comparison have the same monomer sub-unit of base or amino acid at a certain site (e.g., each of two DNA molecules has an adenine at a certain site, or each of two polypeptides has a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Generally, the comparison of two sequences is conducted in a manner to produce maximum identity. Such alignment can be conducted by using a computer program such as Align program (DNAstar, Inc.) which is based on the method of Needleman, et al. (J. Mol. Biol. 48:443-453, 1970). The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “pharmaceutically acceptable carrier and/or excipient” refers to a carrier and/or excipient that is pharmacologically and/or physiologically compatible with a subject and an active ingredient, which is well known in the art (see, for example, Remington's Pharmaceutical Sciences. Edited by Gennaro AR, 19th ed. Pennsylvania: Mack Publishing Company, 1995) and includes, but is not limited to: pH adjusting agents, surfactants, adjuvants, ionic strength enhancers, diluents, agents to maintain osmotic pressure, agents to delay absorption, preservatives. For example, pH adjusting agents include, but are not limited to, phosphate buffered saline. Surfactants include, but are not limited to, cationic, anionic or non-ionic surfactants, such as Tween-80. Ionic strength enhancers include, but are not limited to, sodium chloride. Preservatives include, but are not limited to, various antibacterial and antifungal agents, such as parabens, trichloro-t-butanol, phenol, sorbic acid, and the like. Agents to maintain osmotic pressure include, but are not limited to, sugar, NaCl, and the like. Agents to delay absorption include, but are not limited to, monostearate and gelatin. Diluents include, but are not limited to, water, aqueous buffers (e.g., buffered saline), alcohols and polyols (e.g., glycerol), and the like. Preservatives include, but are not limited to, various antibacterial and antifungal agents, such as thimerosal, 2-phenoxyethanol, parabens, trichloro-t-butanol, phenol, sorbic acid, and the like. Stabilizers have the meaning commonly understood by those skilled in the art, which can stabilize the desired activity of active ingredient in drug, including but not limited to sodium glutamate, gelatin, SPGA, saccharides (e.g., sorbitol, mannitol, starch, sucrose, lactose, dextran, or glucose), amino acids (e.g., glutamic acid, glycine), proteins (e.g., dried whey, albumin, or casein) or degradation products thereof (e.g., lactalbumin hydrolysate), etc. In certain exemplary embodiments, the pharmaceutically acceptable carrier or excipient includes a sterile injectable liquid (e.g., an aqueous or non-aqueous suspension or solution). In certain exemplary embodiments, such sterile injectable liquid is selected from the group consisting of water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% polysorbate 20), pH buffer solution (e.g., phosphate buffer solution), Ringer's solution, and any combination thereof.

As used herein, the term “treatment/treating” refers to a method that is carried out in order to obtain a beneficial or desired clinical outcome. For the purpose of the disclosure, the beneficial or desired clinical outcome includes, but is not limited to, easing symptom, narrowing the scope of disease, stabilizing (i.e., not aggravating) the state of disease, delaying or slowing the progress of disease, and alleviating symptoms (either partially or completely), no matter detectable or not detectable. In addition, “treatment” also refers to a prolonged survival period compared to the expected survival period (if no treatment is accepted). In certain embodiments, the beneficial or desired clinical outcome described herein includes, but is not limited to, slowing of tumor progression, cancer regression, a reduction in tumor growth or size, a decrease in severity of at least one disease symptom, an increase in frequency and duration of disease symptom-free periods, a prevention of impairment or disability due to the disease affliction, or otherwise amelioration of disease symptoms in the patient.

As used herein, the term “subject” refers to any human or non-human animal that receives either prophylactic or therapeutic treatment. The term “non-human animal” includes all vertebrates, e.g., mammals and non-mammals, such as non-human primates, sheep, dog, cow, chickens, amphibians, reptiles, etc.

As used herein, the term “an effective amount” refers to an amount that is sufficient to achieve or at least partially achieve the expected effect. For example, an effective amount for treating a disease refers to an amount effective for curing or at least partially blocking a disease and its complication in a patient having the disease. The determination of such an effective amount is within the ability of a person skilled in the art. For example, an amount effective for a therapeutic use depends on severity of a disease to be treated, general state of the immune system in a patient, general conditions of a patient, such as age, weight and gender, administration routes of drugs, additional therapies used simultaneously, and the like.

As used herein, the term “effector function” refers to those biological activities attributable to the Fc region of an antibody (Fc region of a natural sequence or an amino acid sequence variant), which varies as the isotype of an antibody. Examples of antibody effector functions include, but are not limited to, Fc receptor binding affinity, antibody-dependent cell-mediated cytotoxicity (ADCC), complement dependent cytotoxicity (CDC), antibody-dependent cellular phagocytosis (ADCP), downregulation of cell surface receptors (e.g., B cell receptors), B cell activation, cytokine secretion, half-life/clearance of antibodies and antigen-antibody complexes, and the like. Methods for altering the effector function of an antibody are known in the art, for example by introducing a mutation in the Fc region.

As used herein, the term “antibody-dependent cell-mediated cytotoxicity (ADCC)” refers to a form of cytotoxicity, in which cytotoxic effector cells specifically bind to the target cells to which the antigen is attached, through the binding of Ig to an Fc receptor (FcR) presented on cytotoxic cells (e.g., natural killer (NK) cells, neutrophils or macrophages), and then kills the target cells by secreting cytotoxins. Methods for detecting ADCC activity of an antibody are known in the art.

As used herein, the term “antibody-dependent cellular phagocytosis (ADCP)” refers to a form of phagocytosis, in which antibody-opsonized target cells activate the FcγRs on the surface of effector cells with phagocytic potential, such as monocytes and macrophages, to induce phagocytosis, resulting in internalization and degradation of the target cell through phagosome acidification. Methods for detecting ADCP activity of an antibody are known in the art.

As used herein, the terms “cancer”, “tumor” and “proliferative disorder” are used interchangeably and refer to a large group of diseases characterized by the uncontrolled growth of abnormal cells in the body. Unregulated cell division may lead to the formation of malignant tumors or cells that invade adjacent tissues, and may metastasize to distant parts of the body through the lymphatic system or bloodstream. Cancers include benign and malignant cancers as well as dormant tumors or micrometastasis.

The terms “cancer”, “tumor” or “proliferative disorder” may include, without limitation, a solid cancer. A “solid cancer” includes, without limitation, breast cancer, ovarian cancer, head and neck cancer, bladder cancer, melanoma, colorectal cancer, pancreatic cancer, lung cancer, leiomyoma, leiomyosarcoma, glioma, glioblastoma, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

The terms “cancer”, “tumor” or “proliferative disorder” also includes, without limitation, a hematologic malignancy. A “hematologic malignancy” may be taken to refer to at least a blood cancer. Such cancers originate in blood-forming tissue, such as the bone marrow or other cells of the immune system. A hematologic malignancy includes, without limitation, leukemia (e.g., acute lymphocytic leukemia (ALL), acute myeloid leukemia (AML), chronic lymphocytic leukemia (CLL), chronic myelogenous leukemia (CML), Myeloproliferative disorder/neoplasm (MPDS), and myelodysplastic syndrome (MDS)), lymphoma (e.g., Hodgkin's lymphoma, both indolent and aggressive non-Hodgkin's lymphoma, Burkitt's lymphoma, and follicular lymphoma (small cell and large cell)), myeloma (e.g., multiple myeloma (MM), giant cell myeloma, heavy-chain myeloma, and light chain or Bence-Jones myeloma).

As used herein, the terms “about” when used to modify a numeric value or numeric range, indicate that deviations of up to 10% above (e.g., up to 5% above, up to 2% above, or up to 1% above) and up to 10% below (e.g., up to 5% below, up to 2% below, or up to 1% below) the value or range remain within the intended meaning of the recited value or range.

### Antibodies of the Disclosure

The inventors discovered that by reducing the binding affinity of anti-CD47 antibody the toxicities to RBCs were significantly reduced. Therefore, the inventors tuned the binding affinity of an anti-CD47 antibody to a certain level that the toxicities to cancer cells was maintained while the toxicities to RBCs were minimized, thereby generating the improved anti-CD47 antibodies provided herein showing better tumor growth inhibition but no obvious hemagglutination and phagocytosis of RBCs.

In one aspect, the disclosure provides an antibody or antigen-binding fragment thereof, that binds to CD47 (e.g., human CD47), comprising: HCDR1, HCDR2 and HCDR3 of the VH as set forth in SEQ ID NO: 23, 22, 27, 47, 49, 8, 9, 10, 11, 12, 13, 14, 15, 16, 17, 18, 19, 20, 21, 24, 25, 26, 37, 38, 39, 40, 41, 42, 43, 44, 45, 46, 48, 50, 51, or 52; and LCDR1, LCDR2 and LCDR3 of the VL as set forth in SEQ ID NO: 7 or 36.

In certain embodiments, the CDRs are defined according to Kabat, AbM, IMGT, or Chothia numbering system, or any combination thereof. In certain embodiments, the HCDR1 is defined according to AbM numbering system and HCDR2, HCDR3, LCDR1, LCDR2 and LCDR3 are defined according to Kabat numbering system.

### VH5-Series

In certain embodiments, the antibody or antigen-binding fragment thereof of the disclosure comprises: HCDR1, HCDR2 and HCDR3 of the VH as set forth in SEQ ID NO: 23, 22, 27, 8, 9, 10, 11, 12, 13, 14, 15, 16, 17, 18, 19, 20, 21, 24, 25, or 26; and LCDR1, LCDR2 and LCDR3 of the VL as set forth in SEQ ID NO: 7.

In certain embodiments, the antibody or antigen-binding fragment thereof comprises:
(i) HCDR1 comprising the sequence of GYX₁FTHHWX₂H (SEQ ID NO: 116), wherein X₁ is T or S, X₂ is M or I;
(ii) HCDR2 comprising the sequence of MIDASDX₃ETRYX₄QKFX₅G (SEQ ID NO: 117), wherein X₃ is K, I, or Y, X₄ is A or S, X₅ is Q or K;
(iii) HCDR3 comprising the sequence of SEQ ID NO: 90;
(iv) LCDR1 comprising the sequence of SEQ ID NO: 93;
(v) LCDR2 comprising the sequence of SEQ ID NO: 97; and,
(vi) LCDR3 comprising the sequence of SEQ ID NO: 99.

In certain embodiments, X₁ is T, and/or, X₂ is M. In certain embodiments, the HCDR1 comprises the sequence of SEQ ID NO: 57.

In certain embodiments, X₄ is A, and/or, X₅ is Q; preferably, the HCDR2 comprises the sequence of SEQ ID NO: 73, 72, or 77.

In certain embodiments, HCDR1, HCDR2 and HCDR3 comprising the amino acid sequences of SEQ ID NOs: 57, 73, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 72, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 77, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 65, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 66, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 68, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 69, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 70, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 71, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 74, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 75, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 57, 76, and 90, respectively; and LCDR1, LCDR2 and LCDR3 comprising the amino acid sequences of SEQ ID NOs: 93, 97, and 99, respectively. In certain embodiments, HCDR1, HCDR2 and HCDR3 comprising the amino acid sequences of SEQ ID NOs: 56, 65, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 56, 65, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 55, 62, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 54, 62, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 54, 65, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 54, 66, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 54, 67, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 53, 63, and 90, respectively; and LCDR1, LCDR2 and LCDR3 comprising the amino acid sequences of SEQ ID NOs: 93, 97, and 99, respectively.

In certain embodiments, HCDR1, HCDR2 and HCDR3 comprising the amino acid sequences of SEQ ID NOs: 53, 64, and 90, respectively; and LCDR1, LCDR2 and LCDR3 comprising the amino acid sequences of SEQ ID NOs: 93, 97, and 99, respectively.

In certain embodiments, the antibody or antigen-binding fragment thereof of the disclosure further comprising: four heavy chain framework regions (HFR1, HFR2, HFR3, and HFR4) comprising the amino acid sequences of SEQ ID NOs: 100, 101, 102 and 103, respectively; and/or, four light chain framework regions (LFR1, LFR2, LFR3, and LFR4) comprising the amino acid sequences of SEQ ID NOs: 108, 109, 110 and 111, respectively.

In certain embodiments, the antibody or antigen-binding fragment thereof of the disclosure comprising: a VH comprising the sequence of SEQ ID NO: 23 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 22 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 27 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 8, 9, 10, 11, 12, 13, 14, 15, 16, 17, 18, 19, 20, 21, 24, 25, or 26 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 7 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

### VH10-Series

In certain embodiments, the antibody or antigen-binding fragment thereof of the disclosure comprises: HCDR1, HCDR2 and HCDR3 of the VH as set forth in SEQ ID NO: 47, 49, 50, 51, 52, 37, 38, 39, 40, 41, 42, 43, 44, 45, 46, or 48; and LCDR1, LCDR2 and LCDR3 of the VL as set forth in SEQ ID NO: 36.

In certain embodiments, the antibody or antigen-binding fragment thereof comprises:
(i) HCDR1 comprising the sequence of GX₁X₂FX₃HHWIH (SEQ ID NO: 118), wherein X₁ is F or Y, X₂ is T or S, X₃ is S or T;
(ii) HCDR2 comprising the sequence of MIDASDSETRLX₄X₅X₆X₇KX₈ (SEQ ID NO: 119), wherein X₄ is S or V, X₅ is D or Q, X₆ is K or S, X₇ is F or V, X₈ is D or G;
(iii) HCDR3 comprising the sequence of SEQ ID NO: 90;
(iv) LCDR1 comprising the sequence of SEQ ID NO: 95;
(v) LCDR2 comprising the sequence of SEQ ID NO: 98; and,
(vi) LCDR3 comprising the sequence of SEQ ID NO: 99.

In certain embodiments, X₁ is F, X₂ is T, and/or, X₃ is S. In certain embodiments, the HCDR1 comprises the sequence of SEQ ID NO: 58.

In certain embodiments, X₄ is S, X₅ is D or Q, X₆ is K or S, X₇ is F or V, and X₈ is D or G.

In certain embodiments, X₄ is S or V, X₅ is D, X₆ is S, X₇ is F or V, and X₈ is G; preferably, X₄ is S and/or X₇ is F.

In certain embodiments, the HCDR2 comprises the sequence of SEQ ID NO: 84, 86, 87, 88, or 89.

In certain embodiments, HCDR1, HCDR2 and HCDR3 comprising the amino acid sequences of SEQ ID NOs: 58, 84, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 86, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 87, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 88, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 89, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 61, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 62, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 81, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 82, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 83, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 58, 85, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 53, 80, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 53, 78, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 53, 79, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 59, 61, and 90, respectively; and LCDR1, LCDR2 and LCDR3 comprising the amino acid sequences of SEQ ID NOs: 95, 98, and 99, respectively.

In certain embodiments, HCDR1, HCDR2 and HCDR3 comprising the amino acid sequences of SEQ ID NOs: 60, 61, and 90, respectively; and LCDR1, LCDR2 and LCDR3 comprising the amino acid sequences of SEQ ID NOs: 95, 98, and 99, respectively.

In certain embodiments, the antibody or antigen-binding fragment thereof of the disclosure further comprising: four heavy chain framework regions (HFR1, HFR2, HFR3, HFR4) comprising the amino acid sequences of SEQ ID NOs: 104, 105, 106 and 107, respectively; and/or, four light chain framework regions (LFR1, LFR2, LFR3, LFR4) comprising the amino acid sequences of SEQ ID NOs: 112, 113, 114 and 115, respectively.

In certain embodiments, the antibody or antigen-binding fragment thereof of the disclosure comprising: a VH comprising the sequence of SEQ ID NO: 47 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 49 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 50 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 51 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 52 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprising: a VH comprising the sequence of SEQ ID NO: 37, 38, 39, 40, 41, 42, 43, 44, 45, 46, or 48 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 36 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

### Constant Region

The antibody or antigen-binding fragment thereof of the present disclosure may further comprise a constant region sequence derived from a mammalian (e.g., murine or human) immunoglobulin.

In certain embodiments, the antibody or antigen-binding fragment thereof of the present disclosure comprise a constant region sequence derived from a human immunoglobulin.

Any immunoglobulin constant region can be used in the antibodies disclosed herein. In certain embodiments, the heavy chain constant region can be a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. Light chain constant region can be lambda or kappa. In certain embodiments, the heavy chain constant region is an IgG heavy chain constant region, such as an IgG1, IgG2, IgG3 or IgG4 heavy chain constant region. In certain embodiments, the light chain constant region is kappa.

The choice of constant region depends, in part, whether effector function (e.g., antibody-dependent cell-mediated cytotoxicity (ADCC), antibody-dependent cellular phagocytosis (ADCP) and/or complement-dependent cytotoxicity (CDC)) is desired. In certain embodiments, the heavy chain constant region with less or reduced effector function is preferred. In certain embodiments, the heavy chain constant region with effector function or enhanced effector function is preferred.

In certain embodiments, the heavy chain constant region with ADCC and/or ADCP or enhanced ADCC and/or ADCP is preferred.

In certain embodiments, the heavy chain of the antibody or antigen-binding fragment thereof of the present disclosure comprises a heavy chain constant region (CH) comprising an amino acid sequence derived from a human immunoglobulin heavy chain constant region.

In certain embodiments, the antibody or antigen-binding fragment thereof of the present disclosure comprises a heavy chain constant region that is a wild-type heavy chain constant region.

In certain embodiments, the antibody or antigen-binding fragment thereof of the present disclosure comprises a heavy chain constant region that is a variant of a wild-type heavy chain constant region. The constant region can be altered, e.g., mutated, to modify the properties of the antibody (e.g., to increase or decrease one or more of: stability, Fc receptor binding, antibody glycosylation, the number of cysteine residues, and/or effector function).

In certain embodiments, mutation(s) (e.g., one or more amino acid substitutions) can be introduced into the Fc region of constant region to increase stability or extend half-life of antibodies.

In certain embodiments, mutation(s) (e.g., one or more amino acid substitutions) can be introduced into the Fc region of constant region to alter (increase, reduce or eliminate) the effector function(s) of the antibody, such as ADCC, CDC or ADCP compared with the same antibody without the mutation(s).

In certain embodiments, the antibody or antigen-binding fragment thereof of the present disclosure comprises an IgG4 heavy chain constant region. In one embodiment, the IgG4 constant region is a wild-type constant region. In another embodiment, the IgG4 constant region comprises a mutation to alter the effector function(s), e.g., S228P to reduce Fab-arm exchange. In certain embodiments, the heavy chain constant region comprises or consists of the amino acid sequence of SEQ ID NO: 28.

In certain embodiments, the light chain of the antibody or antigen-binding fragment thereof of the present disclosure comprises a light chain constant region (CL) comprising an amino acid sequence derived from a human immunoglobulin light chain constant region.

In certain embodiments, the light chain constant region comprises or consists of the amino acid sequence of SEQ ID NO: 29.

### Full-Length Antibody

The antibody of the disclosure may be an antibody comprising two heavy chains and two light chains, having a conventional “Y” type structure.

In certain embodiments, the antibody or antigen-binding fragment thereof of the disclosure comprising a heavy chain and a light chain, wherein: the heavy chain comprises a VH comprising the sequence of SEQ ID NO: 23 and a CH comprising the sequence of SEQ ID NO: 28; and the light chain comprises a VL comprising the sequence of SEQ ID NO: 7 and a CL comprising the sequence of SEQ ID NO: 29.

In certain embodiments, the antibody or antigen-binding fragment thereof of the disclosure comprising a heavy chain and a light chain, wherein: the heavy chain comprises a VH comprising the sequence of SEQ ID NO: 22 and a CH comprising the sequence of SEQ ID NO: 28; and the light chain comprises a VL comprising the sequence of SEQ ID NO: 7 and a CL comprising the sequence of SEQ ID NO: 29.

In certain embodiments, the antibody or antigen-binding fragment thereof of the disclosure comprising a heavy chain and a light chain, wherein: the heavy chain comprises a VH comprising the sequence of SEQ ID NO: 27 and a CH comprising the sequence of SEQ ID NO: 28; and the light chain comprises a VL comprising the sequence of SEQ ID NO: 7 and a CL comprising the sequence of SEQ ID NO: 29.

In certain embodiments, the antibody or antigen-binding fragment thereof of the disclosure comprising a heavy chain and a light chain, wherein: the heavy chain comprises a VH comprising the sequence of SEQ ID NO: 47 and a CH comprising the sequence of SEQ ID NO: 28; and the light chain comprises a VL comprising the sequence of SEQ ID NO: 36 and a CL comprising the sequence of SEQ ID NO: 29.

In certain embodiments, the antibody or antigen-binding fragment thereof of the disclosure comprising a heavy chain and a light chain, wherein: the heavy chain comprises a VH comprising the sequence of SEQ ID NO: 49 and a CH comprising the sequence of SEQ ID NO: 28; and the light chain comprises a VL comprising the sequence of SEQ ID NO: 36 and a CL comprising the sequence of SEQ ID NO: 29.

### Antigen-Binding Fragment

The antibody of the present disclosure may also be a Fab fragment, Fab′, F(ab)₂, Fv, scFv, or any other type of fragment of an antibody having a conventional “Y” type structure, which substantially retains the ability to specifically bind to CD47 (e.g., human CD47) and the ability to inhibit, block, antagonize, neutralize or otherwise interfere with CD47 expression, activity and/or signaling, induce the phagocytosis of tumor cells, and inhibit the growth of tumors.

In certain embodiments, the antibody or antigen-binding fragment thereof of the disclosure is selected from the group consisting of scFv, Fab, Fab′, (Fab′)₂, Fv fragments, diabodies, bispecific antibodies, multispecific antibodies, or humanized antibodies.

In certain embodiments, the antibody or antigen-binding fragment thereof of the disclosure binds to wild-type human CD47 with a K_{D} of about 10⁻⁶ M about to 10⁻¹⁰ M, e.g., about 10⁻⁷ M about to 10⁻⁹ M, about 10⁻⁶ M about to 10⁻⁸ M, or about 10⁻⁷ M about to 10⁻⁸ M, or about 10⁻⁸ M about to 10⁻⁹ M.

In certain embodiments, the antibody or antigen-binding fragment thereof of the disclosure binds to human CD47 mutein with C33G mutation (e.g., set forth in SEQ ID NO: 1) with a K_{D} of about 10⁻⁶ M about to 10⁻¹⁰ M, e.g., about 10⁻⁷ M about to 10⁻⁹ M, about 10⁻⁶ M about to 10⁻⁸ M, or about 10⁻⁷ M about to 10⁻⁸ M, or about 10⁻⁸ M about to 10⁻⁹ M.

In certain embodiments, the antibody or antigen-binding fragment thereof of the disclosure inhibits, blocks, antagonizes, neutralizes or otherwise interferes with CD47 expression, activity and/or signaling, induces the phagocytosis of tumor cells, and inhibits the growth of the various hematological and solid tumors.

In certain embodiments, the antibody or antigen-binding fragment thereof of the disclosure induces no obvious hemagglutination and phagocytosis of RBCs.

In certain embodiments, the antibody or antigen-binding fragment thereof of the disclosure has improved efficacy to toxicity ratio.

Also provided are “conservative sequence modifications” to the antibody sequence provided herein, i.e., nucleotide and amino acid sequence modifications that do not abrogate the binding of the antibody encoded by the nucleotide sequence or containing the amino acid sequence, to the antigen. For example, modifications can be introduced by standard techniques known in the art, such as site-directed mutagenesis and PCR-mediated mutagenesis. The resulting modified antibodies can be screened for its binding activity.

Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). See the table below for examples of conservative substitution. Thus, a predicted nonessential amino acid residue in the antibodies disclosed herein could be preferably replaced with another amino acid residue from the same side chain family. Methods of identifying nucleotide and amino acid conservative substitutions that do not eliminate antigen binding are well-known in the art. See, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

Conservative Amino Acid Substitutions

| Amino Acid | Substitutions |
|---|---|
| A | S, G, C |
| R | K, Q, H |
| N | Q, H, E, D |
| D | E, N, Q |
| C | S, M, T |
| Q | N, K, E, D, R |
| E | D, N, Q |
| G | P, A, S |
| H | N, Q, K |
| I | L, V, M, A |
| L | I, V, M, A |
| K | R, Q, H |
| M | L, I, V, A, F |
| F | M, L, Y, W, H |
| S | T, C, A |
| T | S, V, A |
| W | Y, F |
| Y | W, F, H |
| V | I, L, M, A, T |

### Derivatized Antibodies

The antibody or an antigen-binding fragment thereof of the disclosure can be derivatized, for example, linked to another molecule (e.g., another polypeptide or protein). In general, the derivatization (such as labeling) of an antibody or an antigen-binding fragment thereof would not affect its binding to CD47 adversely. Therefore, the antibody or an antigen-binding fragment thereof of the disclosure is also intended to include such derivatized forms. For example, the antibody or an antigen-binding fragment thereof of the disclosure can be functionally linked (by chemical coupling, genetic fusion, non-covalent linkage or other means) to one or more other molecular groups, such as another antibody (e.g. forming a bispecific antibody), a detection agent, a medicinal agent, and/or a protein or polypeptide capable of mediating associate of the antibody or an antigen binding fragment thereof with another molecule (such as an avidin or a polyhistidine-tag).

### Labeled Antibodies

In certain embodiments, the antibody or antigen-binding fragment thereof of the present disclosure is labeled, such as a detectable label. The detectable label may be any substance that can be detected directly or indirectly, e.g., through an enzymatic reaction or molecular interaction. In certain embodiments, the label can be detected by fluorescent, spectroscopic, photochemical, biochemical, immunological, electrical, optical, or chemical means. In certain embodiments, the detectable label can be suitable for immunological detection (e.g., enzyme-linked immunoassay, radioimmunoassay, fluorescent immunoassay, chemiluminescence immunoassay, etc.). In certain embodiments, the label is selected from an enzyme, a radionuclide, a fluorescent dye, a luminescent substance (e.g., a chemiluminescent substance), or a biotin. In certain embodiments, the label can be linked to the antibody or antigen-binding fragment thereof of the disclosure via linkers of different lengths to reduce potential steric hindrance.

In certain embodiments, the labeled antibody or antigen-binding fragment thereof as described herein can be useful for detecting the presence of CD47 in a biological sample. The term “detecting” as used herein encompasses quantitative or qualitative detection. In certain embodiments, the biological sample is blood, serum or other liquid samples of biological origin. In certain embodiments, the biological sample comprises a cell or tissue.

In certain embodiments, there is provided a method of detecting CD47 in a cell, comprising contacting the cell with the labeled antibody or antigen-binding fragment thereof as described herein. In certain embodiments, a method of detecting the presence of CD47 in a biological sample is provided. In certain embodiments, the method comprises detecting the presence of CD47 protein in a biological sample. In certain embodiments, the CD47 is human CD47. In certain embodiments, the method comprises contacting the biological sample with the labeled antibody or antigen-binding fragment thereof as described herein under conditions permissive for binding of the antibody or antigen-binding fragment thereof to CD47, and detecting signal from the label. Such method may be an in vitro or in vivo method. In certain embodiments, there is provided a method of diagnosing a disease associated with CD47 expression (e.g., cancer) in an individual, comprising administering to the individual the labeled antibody or antigen-binding fragment thereof as described herein, and detecting the label in the individual. In certain embodiments, the labeled antibody or antigen-binding fragment thereof as described herein is used to select subjects eligible for therapy with any of anti-CD47 therapeutic agents (e.g., the antibody or antigen-binding fragment thereof, the immunoconjugate, or the bispecific or multispecific antibody as described herein), wherein CD47 is a biomarker for selection of patients.

### Immunoconjugates

In another aspect, there is provided an immunoconjugate comprising the antibody or antigen-binding fragment thereof as described herein and an effector molecule. Exemplary effector molecules include, but are not limited to, a drug, a toxin, a radioisotope, a protein, a peptide, and a nucleic acid. In certain embodiments, the effector molecule is a therapeutic agent.

In certain embodiments, the immunoconjugate of the disclosure comprises the antibody or antigen-binding fragment thereof as described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In certain embodiments, the immunoconjugate of the disclosure is an antibody-drug conjugate (ADC) in which the antibody or antigen-binding fragment thereof as described herein is conjugated to one or more drugs, including but not limited to a maytansinoid (see U.S. Pat. Nos. 5,208,020, 5,416,064 and European Patent EP0425235B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53: 3336-3342 (1993); and Lode et al., Cancer Res. 58: 2925-2928 (1998)); an anthracycline such as daunomycin or doxorubicin (see Kratz et al., Current Med. Chem. 13: 477-523 (2006); Jeffrey et al., Bioorganic & Med. Chem. Letters 16: 358-362 (2006); Torgov et al., Bioconj. Chem. 16: 717-721 (2005); Nagy et al., Proc. Natl. Acad. Sci. USA 97: 829-834 (2000); Dubowchik et al., Bioorg. & Med. Chem. Letters 12: 1529-1532 (2002); King et al., J. Med. Chem. 45: 4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, the immunoconjugate of the disclosure comprises the antibody or antigen-binding fragment thereof as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In certain embodiments, the immunoconjugate of the disclosure comprises the antibody or antigen-binding fragment thereof as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, “MRI”), such as iodine-123, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2, 6-diisocyanate), and bis-active fluorine compounds (such as 1, 5-difluoro-2, 4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52: 127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate) which are commercially available (e.g., from Pierce Biotechnology, Inc., Rockford, IL., U.S.A).

### Bispecific Antibodies

The antibody or antigen-binding fragment thereof of the disclosure can be used for forming bispecific or multispecific antibodies. The antibody or antigen-binding fragment thereof of the disclosure may be a part of a bispecific or multispecific antibody that includes a second functional module (e.g., a second antibody) having a binding specificity different from that of the antibody or antigen-binding fragment thereof of the disclosure, so that it is capable of binding to at least two different binding sites and/or target molecules. For example, the antibody or antigen-binding fragment thereof of the disclosure can be linked to a second antibody or antigen-binding fragment thereof that specifically binds to any protein that can be used as a potential target for combination therapy. To generate the bispecific or multispecific antibody, the antibody or antigen-binding fragment thereof of the disclosure can be linked (e.g., by chemical coupling, gene fusion, non-covalent association, or other means) to one or more other binding molecules (e.g., additional antibodies, antibody fragments, peptides, or binding mimics).

Accordingly, in another aspect, the disclosure provides a bispecific or multispecific antibody, comprising the antibody or antigen-binding fragment thereof of the disclosure.

In certain embodiments, the bispecific or multispecific antibody specifically binds to CD47 (e.g., human CD47) and a second target. In certain embodiments, the bispecific or multispecific antibody comprises a first antigen binding domain from the antibody or an antigen binding fragment thereof of the disclosure and a second antigen binding domain from an antibody against a second target.

In certain embodiments, the second target is an immunomodulatory receptor or tumor-associated antigen.

### Production of Antibodies

Antibodies disclosed herein can be obtained by genetic engineering recombination techniques. For example, DNA molecules of genes encoding the heavy and light chains of the antibodies of the disclosure can be obtained by chemical synthesis or PCR amplification. The resulting DNA molecule is inserted into an expression vector and then transfected into a host cell, such as HEK293 cell, CHO cell, or other cells that do not produce an immunoglobulin. Then, the transfected host cells are cultured under specific conditions and express the antibody of the present disclosure.

Antigen-binding fragments disclosed herein can be obtained by hydrolysis of an intact antibody molecule. Alternatively, these antigen-binding fragments can be produced directly from recombinant host cells (reviewed in Hudson, curr. Opin. Immunol. 11: 548-557 (1999); Little et al., Immunol. Today, 21: 364-370 (2000)). For example, the Fab′ fragment can be obtained directly from recombinant host cells; and the Fab′ fragments can be chemically coupled to form an F(ab′)₂ fragment (Carter et al., Bio/Technology, 10: 163-167 (1992)). Furthermore, the Fv, Fab or F(ab′)₂ fragments can also be isolated directly from a culture of recombinant host cells. Other techniques for preparing these antigen-binding fragments are well known to those of ordinary skill in the art.

In another aspect, the disclosure provides an isolated nucleic acid molecule, comprising a nucleotide sequence encoding the antibody or an antigen binding fragment thereof of the disclosure, or its heavy chain variable region and/or light chain variable region, or the bispecific or multispecific antibody of the disclosure.

In certain embodiments, the isolated nucleic acid molecule comprises a first nucleotide sequence and a second nucleotide sequence encoding the heavy chain variable region and the light chain variable region of the antibody or antigen-binding fragment thereof of the present disclosure, respectively.

In certain embodiments, the isolated nucleic acid molecule comprises a first nucleotide sequence and a second nucleotide sequence encoding the heavy chain and the light chain of the antibody or antigen-binding fragment thereof of the present disclosure, respectively.

In certain embodiments, the isolated nucleic acid molecule comprises different nucleotide sequences encoding different polypeptide chains of the bispecific or multispecific antibody respectively.

In another aspect, the disclosure provides a vector (e.g., a cloning vector or an expression vector), comprising the isolated nucleic acid molecule of the disclosure.

In certain embodiments, the vector of the disclosure is, for example, a plasmid, a cosmid, a phage, etc. In certain embodiments, the vector can express the antibody or an antigen-binding fragment thereof of the disclosure in a subject (for example, mammal, such as human).

In certain embodiments, the vector comprises a first nucleotide sequence and a second nucleotide sequence encoding the heavy chain variable region and the light chain variable region of the antibody or antigen-binding fragment thereof of the present disclosure, respectively. The first and second nucleotide sequences can be located on same or different vectors.

In certain embodiments, the vector comprises a first nucleotide sequence and a second nucleotide sequence encoding the heavy chain and the light chain of the antibody or antigen-binding fragment thereof of the present disclosure, respectively. The first and second nucleotide sequences can be located on same or different vectors.

In certain embodiments, the vector comprises different nucleotide sequences encoding different polypeptide chains of the bispecific or multispecific antibody respectively. The different nucleotide sequences can be located on same or different vectors.

In another aspect, the disclosure provides a host cell, comprising or is transformed with the isolated nucleic acid molecule of the disclosure or the vector of the disclosure. Such host cells include, but are not limited to, prokaryotic cell such as E. coli cell, and eukaryotic cell such as yeast cell, insect cell, plant cell and animal cell (e.g., mammalian cell, such as mouse cell and human cell).

In another aspect, provided is a method for producing the antibody or an antigen-binding fragment thereof of the disclosure, or the bispecific or multispecific antibody of the disclosure, comprising, culturing a host cell comprising the isolated nucleic acid molecule of the disclosure or the vector of the disclosure, or the host cell of the disclosure under a condition allowing expression of the antibody or an antigen-binding fragment thereof or the bispecific or multispecific antibody, and recovering the antibody or an antigen-binding fragment thereof or the bispecific or multispecific antibody from a culture of the cultured host cell.

### Pharmaceutical Composition

In one aspect, the disclosure provides a pharmaceutical composition, comprising the antibody or antigen-binding fragment thereof of the disclosure, or the immunoconjugate of the disclosure, the bispecific or multispecific antibody of the disclosure, or the isolated nucleic acid molecule, vector, or host cell of the disclosure, and a pharmaceutically acceptable carrier and/or excipient.

In certain embodiments, the pharmaceutical composition comprises the antibody or antigen-binding fragment thereof of the disclosure. In certain embodiments, the pharmaceutical composition comprises an effective amount of the antibody or antigen-binding fragment thereof of the disclosure. In certain embodiments, the antibody or antigen-binding fragment thereof is the only active ingredient included in the pharmaceutical composition.

In certain embodiments, the pharmaceutical composition comprises the immunoconjugate of the disclosure. In certain embodiments, the pharmaceutical composition comprises an effective amount of the immunoconjugate of the disclosure. In certain embodiments, the immunoconjugate is the only active ingredient included in the pharmaceutical composition.

In certain embodiments, the pharmaceutical composition comprises the bispecific or multispecific antibody of the disclosure. In certain embodiments, the pharmaceutical composition comprises an effective amount of the bispecific or multispecific antibody of the disclosure. In certain embodiments, the bispecific or multispecific antibody is the only active ingredient included in the pharmaceutical composition.

In certain embodiments, the pharmaceutical composition of the disclosure may further comprise an additional therapeutic agent. In certain embodiments, the additional therapeutic agent is an anti-tumor agent.

In certain embodiments, the additional therapeutic agent is an additional therapeutic antibody for cancer treatment, e.g, a therapeutic antibody with ADCC and/or ADCP activity or enhanced ADCC and/or ADCP activity. In certain embodiments, the additional therapeutic antibody binds to a target other than CD47. In certain embodiments, the target is selected from an immunomodulatory receptor or tumor-associated antigen.

In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide.

In certain embodiments, the antibody or antigen-binding fragment thereof of the disclosure or the immunoconjugate of the disclosure or the bispecific or multispecific antibody of the disclosure, and the additional therapeutic agent may be provided as separate components or as components of a single composition. The antibody or the antigen-binding fragment thereof of the disclosure or the immunoconjugate of the disclosure or the bispecific or multispecific antibody of the disclosure may be used in combination with the other agents simultaneously, separately, or successively.

The pharmaceutical composition can be provided in unit dosage form (i.e., the dosage for a single administration).

The pharmaceutical composition can be formulated using one or more pharmaceutically acceptable carriers and/or excipients. The formulation depends on the route of administration chosen. For parenteral administration, the pharmaceutical composition is preferably sterile and substantially isotonic and manufactured under GMP conditions. By way of example, the antibodies disclosed herein can be formulated in aqueous solutions for injection, preferably in physiologically compatible buffers, such as water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% polysorbate 20), pH buffered solution (e.g., phosphate buffered solution), Ringer's solution. The solution can contain formulatory agents such as suspending, stabilizing and/or dispersing agents. Alternatively, antibodies can be in lyophilized form for constitution with a suitable vehicle, e.g., sterile pyrogen-free water, before use.

Pharmaceutical compositions described herein can be useful in inducing phagocytosis of cancer cells and treating a cancer or proliferative disorder while inducing no obvious hemagglutination and phagocytosis of RBCs.

### Uses and Methods

The antibody or antigen-binding fragment thereof of the present disclosure can specifically bind to CD47 (e.g., human CD47), inhibit, block, antagonize, neutralize or otherwise interfere with CD47 expression, activity and/or signaling, thereby inducing the phagocytosis of tumor cells and inhibiting the growth of the various hematological and solid tumors. Meanwhile, the antibody or antigen-binding fragment thereof disclosed herein induces no obvious hemagglutination and phagocytosis of RBCs, exerting a minimal adverse effect on RBCs to avoid severe anemia. Accordingly, the antibodies described herein may be used in a treatment in a wide variety of therapeutic applications, including, treating cancers or proliferative disorders.

In one aspect, the disclosure provides a method of treating a cancer or proliferative disorder in a subject in a subject (e.g., human), comprising administering to a subject in need thereof an effective amount of the antibody or antigen-binding fragment thereof, immunoconjugate, bispecific or multispecific antibody, isolated nucleic acid molecule, vector, host cell, or pharmaceutical composition disclosed herein.

In another aspect, the disclosure provides use of the antibody or antigen-binding fragment thereof, immunoconjugate, bispecific or multispecific antibody, isolated nucleic acid molecule, vector, host cell, or pharmaceutical composition disclosed herein in the manufacture of a medicament for use in treating a cancer or proliferative disorder in a subject.

In another aspect, the disclosure relates to the antibody or antigen-binding fragment thereof, immunoconjugate, bispecific or multispecific antibody, isolated nucleic acid molecule, vector, host cell, or pharmaceutical composition disclosed herein for use in treating a cancer or proliferative disorder in a subject.

In certain embodiments, in any of the methods or uses of treatment described herein, the antibody or antigen-binding fragment thereof of the disclosure inhibits, blocks, antagonizes, neutralizes or otherwise interferes with CD47 expression, activity and/or signaling, induces the phagocytosis of tumor cells, and inhibits the growth of the various hematological and solid tumors.

In certain embodiments, in any of the methods or uses of treatment described herein, the antibody or antigen-binding fragment thereof of the disclosure induces no obvious hemagglutination and phagocytosis of RBCs.

In certain embodiments, in any of the methods or uses of treatment described herein, the antibody or antigen-binding fragment thereof of the disclosure has improved efficacy to toxicity ratio.

In certain embodiments, the cancer or proliferative disorder involved in the methods or uses of treatment described herein, is associated with CD47 and/or SIRPα expression. In certain embodiments, the cancer or proliferative disorder is associated with CD47 expression.

In certain embodiments, the cancer or proliferative disorder involved in the methods or uses of treatment described herein includes, without limitation, solid tumors and hematological malignancies.

In certain embodiments, the cancer or proliferative disorder is a hematological cancer, e.g., leukemia, lymphoma or myeloma. In certain embodiments, the hematological cancer is a leukemia selected from the group consisting of acute lymphocytic leukemia (ALL), acute myeloid leukemia (AML), chronic lymphocytic leukemia (CLL), chronic myelogenous leukemia (CML), Myeloproliferative disorder/neoplasm (MPDS), and myelodysplastic syndrome (MDS). In certain embodiments, the hematological cancer is a lymphoma selected from the group consisting of a Hodgkin's lymphoma, both indolent and aggressive non-Hodgkin's lymphoma, Burkitt's lymphoma, and follicular lymphoma (small cell and large cell). In certain embodiments, the hematological cancer is a myeloma selected from the group consisting of multiple myeloma (MM), giant cell myeloma, heavy-chain myeloma, and light chain or Bence-Jones myeloma.

In certain embodiments, the cancer or proliferative disorder is a solid tumor, e.g., breast cancer, ovarian cancer, head and neck cancer, bladder cancer, melanoma, colorectal cancer, pancreatic cancer, lung cancer, leiomyoma, leiomyosarcoma, glioma, glioblastoma, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

In certain embodiments, in any of the methods or uses of treatment described herein, the antibody, immunoconjugate or bispecific or multispecific antibody of the disclosure can be used alone. Alternatively, the antibody, immunoconjugate or bispecific or multispecific antibody of the disclosure can be used in combination with additional therapeutic agent. In certain embodiments, the additional therapeutic agent is an anti-tumor agent.

In certain embodiments, the additional therapeutic agent is an additional therapeutic antibody for cancer treatment, e.g, a therapeutic antibody with ADCC and/or ADCP activity or enhanced ADCC and/or ADCP activity. In certain embodiments, the additional therapeutic antibody binds to a target other than CD47. In certain embodiments, the target is selected from an immunomodulatory receptor or tumor-associated antigen.

In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide.

In certain embodiments, in any of the methods or uses of treatment described herein, the antibody, immunoconjugate or bispecific or multispecific antibody of the disclosure is used in combination with an additional therapy (e.g, standard cancer treatment, such as surgery, chemotherapy, radiation therapy, targeted therapy, immunotherapy, hormone therapy, gene therapy or palliative care).

In certain embodiments, in any of the methods or uses of treatment described herein, the antibody or antigen-binding fragment thereof, the immunoconjugate, the bispecific or multispecific antibody, or pharmaceutical composition of the present disclosure can be formulated into any dosage form known in the medical field, for example, tablets, pills, suspensions, emulsions, solutions, gels, capsules, powders, granules, elixirs, lozenges, suppositories, injections (including injection liquids, sterile powders for injection and concentrated solutions for injection), inhalants, sprays, etc. The preferred dosage form depends on the intended route of administration and therapeutic use. One preferred dosage form is an injection. Such injection may be a sterile injectable solution. Alternatively, the sterile injectable solution can be prepared as a sterile lyophilized powder (e.g., by vacuum drying or freeze drying) for the convenience of storage and use.

In certain embodiments, in any of the methods or uses of treatment described herein, the antibody or antigen-binding fragment thereof, the immunoconjugate, the bispecific or multispecific antibody, or pharmaceutical composition of the present disclosure can be administrated by any suitable method known in the art, including, but not limited to, oral, buccal, sublingual, eyeball, topical, parenteral, rectal, intrathecal, intracytoplasmic, groin, intravesical, local (e.g., powder, ointment or drops), or nasal route. However, for many therapeutic uses, the preferred route/mode of administration is parenteral administration (e.g., intravenous injection or bolus, subcutaneous injection, intraperitoneal injection, intramuscular injection). The skilled person will understand that the route and/or mode of administration will vary depending on the intended purpose. In a preferred embodiment, the antibodies disclosed herein are given by intravenous injection or bolus.

In certain embodiments, in any of the methods or uses of treatment described herein, the subject is a human. In certain embodiments, the subject has a cancer.

### EXAMPLES

The following examples discuss the humanization and optimization of monoclonal antibodies against human CD47 and also provide exemplary methods by which the activities of binding, blocking, inducing phagocytosis of RBCs and RBC hemagglutination, and tumor growth inhibition of the antibodies described in this application can be determined.

The examples provided below are for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### Example 1: Optimization of Anti-Human CD47 Antibody

Humanization, pioneered by Winter and colleagues (Winter G, Harris W J 1993), takes advantage of the conserved nature of the antibody frameworks that allow for grafting of the murine CDRs onto a human acceptor framework. Strategies to select a human acceptor framework include using the human germline gene that is most closely related to the parent murine antibody or using a well-behaved “fixed framework”. We used both strategies for the humanization and optimization of a mouse anti-human CD47 antibody.

### Closely Related Human Germline Gene as the Sequence Acceptor

The VH (hereafter referred to as 108VH, SEQ ID NO: 2) and VL (hereafter referred to as 108VL, SEQ ID NO: 3) sequences of mouse anti-CD47 antibody 108C10A6 (WO 2019/144895 A1) were analyzed. The humanization of 108VL was based on the humanized VL sequence of 108VL1.M1 (SEQ ID NO: 5). Germlining was carried out so that more murine CDR residues were changed to their human germline counterparts, generating the sequence of 108VL1.M5 (SEQ ID NO: 7). The most appropriate human VH frameworks on which to build the CDR grafted heavy chain were identified to be those of GenBank accession #ABM67212. Straight grafting of 108VH CDRs onto ABM67212 human frameworks generated the humanized VH sequence of 108VH5 (SEQ ID NO: 6). No backmutation in the framework region was carried out. To remove the potential acid labile Asp52-Pro53 motif in HCDR2, P53A mutation was introduced to the humanized VH sequence. Also, murine HCDR residues were gradually mutated to their human germline counterparts to increase the humanness of the sequences, generating the sequences of 108VH5.M5 (SEQ ID NO: 8), 108VH5.M6 (SEQ ID NO: 9), 108VH5.M8-108VH5.M12 (SEQ ID NOs: 10-14), 108VH5.M14-108VH5.M16 (SEQ ID NOs: 15-17). To remove the potential isomerization of Asp55-Gly56 motif in HCDR2 of 108VH5.M16, mutations of Asp55 and Gly56 were carried out, generating the sequences of 108VH5.M17 (SEQ ID NO: 18), 108VH5.M18 (SEQ ID NO: 19), 108VH5.M20-108VH5.M27 (SEQ ID NOs: 20-27). These humanized VH and VL sequences were connected to human IgG4 constant region sequence with S228P mutation (SEQ ID NO: 28) and human κ light chain constant region sequence (SEQ ID NO: 29), respectively, to construct full-length IgGs. Heavy chain and light chain plasmids were prepared, paired and used for IgG production by HEK293 cells. Supernatants of transfected HEK293 cells were collected and subjected to surface plasmon resonance (SPR) assay on a BIAcore T200 instrument (GE Healthcare).

In the sequence of the IgV domain of CD47, the first cysteine residue, Cys33 was confirmed to form a disulfide bond with Cys263 of the multiply membrane-spanning (MMS) domain (Rebres R A 2001). The existence of the free Cys33 in the IgV domain may induce the formation of CD47 IgV domain homodimer. Therefore, in addition to the wild type human CD47 (Acro Biosystems, cat. no. CD7-H5227), CD47 mutein with C33G mutation (hereafter referred to as CD47-C33G) was also used for the determination of true mono-valent binding affinity. The SPR assay was carried out as follows: antibodies secreted to the medium were captured onto the sensorchip pre-coated with goat-anti-human pAb (Jackson ImmunoResearch, cat. no. 109-005-098) through the interaction between polyclonal antibody and human Fc. Increasing concentrations (ranging from 2.5 nM to 1.28 μM) of wild-type CD47 or CD47-C33G flowed over the sensorchip surface, and were allowed to bind the captured antibody for 100 s followed by injection of the running buffer to allow dissociation of the antigen proteins. On-rate (kₐ) and off-rate (k_{d}) were calculated based on association and dissociation curve, and were used to estimate the equilibrium dissociation constant (K_{D}). The binding affinities of chimeric antibody 108C10A6 and previously humanized antibodies 108VH4.M4-VL1.M1 (WO 2019/144895 A1, variable domain SEQ ID NOs: 373 and 377) and TJC4 positive control antibody (US 2020/0140565 A1, variable domain SEQ ID NOs: 31 and 32) were also measured for comparison.

As can be expected, for antibodies with lower mono-valent binding affinity, the difference between binding affinities measured using wild-type CD47 and CD47-C33G is pronounced, especially for antibodies with μM or sub μM K_{D}. After humanization, a series of humanized antibodies with binding affinity ranging from 1.3 μM to 3.4 nM were generated (Table 1).

TABLE 1
Humanization of 108C10A6 using the frameworks of closest related human germline sequences.

| experiment | Antibody | | Human CD47 wt | | | Human CD47-C33G | | |
|---|---|---|---|---|---|---|---|---|
| # | VH | VL | kₐ (1/Ms) | k_{d} (1/s) | K_{D} (M) | kₐ (1/Ms) | k_{d} (1/s) | K_{D} (M) |
| 1 | VH | VL | 1.5E+06 | 3.2E−04 | 2.0E−10 | 7.4E+05 | 6.8E−04 | 9.2E−10 |
| | VH4.M4 | VL1.M1 | 1.1E+06 | 4.2E−04 | 4.0E−10 | 4.8E+05 | 8.7E−04 | 1.8E−09 |
| | VH5 | VL1.M5 | 7.8E+05 | 6.5E−04 | 8.3E−10 | 3.4E+05 | 1.2E−03 | 3.4E−09 |
| | VH5.M5 | VL1.M5 | 7.2E+05 | 9.6E−04 | 1.3E−09 | 3.3E+05 | 2.1E−03 | 6.2E−09 |
| | VH5.M6 | VL1.M5 | 4.9E+05 | 3.9E−03 | 8.0E−09 | 1.2E+05 | 9.7E−02 | 8.0E−07 |
| | VH5.M8 | VL1.M5 | 7.2E+05 | 2.2E−03 | 3.0E−09 | 3.0E+05 | 4.4E−03 | 1.5E−08 |
| | VH5.M9 | VL1.M5 | 7.6E+05 | 1.4E−03 | 1.9E−09 | 3.3E+05 | 2.8E−03 | 8.5E−09 |
| | TJC4 | | 5.0E+05 | 2.2E−03 | 4.5E−09 | 1.9E+05 | >1.0E−01* | >5.3E−07* |
| 2 | VH | VL | 1.6E+06 | 3.2E−04 | 1.9E−10 | 7.2E+05 | 7.3E−04 | 1.0E−09 |
| | VH5 | VL1.M5 | 7.2E+05 | 5.9E−04 | 8.2E−10 | 3.1E+05 | 1.2E−03 | 3.9E−09 |
| | VH5.M10 | VL1.M5 | 6.6E+05 | 1.9E−03 | 2.8E−09 | 2.5E+05 | 4.0E−03 | 1.6E−08 |
| | VH5.M11 | VL1.M5 | 2.6E+05 | 3.2E−03 | 1.2E−08 | 1.9E+05 | >1.0E−01* | >5.3E−07* |
| | VH5.M12 | VL1.M5 | 7.3E+05 | 2.0E−03 | 2.7E−09 | 2.8E+05 | 4.1E−03 | 1.5E−08 |
| | VH5.M14 | VL1.M5 | 6.9E+05 | 1.8E−03 | 2.6E−09 | 2.6E+05 | 3.7E−03 | 1.4E−08 |
| | VH5.M15 | VL1.M5 | 2.2E+05 | 4.8E−03 | 2.2E−08 | 1.6E+05 | >1.0E−01* | >6.3E−07* |
| | TJC4 | | 5.5E+05 | 2.4E−03 | 4.4E−09 | 1.7E+05 | 9.0E−02 | 5.4E−07 |
| 3 | VH | VL | 1.6E+06 | 3.2E−04 | 2.0E−10 | 6.6E+05 | 6.3E−04 | 9.5E−10 |
| | VH4.M4 | VL1.M1 | 1.0E+06 | 4.1E−04 | 4.1E−10 | 4.3E+05 | 8.4E−04 | 1.9E−09 |
| | VH5.M16 | VL1.M5 | 7.2E+05 | 1.9E−03 | 2.6E−09 | 2.9E+05 | 4.1E−03 | 1.4E−08 |
| | VH5.M17 | VL1.M5 | 4.6E+05 | 8.4E−03 | 1.8E−08 | 6.4E+04 | 8.4E−02 | 1.3E−06 |
| | VH5.M18 | VL1.M5 | 1.7E+05 | 2.0E−03 | 1.2E−08 | 7.5E+04 | 8.7E−02 | 1.2E−06 |
| | TJC4 | | 4.6E+05 | 2.2E−03 | 4.7E−09 | 1.6E+05 | >1.0E−01* | >6.3E−07* |
| 4 | VH | VL | 1.8E+06 | 3.0E−04 | 1.7E−10 | 9.2E+05 | 6.1E−04 | 6.7E−10 |
| | VH4.M4 | VL1.M1 | 1.1E+06 | 4.1E−04 | 3.8E−10 | 5.7E+05 | 8.6E−04 | 1.5E−09 |
| | VH5.M20 | VL1.M5 | 7.1E+05 | 2.2E−03 | 3.0E−09 | 3.1E+05 | 4.5E−03 | 1.5E−08 |
| | VH5.M21 | VL1.M5 | 4.9E+05 | 3.2E−03 | 6.5E−09 | 1.6E+05 | 8.0E−03 | 5.1E−08 |
| | VH5.M22 | VL1.M5 | 4.8E+05 | 2.9E−03 | 6.1E−09 | 2.0E+05 | 6.8E−03 | 3.5E−08 |
| | VH5.M23 | VL1.M5 | 4.4E+05 | 2.8E−03 | 6.4E−09 | 1.7E+05 | 5.8E−03 | 3.3E−08 |
| | VH5.M24 | VL1.M5 | 5.2E+05 | 3.0E−03 | 5.7E−09 | 2.2E+05 | 6.7E−03 | 3.1E−08 |
| | VH5.M25 | VL1.M5 | 4.5E+05 | 2.8E−03 | 6.2E−09 | 1.9E+05 | 6.3E−03 | 3.3E−08 |
| | VH5.M26 | VL1.M5 | 5.7E+05 | 2.6E−03 | 4.6E−09 | 2.4E+05 | 5.4E−03 | 2.3E−08 |
| | VH5.M27 | VL1.M5 | 4.9E+05 | 3.6E−03 | 7.4E−09 | 2.2E+05 | 8.5E−03 | 3.9E−08 |

| | | | | | | |
|---|---|---|---|---|---|---|
| TJC4 | 3.8E+05 | 1.8E−03 | 4.7E−09 | 2.1E+05 | 1.3E−01 | 6.0E−07 |

*The fitted value is out of the limit of the instrument, an estimated value was given.

### Humanized 4D5 Frameworks as the Sequence Acceptor

The humanized 4D5 frameworks had been optimized with the consensus sequence approach and the robust stability of the 4D5 scFv had been demonstrated (Wöm and Plückthun, 1999), therefore were used as the sequence acceptor on which the CDRs of 108VL1.M1 and 108VH were straightly grafted, generating the sequences of 108VL10.M1 (SEQ ID NO: 30) and 108VH10 (SEQ ID NO: 31), respectively. SPR affinity measurement was carried out on the straight-graft antibody as described above. The mono-valent binding affinity dropped by 34-fold (Table 2, experiment 1). Germlining was carried out on 108VL10.M1 and more murine CDR residues were changed to their human counterparts in the sequence of the closest human Vκ germline IGKV1-39*01, generating the sequence of 108VL10.Ma-108VL10.Md (SEQ ID NOs: 32-35). Similarly, murine VH CDR residues were changed to their human counterparts in the sequence of the closest human VH germline IGHV3-7*01, generating the sequence of 108VH10.Ma-108VH10.Mf (SEQ ID NOs: 37-42). From SPR experiment 2, none of the VL germlining mutations significantly reduced the mono-valent binding affinity of straight-graft antibody, except 108VL10.Mb which reduced the binding affinity by 7.7-fold. Therefore, the germlining mutations of 108VL10.Ma, 108VL10.Mc and 108VL10.Md were combined to further increase the humanness of VL, generating the sequence of 108VL10.Me (SEQ ID NO: 36). For VH germlining, 108VH10.Ma did not affect the binding affinity, therefore was kept in the humanized sequence. 108VH10.Mb did not affect the binding affinity either, and yet it might increase the risk of oxidation. 108VH10.Mc, 108VH10.Md and 108VH10.Me either significantly reduced or completely abolished the binding to human CD47-C33G, therefore were not incorporated in the humanized VH sequence. 108VH10.Mf reduced the mono-valent binding affinity by 6.7-fold compared to 108VH10 (Table 2, experiment 2). Now that the attempt to remove the potential acid labile Asp52-Pro53 motif in HCDR2 by replacing Asp52 with Lys (108VH10.Md) or Pro53 with Gln (108VH10.Me) did not succeed, P53A mutation was introduced to the humanized 108VH10.Ma sequence, generating the sequence of 108VH10.Mg (SEQ ID NO: 43). Further single-site germlining mutations were incorporated in 108VH10.Mg sequence, generating the sequences of 108VH10.Mh-108VH10.Ml (SEQ ID NOs: 44-48). 108VH10.Mg-108VH10.Ml were paired with 108VL10.Me to generate humanized antibodies that are more human. The binding affinities of these antibodies were shown in experiment 3, Table 2. Compared to 108VH10.Mg, 108VH10.Mi, 108VH10.Mj and 108VH10.Ml did not affect the binding affinity at all, therefore were combined to generate the sequence of 108VH10.Mo (SEQ ID NO: 49). Two germlining mutations S61V, F64V and combination thereof were incorporated to 108VH10.Mo, generating the sequences of 108VH10.Mp (SEQ ID NO: 50), 108VH10.Mq (SEQ ID NO: 51) and 108VH10.Mr (SEQ ID NO: 52), respectively. With these mutations, the mono-valent binding affinity was further reduced to ˜170 nM (experiment 4).

TABLE 2
Humanization of 108C10A6 using humanized 4D5 frameworks as the sequence acceptor.

| experiment | Antibody | | Human CD47 wt | | | Human CD47-C33G | | |
|---|---|---|---|---|---|---|---|---|
| # | VH | VL | kₐ (1/Ms) | k_{d} (1/s) | K_{D} (M) | kₐ (1/Ms) | k_{d} (1/s) | K_{D} (M) |
| 1 | VH | VL | 1.6E+06 | 3.2E−04 | 1.9E−10 | 7.2E+05 | 7.3E−04 | 1.0E−09 |
| | VH4.M4 | VL1.M1 | 1.0E+06 | 3.9E−04 | 3.9E−10 | 4.1E+05 | 8.7E−04 | 2.1E−09 |
| | VH10 | VL10.M1 | 2.3E+05 | 1.3E−03 | 5.6E−09 | 9.9E+04 | 3.4E−03 | 3.4E−08 |
| | TJC4 | | 5.5E+05 | 2.4E−03 | 4.4E−09 | 1.7E+05 | 9.0E−02 | 5.4E−07 |
| 2 | VH | VL | 1.6E+06 | 3.4E−04 | 2.1E−10 | 5.9E+05 | 7.4E−04 | 1.3E−09 |
| | VH4.M4 | VL1.M1 | 9.8E+05 | 4.6E−04 | 4.7E−10 | 3.8E+05 | 9.7E−04 | 2.6E−09 |
| | VH10 | VL10.M1 | 2.8E+05 | 1.6E−03 | 5.8E−09 | 8.7E+04 | 3.8E−03 | 4.3E−08 |
| | VH10 | VL10.Ma | 1.0E+06 | 1.5E−03 | 1.4E−09 | 3.6E+05 | 2.9E−03 | 8.0E−09 |
| | VH10 | VL10.Mb | 2.9E+05 | 1.1E−02 | 3.8E−08 | 4.7E+04 | 1.6E−02 | 3.3E−07 |
| | VH10 | VL10.Mc | 1.9E+05 | 2.0E−03 | 1.0E−08 | 6.9E+04 | 4.4E−03 | 6.4E−08 |
| | VH10 | VL10.Md | 2.3E+05 | 1.6E−03 | 7.1E−09 | 8.6E+04 | 3.6E−03 | 4.2E−08 |
| | VH10.Ma | VL10.M1 | 2.3E+05 | 2.0E−03 | 8.7E−09 | 8.2E+04 | 4.6E−03 | 5.7E−08 |
| | VH10.Mb | VL10.M1 | 2.4E+05 | 1.6E−03 | 6.5E−09 | 8.9E+04 | 3.5E−03 | 3.9E−08 |
| | VH10.Mc | VL10.M1 | 2.5E+05 | 5.4E−03 | 2.1E−08 | 6.1E+04 | >1.0E−01* | >1.6E−06* |
| | VH10.Md | VL10.M1 | Low Binding | | | No binding | | |
| | VH10.Me | VL10.M1 | 5.2E+04 | 2.7E−03 | 5.3E−08 | Low Binding | | |
| | VH10.Mf | VL10.M1 | 2.3E+05 | 7.1E−03 | 3.0E−08 | 4.9E+04 | 1.4E−02 | 2.9E−07 |
| | TJC4 | | 5.4E+05 | 2.4E−03 | 4.3E−09 | 4.9E+05 | >1.0E−07* | >2.0E−07* |
| 3 | VH | VL | 1.6E+06 | 3.2E−04 | 2.0E−10 | 6.6E+05 | 6.3E−04 | 9.5E−10 |
| | VH4.M4 | VL1.M1 | 1.0E+06 | 4.1E−04 | 4.1E−10 | 4.3E+05 | 8.4E−04 | 1.9E−09 |
| | VH10 | VL10.M1 | 2.3E+05 | 1.4E−03 | 6.1E−09 | 8.5E+04 | 3.0E−03 | 3.6E−08 |
| | VH10.Mg | VL10.Me | 6.8E+05 | 3.2E−03 | 4.8E−09 | 2.3E+05 | 7.2E−03 | 3.1E−08 |
| | VH10.Mh | VL10.Me | 3.8E+05 | 6.6E−03 | 1.7E−08 | 7.9E+04 | 1.3E−02 | 1.6E−07 |
| | VH10.Mi | VL10.Me | 6.4E+05 | 3.4E−03 | 5.2E−09 | 2.3E+05 | 8.7E−03 | 3.8E−08 |
| | VH10.Mj | VL10.Me | 6.1E+05 | 3.5E−03 | 5.8E−09 | 2.1E+05 | 7.8E−03 | 3.8E−08 |
| | VH10.Mk | VL10.Me | 9.5E+05 | 8.6E−03 | 9.0E−09 | 2.1E+05 | 1.9E−02 | 8.7E−08 |
| | VH10.Ml | VL10.Me | 6.1E+05 | 3.4E−03 | 5.6E−09 | 2.0E+05 | 7.3E−03 | 3.6E−08 |
| | TJC4 | | 4.6E+05 | 2.2E−03 | 4.7E−09 | 1.6E+05 | >1.0E−01* | >6.3E−07* |
| 4 | VH | VL | 1.8E+06 | 3.0E−04 | 1.7E−10 | 9.2E+05 | 6.1E−04 | 6.7E−10 |
| | VH4.M4 | VL1.M1 | 1.1E+06 | 4.1E−04 | 3.8E−10 | 5.7E+05 | 8.6E−04 | 1.5E−09 |
| | VH10 | VL10.M1 | 2.4E+05 | 1.3E−03 | 5.2E−09 | 1.1E+05 | 3.0E−03 | 2.8E−08 |
| | VH10.Mo | VL10.Me | 5.6E+05 | 3.2E−03 | 5.8E−09 | 2.1E+05 | 9.3E−03 | 4.5E−08 |
| | VH10.Mp | VL10.Me | 2.5E+05 | 3.9E−03 | 1.5E−08 | 7.2E+04 | 1.2E−02 | 1.7E−07 |
| | VH10.Mq | VL10.Me | 6.0E+05 | 4.5E−03 | 7.5E−09 | 1.9E+05 | 2.4E−02 | 1.2E−07 |
| | VH10.Mr | VL10.Me | 3.6E+05 | 3.1E−03 | 8.8E−09 | 1.5E+05 | 2.5E−02 | 1.6E−07 |

| | | | | | | |
|---|---|---|---|---|---|---|
| TJC4 | 3.8E+05 | 1.8E−03 | 4.7E−09 | 2.1E+05 | 1.3E−01 | 6.0E−07 |

*The fitted value is out of the limit of the instrument, an estimated value was given.

### Example 2: Production of Anti-CD47 Humanized Antibodies

Briefly, the plasmids of humanized antibodies were prepared and used to transfect CHO cells. Supernatants were collected 8 days after transfection. Antibodies secreted were purified by Protein A affinity chromatography and size exclusion chromatography. As positive controls, two anti-CD47 antibodies TJC4 and Hu5F9 (US 2015/0183874 A1, variable domain SEQ ID NOs: 37 and 42), and TTI-622 fusion protein (U.S. Ser. No. 10/906,954 B2, SEQ ID NOs: 26) were also produced in house according to sequences in published patents.

### Example 3: Binding to Human Cancer Cell Line SHP-77 and Human Red Blood Cells

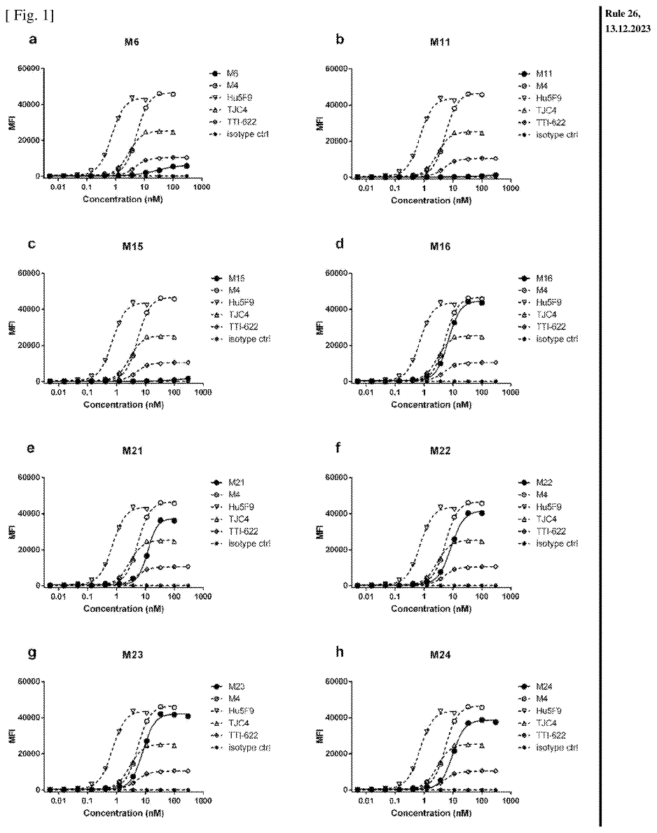

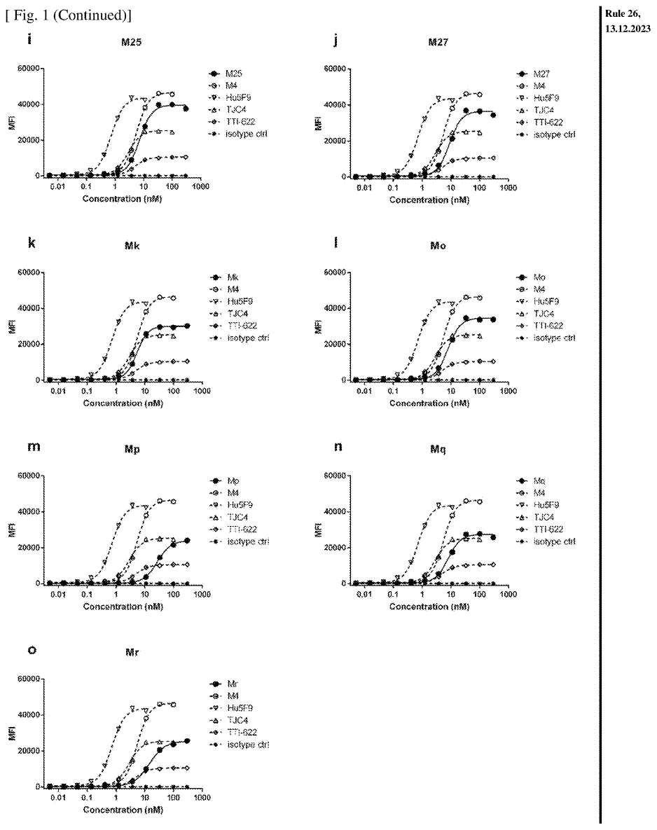

Briefly, SHP-77 tumor cells (ATCC, cat. no. CRL-2195) were cultured and plated in 96-well plates at 2×10⁵ cells/well and incubated with various concentrations of anti-CD47 antibodies, positive control molecules mentioned in EXAMPLE 2 and an isotype control IgG4,κ (Sino biological, cat. no. HG4K) at 37° C. for 30 minutes. Cells were washed three times followed by incubation with an Alexa Fluor 647-fluorescently-labeled goat anti-human IgG secondary antibody (Jackson ImmunoResearch Inc., cat. no. 109-605-098) at 37° C. for 30 minutes. Cells were washed three times and re-suspended in Dulbecco's Phosphate-Buffered Saline (DPBS) buffer. Samples were acquired on BD FACSCelesta flow cytometer (BD Biosciences) to determine the geometric mean fluorescent intensity (MFI) at each antibody concentration. Raw data were analyzed using FlowJo software. Half-maximal effective concentration (EC50) values were calculated using a sigmoidal dose-response curve in GraphPad Prism software (Table 3 and FIG. 1). Generally speaking, as the binding affinity of antibodies decreased, binding activity to SHP-77 cells decreases, as indicated by increasing EC50 values and decreasing maximal MFI values. Of all variants, M6, M11 and M15 binds tumor line with lowest binding activities, even lower than that of TTI-622.

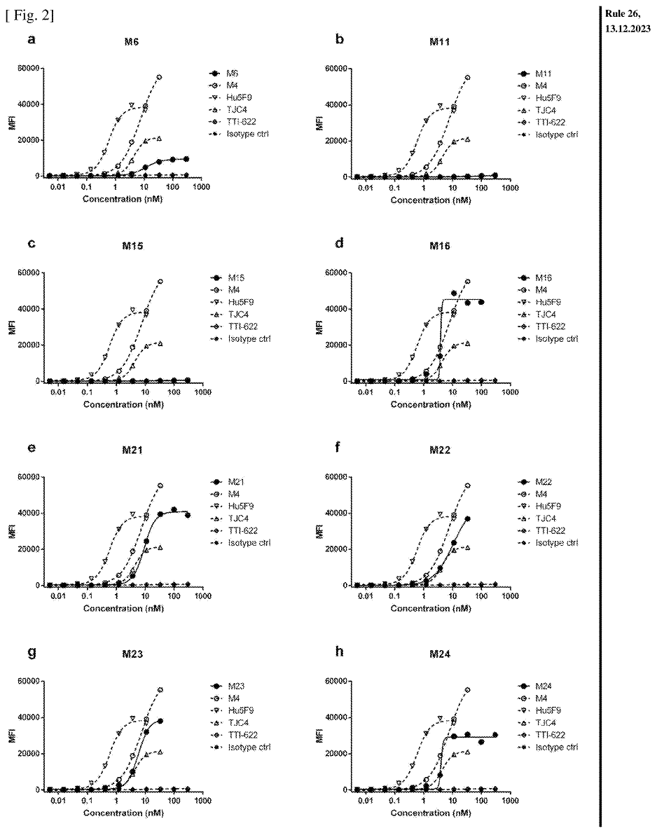

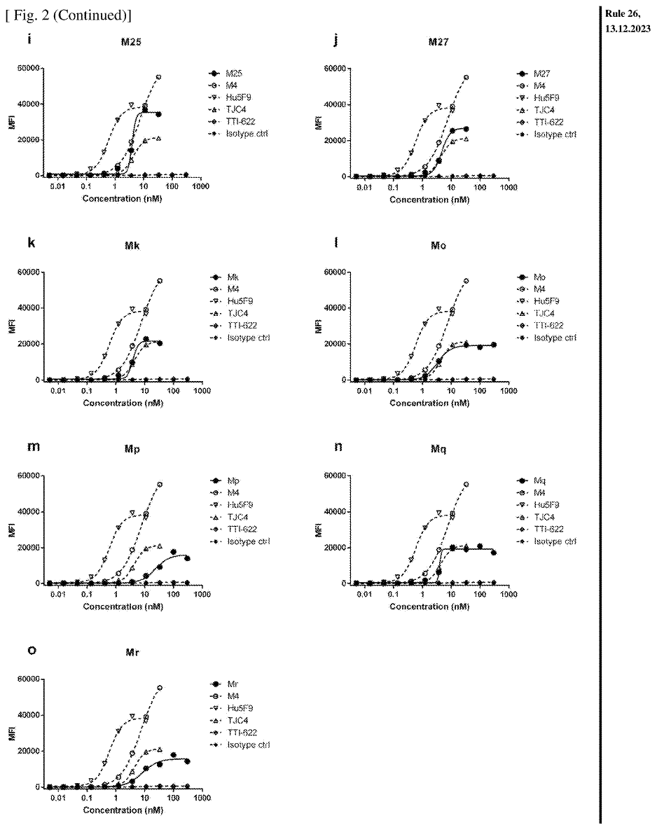

Similarly, binding to human red blood cells (RBCs) were also carried out using the same antibodies (Table 3 and FIG. 2). Similar trend was also observed for RBC binding, only the differences in EC50 and Maximal MFI values between antibodies were more pronounced.

TABLE 3
Binding activities of humanized antibodies.

| | | | SHP-77 | | RBC | |
|---|---|---|---|---|---|---|
| mAb | | | | Fitted | | Fitted |
| short | | | EC50 | Maximal | EC50 | maximal |
| name | VH | VL | (nM) | MFI | (nM) | MFI |
| M4 | VH4.M4 | VL1.M1 | 5.5 | 46571 | 7.6^{#} | 63756^{#} |
| M6 | VH5.M6 | VL1.M5 | 25.1 | 6252 | 11.1 | 9457 |
| M11 | VH5.M11 | VL1.M5 | 797.0 | 3995 | 177.8 | 1538 |
| M15 | VH5.M15 | VL1.M5 | 396.0 | 3682 | 155.3 | 926.5 |
| M16 | VH5.M16 | VL1.M5 | 6.9 | 44717 | 3.9* | 45333 |
| M21 | VH5.M21 | VL1.M5 | 11.9 | 37402 | 9.2 | 40835 |
| M22 | VH5.M22 | VL1.M5 | 8.4 | 41636 | 10.1^{#} | 44640^{#} |
| M23 | VH5.M23 | VL1.M5 | 8.1 | 42121 | 5.8 | 38844 |
| M24 | VH5.M24 | VL1.M5 | 9.8 | 38728 | 4.0 | 29250 |
| M25 | VH5.M25 | VL1.M5 | 7.1 | 39597 | 4.0 | 35466 |
| M27 | VH5.M27 | VL1.M5 | 9.0 | 36505 | 4.7 | 26932 |
| Mk | VH10.Mk | VL10.Me | 5.3 | 29953 | 3.9 | 21653 |
| Mo | VH10.Mo | VL10.Me | 7.9 | 34533 | 3.4 | 19168 |
| Mp | VH10.Mp | VL10.Me | 27.5 | 24090 | 23.6 | 16119 |
| Mq | VH10.Mq | VL10.Me | 8.0 | 27429 | 3.9 | 19375 |
| Mr | VH10.Mr | VL10.Me | 14.4 | 25385 | 8.1 | 15736 |

| | | | | |
|---|---|---|---|---|
| Hu5F9 | 0.7 | 43771 | 0.6 | 38436 |
| TJC4 | 2.8 | 25364 | 4.3 | 21427 |
| TTI-622 | 4.9 | 10455 | 9.7 | 643.8 |

*poor fitting, values are not accurate.
^{#}plateau not reached, values are not accurate.

### Example 4: Binding to Human and Cynomolgus CD47 Over-Expressing Cell Lines

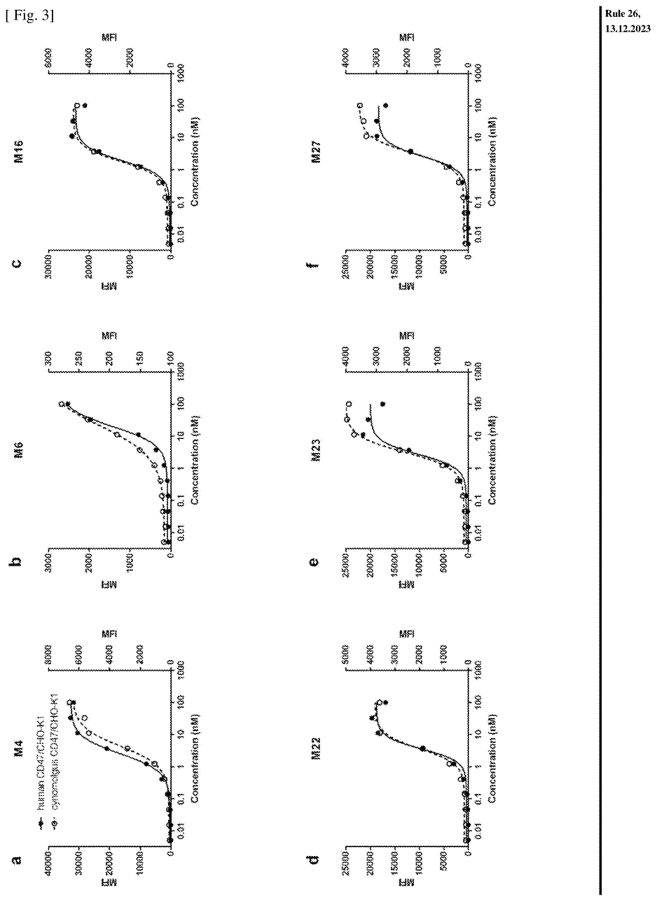

To determine the binding activity of anti-CD47 antibodies to human and cynomolgus CD47 expressing cells, CHO-K1 cells over-expressing human and cynomolgus CD47 were incubated with gradient concentrations of several selected antibodies produced in EXAMPLE 2, followed by incubation with an Alexa Fluor 647-fluorescently-labeled goat anti-human IgG secondary antibody (Jackson ImmunoResearch Inc., cat. no. 109-605-098) at 37° C. for 30 minutes. Samples were analyzed with flow cytometry. Binding activity of anti-CD47 antibodies were shown in FIG. 3 and Table 4. Most anti-CD47 antibody variants bound human and cynomolgus CD47 over-expressing cell lines with similar EC50 values (Table 4), however, the binding maximal signals of cynomolgus CD47 cells were much lower than those of human CD47 cells (FIG. 3), which probably could be due to the difference in CD47 expression levels on cells (human CD47-CHO-K1, 2.7 log shift compared to control vs. cynomolgus CD47-CHO-K1, 1.8 log shift compared to control).

TABLE 4
Binding activities of anti-CD47 antibodies to human
and cynomolgus CD47 CHO-K1 stable cell lines.

| mAb | EC50 (nM) | |
|---|---|---|
| short name | human CD47/CHO-K1 | cynomolgus CD47/CHO-K1 |
| M4 | 2.6 | 4.3 |
| M6 | 20.0^{#} | 22.5^{#} |
| M16 | 1.9 | 1.8 |
| M22 | 3.6 | 3.6 |
| M23 | 2.7 | 3.2 |
| M27 | 2.6 | 3.4 |
| Mk | 5.4^{#} | 6.4^{#} |
| Mq | 6.4^{#} | 8.4^{#} |
| Mr | 15.7^{#} | 49.9^{#} |
| Hu5F9 | 0.6 | 0.6 |
| TJC4 | 5.7^{#} | 9.7^{#} |
| TTI-622 | 10.4^{#} | 9.3^{#} |

^{#}plateau not reached, values are not accurate

### Example 5: In Vitro Functional Studies of Anti-CD47 Antibodies

Blocking the CD47-SIRPα interaction with anti-CD47 antibodies induces the phagocytosis of CD47-expressing tumor cells and RBCs by macrophages in vitro. Therefore, this assay was used for functional and safety studies. Monocytes were purified from peripheral blood mononuclear cells (PBMC, HemaCare, cat. no. PB009C-3) by negative selection using Pan Monocyte Isolation Kit (Miltenyi Biotech, cat. no. 130-096-537) according to the manufacturer's instructions. Monocyte-derived macrophages were induced by seeding cells at 1×10⁶ cells/mL in RPMI-1640 medium (Gibco, cat. no. 22400-089) supplemented with 10% heat-inactivated fetal calf serum (Gibco, cat. no. 10099-141C) and 100 ng/mL M-CSF (GenScript, cat. no. Z02924-50). Cells were cultured for 8 days to allow monocytes to differentiate into macrophages. CFSE-labeled CCRF-CEM tumor cells (ATCC, cat. no. CCL-119) were treated with various concentrations of either anti-CD47 antibodies or human IgG4 isotype control for 1 hour at 37° C., then co-cultured with violet-labeled macrophages at effector-to-target ratio of 1:1 for 1 hour at 37° C. After co-culture, cells were collected and analyzed by flow cytometry. Percentage of CFSE+ target cells was recorded. The phagocytosis of target cells induced by antibodies was calculated as follow:

$$tumor cell phagocytosis = \frac{\begin{matrix}percentage of target cell of ctrl - \\ percentage of target cell of sample\end{matrix}}{percentage of target cell of ctrl}$$

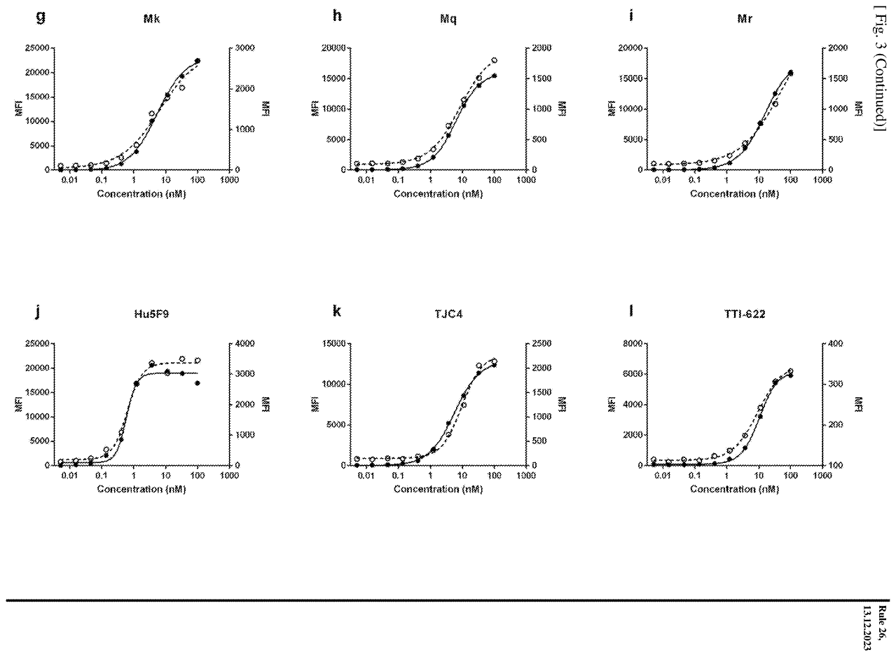

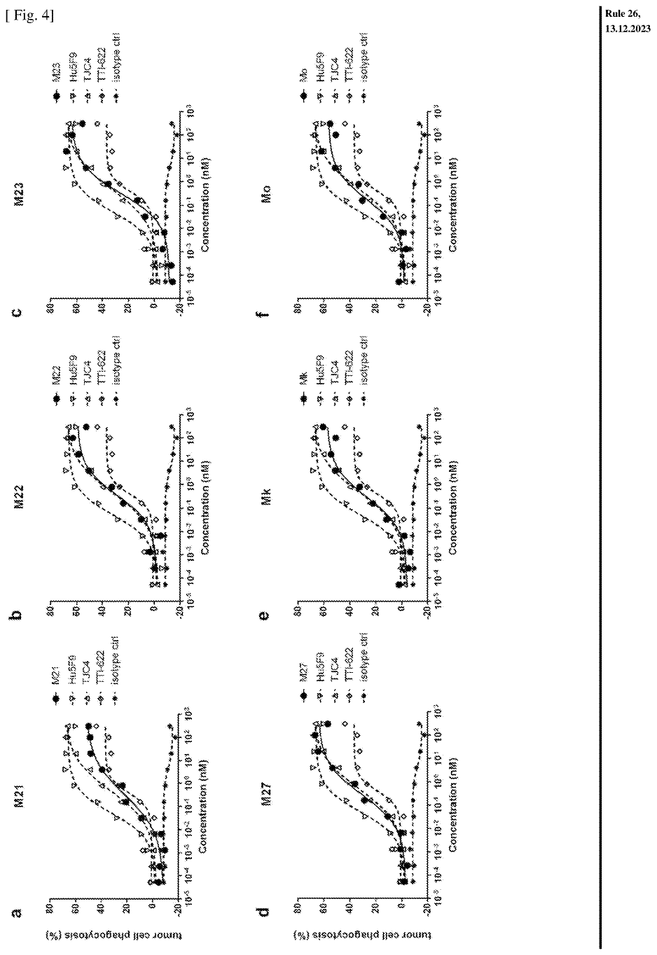

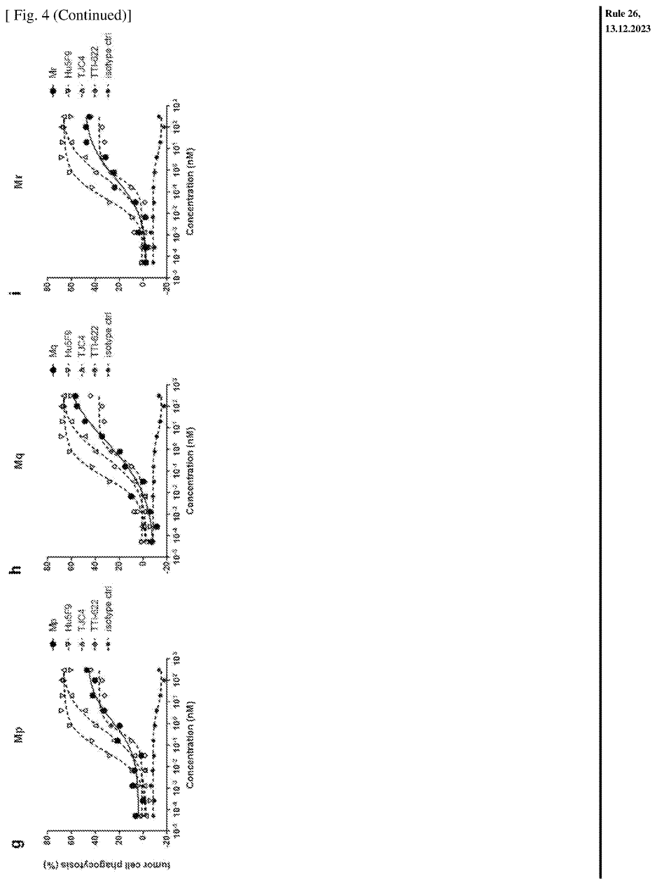

EC50 values were calculated using a sigmoidal dose-response curve in GraphPad Prism software (Table 5 and FIG. 4). The functional activities of M22, M23, M27, Mk and Mo are inferior to that of Hu5F9, similar to that of TJC4 and superior to that of TTI-622; whereas the functional activities of Mp, Mq and Mr are inferior to that of Hu5F9 and TJC4, similar to that of TTI-622. Generally speaking, the functional activities of anti-CD47 variants derived from 108C10A6 are consistent with the mono-valent binding affinity of variants, with only the exception of M21.

TABLE 5
Phagocytosis of tumor cells induced by anti-CD47 antibodies.

| | |
|---|---|
| mAb | CCRF-CEM phagocytosis |

| short name | EC50 (nM) | Phagocytosis (%) |
|---|---|---|
| M21 | 0.32 | 51.9 |
| M22 | 0.37 | 59.4 |
| M23 | 0.33 | 64.0 |
| M27 | 0.28 | 64.3 |
| Mk | 0.29 | 57.6 |
| Mo | 0.18 | 56.4 |
| Mp | 1.20 | 47.1 |
| Mq | 2.3^{#} | 71.7^{#} |
| Mr | 0.43 | 49.8 |
| Hu5F9 | 0.05 | 66.1 |
| TJC4 | 0.49 | 68.6 |
| TTI-622 | 0.40 | 36.6 |

^{#}plateau not reached, values are not accurate

### Example 6: Phagocytosis of RBCs Induced by CD47 Targeting Molecules

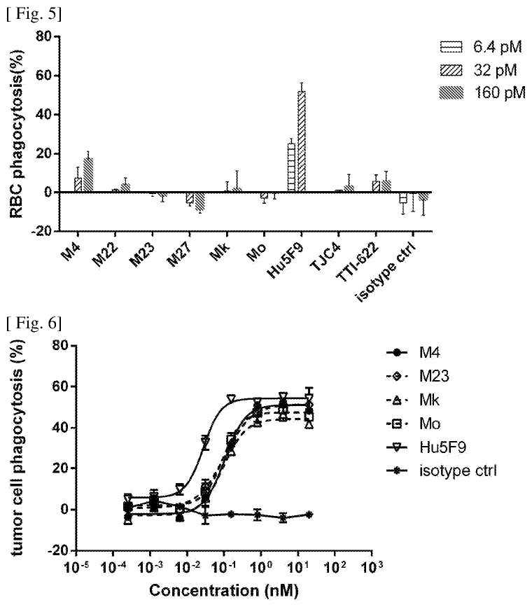

Phagocytosis of RBCs induced by antibodies at fixed concentrations (6.4 pM, 32 pM and 160 pM) was carried out at effector-to-target ratio of 1:1. As can be seen from FIG. 5, Hu5F9 induced the most phagocytosis of RBCs and at lower antibody concentrations (6.4 pM and 32 pM). The original humanized 108C10A6 antibody (i.e. M4) with high CD47-binding affinity also showed 20% of phagocytosis of RBCs at 160 pM, however humanized antibody variants with reduced affinities, i.e. M22, M23, M27, Mo and Mk and benchmark molecules such as TJC4 and TTI-622 showed little to no phagocytosis (FIG. 5), suggesting these antibodies may have a better safety profile as far as anemia is concerned.

### Example 7: Comparison of Variants of Humanized 108C10A6 Antibody in Efficacy and Toxicity

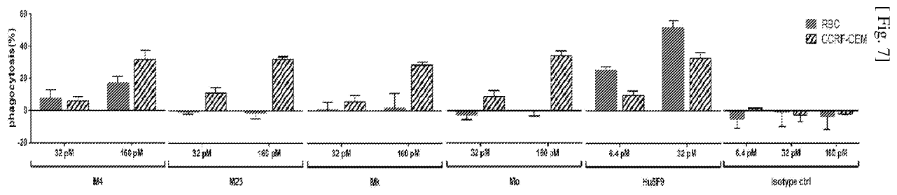

To determine which variant(s) of humanized 108C10A6 antibody may have the greatest therapeutic window in clinical practice, CCRF-CEM cancer cell phagocytosis was carried out as described in EXAMPLE 5 using M4, M23, Mk, Mo together with positive and negative control antibodies. As can be seen from FIG. 6, all 4 selected humanized variants showed comparable tumor cell efficacy and potency. The phagocytosis of tumor cells at fixed concentrations (Hu5F9 at 6.4 pM and 32 pM, and the other antibodies at 32 pM and 160 pM) was plotted together with the phagocytosis of RBCs shown in EXAMPLE 6. For Hu5F9, the phagocytosis of RBCs was more significant than that of tumor cells. At the indicated concentrations, M4, M23, Mk and Mo showed similar tumor cell phagocytosis; on the other hand, M23, Mk and Mo showed little to no RBC phagocytosis, whereas M4 showed obvious RBC phagocytosis (FIG. 7). The result suggests that M23, Mk and Mo have better efficacy to toxicity ratio than M4 and Hu5F9.

### Example 8: RBC Hemagglutination Induced by Anti-CD47 Antibodies

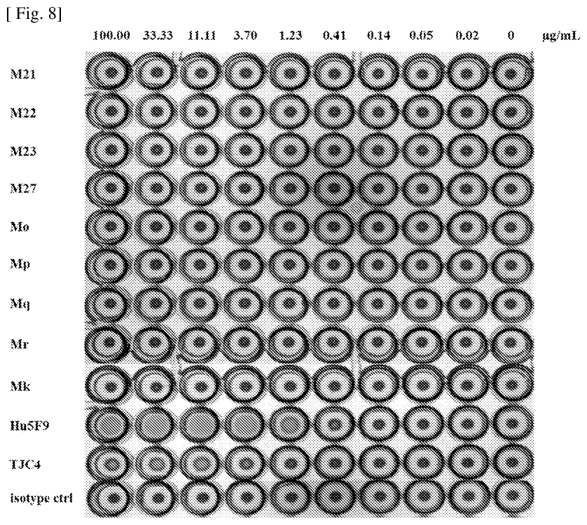

Some validated CD47-blocking antibody under clinical investigation, such as Hu5F9, induces hemagglutination and anemia (Advani R. et al. 2018). To overcome this limitation, therapeutic agents with limited hemagglutination are needed. RBC hemagglutination induced by anti-CD47 antibodies was carried out on selected variants of humanized anti-CD47 antibodies. Human RBCs were washed three times with DPBS. Increasing concentrations of anti-CD47 antibodies (up to 100 μg/mL) were added to wells containing RBCs and the plates were incubated for 2 hours at 37° C. Imaging of the plates was carried out afterwards. A diffuse hazy pattern was observed for Hu5F9 at concentration of 0.41 pg/mL and above and for TJC4 at concentration of 3.70 pg/mL and above, indicating hemagglutination; whereas small punctate circles were observed for the other anti-CD47 antibodies at all concentrations, indicating no obvious hemagglutination (FIG. 8).

### Example 9: In Vivo Efficacy of Humanized Anti-CD47 Antibodies

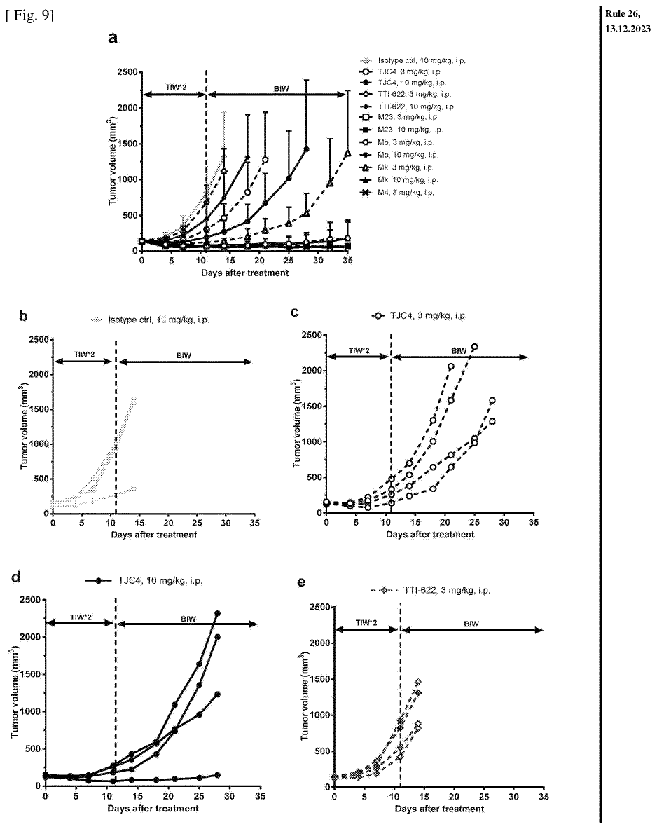

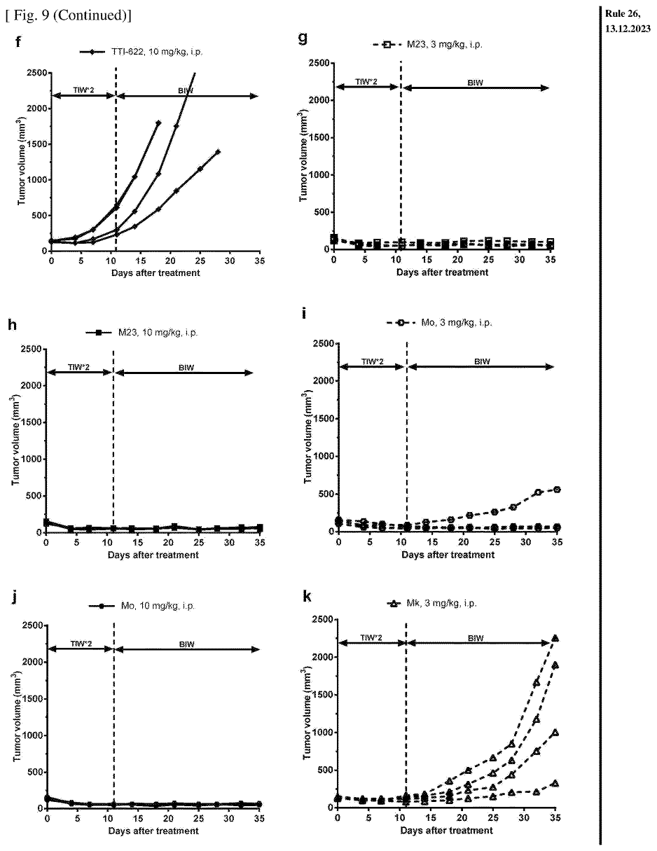

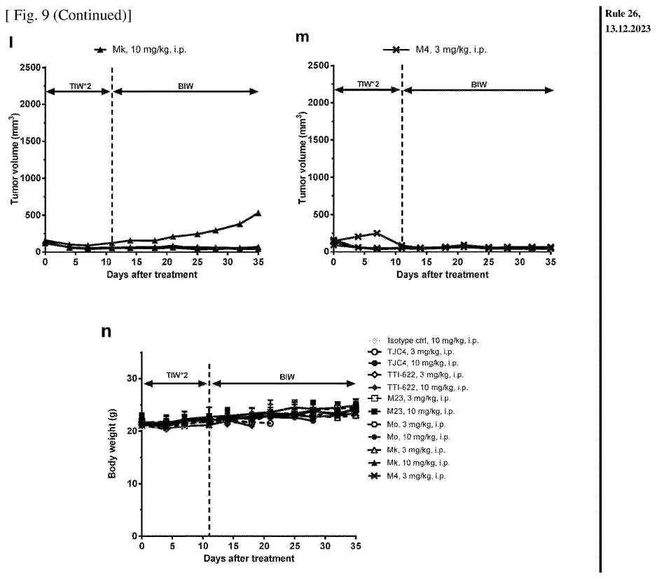

The in vivo efficacy of anti-CD47 antibodies was studied using a tumor cell derived xenograft model. Briefly, SHP-77 tumor cells were cultured, and 1×10⁷ cells were injected subcutaneously at the flank of female NCG (NOD/ShiLtJGpt-PrkdC^{em26Cd52}Il2rg^{em26Cd22}/Gpt, GemPharmatech) mice (6-7 weeks of age). The sizes of tumors were measured using a caliper and tumor volumes were calculated as Length×Width×Width/2. When tumors were palpable and the average volume reached ˜140 mm³, mice were randomized into groups of 4 mice and were treated with anti-CD47 antibodies. CD47-targeting reagents were dosed at 3 mg/kg and 10 mg/kg intraperitoneally 3 times a week for the first 5 doses and twice a week for the next 7 doses. Body weights were measured throughout the study. The animals were sacrificed when the tumor volume reached 2000 mm³ or when the study ended (35 days post-treatment). As shown in FIG. 9, benchmark molecules such as TJC4 and TTI-622 showed very little tumor growth inhibition activity, whereas variants of humanized 108C10A6 antibodies showed much better tumor growth inhibition. Of all variants, M4 and M23 showed complete tumor growth inhibition even at low dose of 3 mg/kg. In 1 out of 4 mice of 3 mg/kg Mo group, the tumor relapsed, suggesting that the in vivo efficacy of Mo may be slightly inferior to M4 and M23. Mk showed the least tumor growth inhibition out of the 4 humanized variants. There was no sign of significant loss of body weight throughout the study (FIG. 9n).

### REFERENCES

Brown E J, Frazier W A. Integrin-associated protein (CD47) and its ligands. Trends Cell Biol., 11 (2001), pp. 130-135.
Brown E J. Integrin-associated protein (CD47): an unusual activator of G protein signaling. J. Clin. Invest., 107 (2001), pp. 1499-1500.
Isenberg J S, Annis D S, Pendrak M L, Ptaszynska M, Frazier W A, Mosher D F, Roberts D D. Differential interactions of thrombospondin-1, -2, and -4 with CD47 and effects on cGMP signaling and ischemic injury responses. J Biol Chem. 2009; 284:1116-1125.
Advani R, Flinn I, Popplewell L, Forero A, Bartlett N L, Ghosh N, et al. CD47 blockade by Hu5F9-G4 and rituximab in non-Hodgkin's lymphoma. N Engl J Med. 2018; 379(18):1711-21.
Winter G, Harris W J. Humanized antibodies. Trends Pharmacol Sci 1993; 14:139-43.
Robert A. Rebres, Louise E. Vaz, Jennifer M. Green, and Eric J. Brown Normal ligand binding and signaling by CD47 (integrin-associated protein) requires a long range disulfide bond between the extracellular and membrane-spanning domains. J Biol Chem. 2001 Sep. 14; 276(37):34607-16.
A. Wörn, A. Plückthun Different equilibrium stability behavior of ScFv fragments: identification, classification, and improvement by protein engineering. Biochemistry, 38 (1999), pp. 8739-8750.

TABLE 6
SEQUENCES

| SEQ ID | | |
|---|---|---|
| NO: | Description | SEQUENCE |
| 1 | Human CD47-C33G | QLLENKTKSVEFTFGNDTVVIPCFVTNMEAQNTTEVYVKWKEKGRD |
| | | IYTFDGALNKSTVPTDESSAKIEVSQLLKGDASLKMDKSDAVSHTG |
| | | NYTCEVTELTREGETIIELKYRVVS |
| 2 | 108VH | QMQLQQSGPQLVRPGASVKISCKTSGYSFTHHWIHWMKQRPGQGLE |
| | | WIGMIDPSDSETRLSQKFKDKATLTVDASSSTAYMQLNSPTSEDSA |
| | | LYFCARLGRYYFDYWGQGTTLTVSS |
| 3 | 108VL | NIVMTQSPRSMSMSVGERVTLSCKASENVGTYISWYQQKPDQSPKL |
| | | LIYGASNRYTGVPDRFTGSGSGTDFTLTISTVQAEDLADYHCGESY |
| | | GHLYTFGGGTKLEIK |
| 4 | 108VH4.M4 | EVQLVQSGAEVKKPGSSVKVSCKASGYSFTHHWIHWVRQAPGQGLE |
| | | WMGMIDASDSETRLSQKFKDRVTITADKSTSTAYMELSSLRSEDTA |
| | | VYYCARLGRYYFDYWGQGTTVTVSS |
| 5 | 108VL1.M1 | EIVLTQSPATLSLSPGERATLSCRASENVGTYISWYQQKPGQAPRL |
| | | LIYGASNRYTGIPARFSGSGSGTDFTLTISSLEPEDFAVYYCGESY |
| | | GHLYTFGGGTKVEIK |
| 6 | 108VH5 | EVQLVQSGAEVKKPGASVKVSCKASGYSFTHHWIHWVRQAPGQGLE |
| | | WMGMIDPSDSETRLSQKFKDRVTMTRDTSTSTVYMELSSLRSEDTA |
| | | VYYCARLGRYYFDYWGQGTLVTVSS |
| 7 | 108VL1.M5 | EIVLTQSPATLSLSPGERATLSCRASQSVSSYLAWYQQKPGQAPRL |
| | | LIYGASNRATGIPARFSGSGSGTDFTLTISSLEPEDFAVYYCGESY |
| | | GHLYTFGGGTKVEIK |
| 8 | 108VH5.M5 | EVQLVQSGAEVKKPGASVKVSCKASGYTFTHHWIHWVRQAPGQGLE |
| | | WMGMIDASDSETRLSQKFKDRVTMTRDTSTSTVYMELSSLRSEDTA |
| | | VYYCARLGRYYFDYWGQGTLVTVSS |
| 9 | 108VH5.M6 | EVQLVQSGAEVKKPGASVKVSCKASGYTFTHHYMHWVRQAPGQGLE |
| | | WMGMIDASDSETRLSQKFKDRVTMTRDTSTSTVYMELSSLRSEDTA |
| | | VYYCARLGRYYFDYWGQGTLVTVSS |
| 10 | 108VH5.M8 | EVQLVQSGAEVKKPGASVKVSCKASGYSFTHHWIHWVRQAPGQGLE |
| | | WMGMIDASDSETRYAQKFKDRVTMTRDTSTSTVYMELSSLRSEDTA |
| | | VYYCARLGRYYFDYWGQGTLVTVSS |
| 11 | 108VH5.M9 | EVQLVQSGAEVKKPGASVKVSCKASGYSFTHHWIHWVRQAPGQGLE |
| | | WMGMIDASDSETRLSQKFQGRVTMTRDTSTSTVYMELSSLRSEDTA |
| | | VYYCARLGRYYFDYWGQGTLVTVSS |
| 12 | 108VH5.M10 | EVQLVQSGAEVKKPGASVKVSCKASGYTFTHHWIHWVRQAPGQGLE |
| | | WMGMIDASDSETRYAQKFQGRVTMTRDTSTSTVYMELSSLRSEDTA |
| | | VYYCARLGRYYFDYWGQGTLVTVSS |
| 13 | 108VH5.M11 | EVQLVQSGAEVKKPGASVKVSCKASGYTFTHHYIHWVRQAPGQGLE |
| | | WMGMIDASDSETRYAQKFQGRVTMTRDTSTSTVYMELSSLRSEDTA |
| | | VYYCARLGRYYFDYWGQGTLVTVSS |
| 14 | 108VH5.M12 | EVQLVQSGAEVKKPGASVKVSCKASGYTFTHHWMHWVRQAPGQGLE |
| | | WMGMIDASDSETRYAQKFQGRVTMTRDTSTSTVYMELSSLRSEDTA |
| | | VYYCARLGRYYFDYWGQGTLVTVSS |
| 15 | 108VH5.M14 | EVQLVQSGAEVKKPGASVKVSCKASGYTFTHHWIHWVRQAPGQGLE |
| | | WMGMIDASDGETRYAQKFQGRVTMTRDTSTSTVYMELSSLRSEDTA |
| | | VYYCARLGRYYFDYWGQGTLVTVSS |
| 16 | 108VH5.M15 | EVQLVQSGAEVKKPGASVKVSCKASGYTFTHHWIHWVRQAPGQGLE |
| | | WMGMIDASDSSTRYAQKFQGRVTMTRDTSTSTVYMELSSLRSEDTA |
| | | VYYCARLGRYYFDYWGQGTLVTVSS |
| 17 | 108VH5.M16 | EVQLVQSGAEVKKPGASVKVSCKASGYTFTHHWMHWVRQAPGQGLE |
| | | WMGMIDASDGETRYAQKFQGRVTMTRDTSTSTVYMELSSLRSEDTA |
| | | VYYCARLGRYYFDYWGQGTLVTVSS |
| 18 | 108VH5.M17 | EVQLVQSGAEVKKPGASVKVSCKASGYTFTHHWMHWVRQAPGQGLE |
| | | WMGMIDASEGETRYAQKFQGRVTMTRDTSTSTVYMELSSLRSEDTA |
| | | VYYCARLGRYYFDYWGQGTLVTVSS |
| 19 | 108VH5.M18 | EVQLVQSGAEVKKPGASVKVSCKASGYTFTHHWMHWVRQAPGQGLE |
| | | WMGMIDASESETRYAQKFQGRVTMTRDTSTSTVYMELSSLRSEDTA |
| | | VYYCARLGRYYFDYWGQGTLVTVSS |
| 20 | 108VH5.M20 | EVQLVQSGAEVKKPGASVKVSCKASGYTFTHHWMHWVRQAPGQGLE |
| | | WMGMIDASDAETRYAQKFQGRVTMTRDTSTSTVYMELSSLRSEDTA |
| | | VYYCARLGRYYFDYWGQGTLVTVSS |
| 21 | 108VH5.M21 | EVQLVQSGAEVKKPGASVKVSCKASGYTFTHHWMHWVRQAPGQGLE |
| | | WMGMIDASDFETRYAQKFQGRVTMTRDTSTSTVYMELSSLRSEDTA |
| | | VYYCARLGRYYFDYWGQGTLVTVSS |
| 22 | 108VH5.M22 | EVQLVQSGAEVKKPGASVKVSCKASGYTFTHHWMHWVRQAPGQGLE |
| | | WMGMIDASDIETRYAQKFQGRVTMTRDTSTSTVYMELSSLRSEDTA |
| | | VYYCARLGRYYFDYWGQGTLVTVSS |
| 23 | 108VH5.M23 | EVQLVQSGAEVKKPGASVKVSCKASGYTFTHHWMHWVRQAPGQGLE |
| | | WMGMIDASDKETRYAQKFQGRVTMTRDTSTSTVYMELSSLRSEDTA |
| | | VYYCARLGRYYFDYWGQGTLVTVSS |
| 24 | 108VH5.M24 | EVQLVQSGAEVKKPGASVKVSCKASGYTFTHHWMHWVRQAPGQGLE |
| | | WMGMIDASDLETRYAQKFQGRVTMTRDTSTSTVYMELSSLRSEDTA |
| | | VYYCARLGRYYFDYWGQGTLVTVSS |
| 25 | 108VH5.M25 | EVQLVQSGAEVKKPGASVKVSCKASGYTFTHHWMHWVRQAPGQGLE |
| | | WMGMIDASDRETRYAQKFQGRVTMTRDTSTSTVYMELSSLRSEDTA |
| | | VYYCARLGRYYFDYWGQGTLVTVSS |
| 26 | 108VH5.M26 | EVQLVQSGAEVKKPGASVKVSCKASGYTFTHHWMHWVRQAPGQGLE |
| | | WMGMIDASDVETRYAQKFQGRVTMTRDTSTSTVYMELSSLRSEDTA |
| | | VYYCARLGRYYFDYWGQGTLVTVSS |
| 27 | 108VH5.M27 | EVQLVQSGAEVKKPGASVKVSCKASGYTFTHHWMHWVRQAPGQGLE |
| | | WMGMIDASDYETRYAQKFQGRVTMTRDTSTSTVYMELSSLRSEDTA |
| | | VYYCARLGRYYFDYWGQGTLVTVSS |
| 28 | IgG4 heavy chain | ASTKGPSVFPLAPCSRSTSESTAALGCLVKDYFPEPVTVSWNSGAL |
| | constant region with | TSGVHTFPAVLQSSGLYSLSSVVTVPSSSLGTKTYTCNVDHKPSNT |
| | S228P mutation | KVDKRVESKYGPPCPPCPAPEFLGGPSVFLFPPKPKDTLMISRTPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLGK |
| 29 | kappa light chain | RTVAAPSVFIFPPSDEQLKSGTASVVCLLNNFYPREAKVQWKVDNA |
| | constant region | LQSGNSQESVTEQDSKDSTYSLSSTLTLSKADYEKHKVYACEVTHQ |
| | | GLSSPVTKSENRGEC |
| 30 | 108VL10.M1 | DIQMTQSPSSLSASVGDRVTITCRASENVGTYISWYQQKPGKAPKL |
| | | LIYGASNRYTGVPSRESGSRSGTDFTLTISSLQPEDFATYYCGESY |
| | | GHLYTFGQGTKVEIK |
| 31 | 108VH10 | EVQLVESGGGLVQPGGSLRLSCAASGYSFTHHWIHWVRQAPGKGLE |
| | | WVAMIDPSDSETRLSQKFKDRFTISADTSKNTAYLQMNSLRAEDTA |
| | | VYYCSRLGRYYFDYWGQGTLVTVSS |
| 32 | 108VL10.Ma | DIQMTQSPSSLSASVGDRVTITCRASQSISSYLNWYQQKPGKAPKL |
| | | LIYGASNRYTGVPSRFSGSRSGTDFTLTISSLQPEDFATYYCGESY |
| | | GHLYTFGQGTKVEIK |
| 33 | 108VL10.Mb | DIQMTQSPSSLSASVGDRVTITCRASENVGTYISWYQQKPGKAPKL |
| | | LIYAASNRYTGVPSRFSGSRSGTDFTLTISSLQPEDFATYYCGESY |
| | | GHLYTFGQGTKVEIK |
| 34 | 108VL10.Mc | DIQMTQSPSSLSASVGDRVTITCRASENVGTYISWYQQKPGKAPKL |
| | | LIYGASSRYTGVPSRFSGSRSGTDFTLTISSLQPEDFATYYCGESY |
| | | GHLYTFGQGTKVEIK |
| 35 | 108VL10.Md | DIQMTQSPSSLSASVGDRVTITCRASENVGTYISWYQQKPGKAPKL |
| | | LIYGASNRYSGVPSRESGSRSGTDFTLTISSLQPEDFATYYCGESY |
| | | GHLYTFGQGTKVEIK |
| 36 | 108VL10.Me | DIQMTQSPSSLSASVGDRVTITCRASQSISSYLNWYQQKPGKAPKL |
| | | LIYGASSRYSGVPSRFSGSRSGTDFTLTISSLQPEDFATYYCGESY |
| | | GHLYTFGQGTKVEIK |
| 37 | 108VH10.Ma | EVQLVESGGGLVQPGGSLRLSCAASGFTFSHHWIHWVRQAPGKGLE |
| | | WVAMIDPSDSETRLSQKFKDRFTISADTSKNTAYLQMNSLRAEDTA |
| | | VYYCSRLGRYYFDYWGQGTLVTVSS |
| 38 | 108VH10.Mb | EVQLVESGGGLVQPGGSLRLSCAASGYSFTHHWMHWVRQAPGKGLE |
| | | WVAMIDPSDSETRLSQKFKDRFTISADTSKNTAYLQMNSLRAEDTA |
| | | VYYCSRLGRYYFDYWGQGTLVTVSS |
| 39 | 108VH10.Mc | EVQLVESGGGLVQPGGSLRLSCAASGYSFTHHWISWVRQAPGKGLE |
| | | WVAMIDPSDSETRLSQKFKDRFTISADTSKNTAYLQMNSLRAEDTA |
| | | VYYCSRLGRYYFDYWGQGTLVTVSS |
| 40 | 108VH10.Md | EVQLVESGGGLVQPGGSLRLSCAASGYSFTHHWIHWVRQAPGKGLE |
| | | WVAMIKPSDSETRLSQKFKDRFTISADTSKNTAYLQMNSLRAEDTA |
| | | VYYCSRLGRYYFDYWGQGTLVTVSS |
| 41 | 108VH10.Me | EVQLVESGGGLVQPGGSLRLSCAASGYSFTHHWIHWVRQAPGKGLE |
| | | WVAMIDQSDSETRLSQKFKDRETISADTSKNTAYLQMNSLRAEDTA |
| | | VYYCSRLGRYYFDYWGQGTLVTVSS |
| 42 | 108VH10.Mf | EVQLVESGGGLVQPGGSLRLSCAASGYSFTHHWIHWVRQAPGKGLE |
| | | WVAMIDPSDSETRLVDSVKGRFTISADTSKNTAYLQMNSLRAEDTA |
| | | VYYCSRLGRYYFDYWGQGTLVTVSS |
| 43 | 108VH10.Mg | EVQLVESGGGLVQPGGSLRLSCAASGFTFSHHWIHWVRQAPGKGLE |
| | | WVAMIDASDSETRLSQKFKDRFTISADTSKNTAYLQMNSLRAEDTA |
| | | VYYCSRLGRYYFDYWGQGTLVTVSS |
| 44 | 108VH10.Mh | EVQLVESGGGLVQPGGSLRLSCAASGFTFSHHWIHWVRQAPGKGLE |
| | | WVAMIDASDSETRLVQKFKDRFTISADTSKNTAYLQMNSLRAEDTA |
| | | VYYCSRLGRYYFDYWGQGTLVTVSS |
| 45 | 108VH10.Mi | EVQLVESGGGLVQPGGSLRLSCAASGFTFSHHWIHWVRQAPGKGLE |
| | | WVAMIDASDSETRLSDKFKDRFTISADTSKNTAYLQMNSLRAEDTA |
| | | VYYCSRLGRYYFDYWGQGTLVTVSS |
| 46 | 108VH10.Mj | EVQLVESGGGLVQPGGSLRLSCAASGFTFSHHWIHWVRQAPGKGLE |
| | | WVAMIDASDSETRLSQSFKDRFTISADTSKNTAYLQMNSLRAEDTA |
| | | VYYCSRLGRYYFDYWGQGTLVTVSS |
| 47 | 108VH10.Mk | EVQLVESGGGLVQPGGSLRLSCAASGFTFSHHWIHWVRQAPGKGLE |
| | | WVAMIDASDSETRLSQKVKDRFTISADTSKNTAYLQMNSLRAEDTA |
| | | VYYCSRLGRYYFDYWGQGTLVTVSS |
| 48 | 108VH10.Ml | EVQLVESGGGLVQPGGSLRLSCAASGFTFSHHWIHWVRQAPGKGLE |
| | | WVAMIDASDSETRLSQKFKGRFTISADTSKNTAYLQMNSLRAEDTA |
| | | VYYCSRLGRYYFDYWGQGTLVTVSS |
| 49 | 108VH10.Mo | EVQLVESGGGLVQPGGSLRLSCAASGFTFSHHWIHWVRQAPGKGLE |
| | | WVAMIDASDSETRLSDSFKGRFTISADTSKNTAYLQMNSLRAEDTA |
| | | VYYCSRLGRYYFDYWGQGTLVTVSS |
| 50 | 108VH10.Mp | EVQLVESGGGLVQPGGSLRLSCAASGFTFSHHWIHWVRQAPGKGLE |
| | | WVAMIDASDSETRLVDSFKGRFTISADTSKNTAYLQMNSLRAEDTA |
| | | VYYCSRLGRYYFDYWGQGTLVTVSS |
| 51 | 108VH10.Mq | EVQLVESGGGLVQPGGSLRLSCAASGFTFSHHWIHWVRQAPGKGLE |
| | | WVAMIDASDSETRLSDSVKGRFTISADTSKNTAYLQMNSLRAEDTA |
| | | VYYCSRLGRYYFDYWGQGTLVTVSS |
| 52 | 108VH10.Mr | EVQLVESGGGLVQPGGSLRLSCAASGFTFSHHWIHWVRQAPGKGLE |
| | | WVAMIDASDSETRLVDSVKGRFTISADTSKNTAYLQMNSLRAEDTA |
| | | VYYCSRLGRYYFDYWGQGTLVTVSS |
| 53 | HCDR1 | GYSFTHHWIH |
| | 108VH/108VH4.M4/ | |
| | 108VH5/108VH5.M8/ | |
| | 108VH5.M9/108VH10/ | |
| | 108VH10.Md/108VH10.Me/ | |
| | 108VH10.Mf | |
| 54 | HCDR1 | GYTFTHHWIH |
| | 108VH5.M5/108VH5.M10/ | |
| | 108VH5.M14/108VH5.M15 | |
| 55 | HCDR1 | GYTFTHHYMH |
| | 108VH5.M6 | |
| 56 | HCDRI | GYTFTHHYIH |
| | 108VH5.M11 | |
| 57 | HCDR1 | GYTFTHHWMH |
| | 108VH5.M12/M16/M17/ | |
| | M18/M20/M21/M22/ | |
| | M23/M24/M25/M26/M27 | |
| 58 | HCDR1 | GFTFSHHWIH |
| | 108VH10.Ma/Mg/Mh/ | |
| | Mi/Mj/Mk/Ml/Mo/Mp/Mq/ | |
| | Mr | |
| 59 | HCDR1 | GYSFTHHWMH |
| | 108VH10.Mb | |
| 50 | HCDR1 | GYSFTHHWIS |
| | 108VH10.Mc | |
| 61 | HCDR2 | MIDPSDSETRLSQKFKD |
| | 108VH/108VH5/ | |
| | 108VH10/108VH10.Ma/ | |
| | Mb/Mc | |
| 62 | HCDR2 | MIDASDSETRLSQKFKD |
| | 108VH4.M4/108VH5.M5/ | |
| | 108VH5.M6/108VH10.Mg | |
| 63 | HCDR2 | MIDASDSETRYAQKEKD |
| | 108VH5.M8 | |
| 64 | HCDR2 | MIDASDSETRLSQKFQG |
| | 108VH5.M9 | |
| 65 | HCDR2 | MIDASDSETRYAQKFQG |
| | 108VH5.M10/M11/M12 | |
| 66 | HCDR2 | MIDASDGETRYAQKFQG |
| | 108VH5.M14/M16 | |
| 67 | HCDR2 | MIDASDSSTRYAQKFQG |
| | 108VH5.M15 | |
| 68 | HCDR2 | MIDASEGETRYAQKFQG |
| | 108VH5.M17 | |
| 69 | HCDR2 | MIDASESETRYAQKFQG |
| | 108VH5.M18 | |
| 70 | HCDR2 | MIDASDAETRYAQKFQG |
| | 108VH5.M20 | |
| 71 | HCDR2 | MIDASDFETRYAQKFQG |
| | 108VH5.M21 | |
| 72 | HCDR2 | MIDASDIETRYAQKFQG |
| | 108VH5.M22 | |
| 73 | HCDR2 | MIDASDKETRYAQKFQG |
| | 108VH5.M23 | |
| 74 | HCDR2 | MIDASDLETRYAQKFQG |
| | 108VH5.M24 | |
| 75 | HCDR2 | MIDASDRETRYAQKFQG |
| | 108VH5.M25 | |
| 76 | HCDR2 | MIDASDVETRYAQKFQG |
| | 108VH5.M26 | |
| 77 | HCDR2 | MIDASDYETRYAQKFQG |
| | 108VH5.M27 | |
| 78 | HCDR2 | MIKPSDSETRLSQKFKD |
| | 108VH10.Md | |
| 79 | HCDR2 | MIDQSDSETRLSQKFKD |
| | 108VH10.Me | |
| 80 | HCDR2 | MIDPSDSETRLVDSVKG |
| | 108VH10.Mf | |
| 81 | HCDR2 | MIDASDSETRLVQKFKD |
| | 108VH10.Mh | |
| 82 | HCDR2 | MIDASDSETRLSDKFKD |
| | 108VH10.Mi | |
| 83 | HCDR2 | MIDASDSETRLSQSEKD |
| | 108VH10.Mj | |
| 84 | HCDR2 | MIDASDSETRLSQKVKD |
| | 108VH10.Mk | |
| 85 | HCDR2 | MIDASDSETRLSQKFKG |
| | 108VH10.Ml | |
| 86 | HCDR2 | MIDASDSETRLSDSFKG |
| | 108VH10.Mo | |
| 87 | HCDR2 | MIDASDSETRLVDSFKG |
| | 108VH10.Mp | |
| 88 | HCDR2 | MIDASDSETRLSDSVKG |
| | 108VH10.Mq | |
| 89 | HCDR2 | MIDASDSETRLVDSVKG |
| | 108VH10.Mr | |
| 90 | HCDR3 | LGRYYFDY |
| | 108VH/all variants | |
| 91 | LCDR1 | KASENVGTYIS |
| | 108VL | |
| 92 | LCDR1 | RASENVGTYIS |
| | 108VL1.M1 | |
| 93 | LCDR1 | RASQSVSSYLA |
| | 108VL1.M5 | |
| 94 | LCDR1 | RASENVGTYIS |
| | 108VL10.M1 | |
| 95 | LCDR1 | RASQSISSYLN |
| | 108VL 10.Me | |
| 96 | LCDR2 | GASNRYT |
| | 108VL1/108VL1.M1 | |
| 97 | LCDR2 | GASNRAT |
| | 108VL1.M5 | |
| 98 | LCDR2 | GASSRYS |
| | 108VL 10.Me | |
| 99 | LCDR3 | GESYGHLYT |
| | 108VL/all variants | |
| 100 | HFR1 | EVQLVQSGAEVKKPGASVKVSCKAS |
| | VH5-series variants | |
| 101 | HFR2 | WVRQAPGQGLEWMG |
| | VH5-series variants | |
| 102 | HFR3 | RVTMTRDTSTSTVYMELSSLRSEDTAVYYCAR |
| | VH5-series variants | |
| 103 | HFR4 | WGQGTLVTVSS |
| | VH5-series variants | |
| 104 | HFR1 | EVQLVESGGGLVQPGGSLRLSCAAS |
| | VH10-series variants | |
| 105 | HFR2 | WVRQAPGKGLEWVA |
| | VH10-series variants | |
| 106 | HFR3 | RFTISADTSKNTAYLQMNSLRAEDTAVYYCSR |
| | VH10-series variants | |
| 107 | HFR4 | WGQGTLVTVSS |
| | VH10-series variants | |
| 108 | LFR1 | EIVLTQSPATLSLSPGERATLSC |
| | 108VL1.M5 | |
| 109 | LFR2 | WYQQKPGQAPRLLIY |
| | 108VL1.M5 | |
| 110 | LFR3 | GIPARFSGSGSGTDFTLTISSLEPEDFAVYYC |
| | 108VL1.M5 | |
| 111 | LFR4 | FGGGTKVEIK |
| | 108VL1.M5 | |
| 112 | LFR1 | DIQMTQSPSSLSASVGDRVTITC |
| | 108VL10.Me | |
| 113 | LFR2 | WYQQKPGKAPKLLIY |
| | 108VL10.Me | |
| 114 | LFR3 | GVPSRESGSRSGTDFTLTISSLQPEDFATYYC |
| | 108VL10.Me | |
| 115 | LFR4 | FGQGTKVEIK |
| | 108VL10.Me | |
| 116 | HCDR1 | GYXFTHHWXH |
| | Formula | |
| 117 | HCDR2 | MIDASDXETRYXQKFXG |
| | Formula | |
| 118 | HCDR1 | GXXFXHHWIH |
| | Formula | |
| 119 | HCDR2 | MIDASDSETRLXXXXKX |
| | Formula | |

1. An antibody or antigen-binding fragment thereof, that binds to CD47, comprising: HCDR1, HCDR2 and HCDR3 of the VH as set forth in SEQ ID NO: 23, 22, 27, 47, 49, 8, 9, 10, 11, 12, 13, 14, 15, 16, 17, 18, 19, 20, 21, 24, 25, 26, 37, 38, 39, 40, 41, 42, 43, 44, 45, 46, 48, 50, 51, or 52; and LCDR1, LCDR2 and LCDR3 of the VL as set forth in SEQ ID NO: 7 or 36.

2. The antibody or antigen-binding fragment thereof of claim 1,
wherein the HCDR1 is defined according to AbM numbering system and HCDR2, HCDR3, LCDR1, LCDR2 and LCDR3 are defined according to Kabat numbering system.

3. (canceled)

4. The antibody or antigen-binding fragment thereof of claim 1, comprising:
HCDR1, HCDR2 and HCDR3 comprising the amino acid sequences of SEQ ID NOs: 57, 73, and 90, respectively; and LCDR1, LCDR2 and LCDR3 comprising the amino acid sequences of SEQ ID NOs: 93, 97, and 99, respectively;
HCDR1, HCDR2 and HCDR3 comprising the amino acid sequences of SEQ ID NOs: 57, 72, and 90, respectively; and LCDR1, LCDR2 and LCDR3 comprising the amino acid sequences of SEQ ID NOs: 93, 97, and 99, respectively; or,
HCDR1, HCDR2 and HCDR3 comprising the amino acid sequences of SEQ ID NOs: 57, 77, and 90, respectively; and LCDR1, LCDR2 and LCDR3 comprising the amino acid sequences of SEQ ID NOs: 93, 97, and 99, respectively; or
HCDR1 comprising the amino acid sequence of SEQ ID NO: 57, HCDR2 comprising the amino acid sequence of SEQ ID NO: 65, 66, 68, 69, 70, 71, 74, 75, or 76, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 56, HCDR2 comprising the amino acid sequence of SEQ ID NO: 65, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 55, HCDR2 comprising the amino acid sequence of SEQ ID NO: 62, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 54, HCDR2 comprising the amino acid sequence of SEQ ID NO: 62, 65, 66, or 67, HCDR3 comprising the amino acid sequence of SEQ ID NO: 90; and LCDR1, LCDR2 and LCDR3 comprising the amino acid sequences of SEQ ID NOs: 93, 97, and 99, respectively; or,
HCDR1 comprising the amino acid sequence of SEQ ID NO: 53, HCDR2 comprising the amino acid sequence of SEQ ID NO: 63 or 64, HCDR3 comprising the amino acid sequence of SEQ ID NO: 90; and LCDR1, LCDR2 and LCDR3 comprising the amino acid sequences of SEQ ID NOs: 93, 97, and 99, respectively.

5. (canceled)

6. The antibody or antigen-binding fragment thereof of claim 4, further comprising: four heavy chain framework regions (HFR1, HFR2, HFR3, and HFR4) comprising the amino acid sequences of SEQ ID NOs: 100, 101, 102 and 103, respectively; and/or, four light chain framework regions (LFR1, LFR2, LFR3, and LFR4) comprising the amino acid sequences of SEQ ID NOs: 108, 109, 110 and 111, respectively.

7. The antibody or antigen-binding fragment thereof of claim 4, comprising a heavy chain variable region (VH) and a light chain variable region (VL), wherein:
the VH comprises the sequence of SEQ ID NO: 23 or an amino acid sequence having at least 80% sequence identity thereto; and/or, the VL comprises the sequence of SEQ ID NO: 7 or an amino acid sequence having at least 80% sequence identity thereto; or
the VH comprises the sequence of SEQ ID NO: 22 or an amino acid sequence having at least 80% sequence identity thereto; and/or, the VL comprises the sequence of SEQ ID NO: 7 or an amino acid sequence having at least 80% sequence identity thereto; or
the VH comprises the sequence of SEQ ID NO: 27 or an amino acid sequence having at least 80% sequence identity thereto; and/or, the VL comprises the sequence of SEQ ID NO: 7 or an amino acid sequence having at least 80% sequence identity thereto; or
the VH comprises the sequence of SEQ ID NOs: 8, 9, 10, 11, 12, 13, 14, 15, 16, 17, 18, 19, 20, 21, 24, 25, or 26 or an amino acid sequence having at least 80% sequence identity thereto; and/or, the VL comprises the sequence of SEQ ID NO: 7 or an amino acid sequence having at least 80% sequence identity thereto.

8. (canceled)

9. (canceled)

10. The antibody or antigen-binding fragment thereof of claim 1, comprising:
HCDR1, HCDR2 and HCDR3 comprising the amino acid sequences of SEQ ID NOs: 58, 84, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 86, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 87, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 88, and 90, respectively; and LCDR1, LCDR2 and LCDR3 comprising the amino acid sequences of SEQ ID NOs: 95, 98, and 99, respectively; or,
HCDR1, HCDR2 and HCDR3 comprising the amino acid sequences of SEQ ID NOs: 58, 89, and 90, respectively; and LCDR1, LCDR2 and LCDR3 comprising the amino acid sequences of SEQ ID NOs: 95, 98, and 99, respectively; or
HCDR1 comprising the amino acid sequence of SEQ ID NO: 58; HCDR2 comprising the amino acid sequence of SEQ ID NO: 61, 62, 81, 82, 83, or 85; HCDR3 comprising the amino acid sequence of SEQ ID NO: 90; and LCDR1, LCDR2 and LCDR3 comprising the amino acid sequences of SEQ ID NOs: 95, 98, and 99, respectively; or
HCDR1 comprising the amino acid sequence of SEO ID NO: 53; HCDR2 comprising the amino acid sequence of SEQ ID NO: 80; HCDR3 comprising the amino acid sequence of SEQ ID NO: 90; and LCDR1, LCDR2 and LCDR3 comprising the amino acid sequences of SEQ ID NOs: 95, 98, and 99, respectively.

11. (canceled)

12. The antibody or antigen-binding fragment thereof of claim 10, further comprising: four heavy chain framework regions (HFR1, HFR2, HFR3, HFR4) comprising the amino acid sequences of SEQ ID NOs: 104, 105, 106 and 107, respectively; and/or, four light chain framework regions (LFR1, LFR2, LFR3, LFR4) comprising the amino acid sequences of SEQ ID NOs: 112, 113, 114 and 115, respectively.

13. The antibody or antigen-binding fragment thereof of claim 10, comprising a heavy chain variable region (VH) and a light chain variable region (VL), wherein:
the VH comprises the sequence of SEQ ID NO: 47 or an amino acid sequence having at least 80% sequence identity thereto; and/or, the VL comprises the sequence of SEQ ID NO: 36 or an amino acid sequence having at least 80% sequence identity thereto; or
the VH comprises the sequence of SEQ ID NO: 49 or an amino acid sequence having at least 80% sequence identity thereto; and/or, the VL comprises the sequence of SEQ ID NO: 36 or an amino acid sequence having at least 80% sequence identity thereto; or
the VH comprises the sequence of SEQ ID NO: 50 or an amino acid sequence having at least 80% sequence identity thereto; and/or, the VL comprises the sequence of SEQ ID NO: 36 or an amino acid sequence having at least 80% sequence identity thereto; or
the VH comprises the sequence of SEQ ID NO: 51 or an amino acid sequence having at least 80% sequence identity thereto; and/or, the VL comprises the sequence of SEQ ID NO: 36 or an amino acid sequence having at least 80% sequence identity thereto; or
the VH comprises the sequence of SEQ ID NO: 52 or an amino acid sequence having at least 80% sequence identity thereto; and/or, the VL comprises the sequence of SEQ ID NO: 36 or an amino acid sequence having at least 80% sequence identity thereto; or
the VH comprises the sequence of SEQ ID NO: 37, 42, 43, 44, 45, 46, or 48 or an amino acid sequence having at least 80% sequence identity thereto; and/or, the VL comprises the sequence of SEQ ID NO: 36 or an amino acid sequence having at least 80% sequence identity thereto.

14. The antibody or antigen-binding fragment thereof of claim 1, further comprising: a heavy chain constant region (CH) comprising an amino acid sequence derived from a human immunoglobulin heavy chain constant region.

15. (canceled)

16. The antibody or antigen-binding fragment thereof of claim 1, wherein the antibody or antigen-binding fragment thereof is selected from the group consisting of scFv, Fab, Fab′, (Fab′)₂, a Fv fragment, a diabody, a bispecific antibody, a multispecific antibody, and a humanized antibody.

17. An immunoconjugate, comprising the antibody or antigen-binding fragment thereof of claim 1 and an effector molecule.

18. The immunoconjugate of claim 17, wherein the effector molecule is a therapeutic agent selected from the group consisting of a drug, a toxin, a radioisotope, a protein, a peptide, and a nucleic acid.

19. A bispecific or multispecific antibody, comprising the antibody or antigen-binding fragment thereof of claim 1.

20. An isolated nucleic acid molecule, comprising a nucleotide sequence encoding the antibody or antigen-binding fragment thereof of claim 1.

21. A vector, comprising the isolated nucleic acid molecule of claim 20.

22. A host cell, comprising the isolated nucleic acid molecule of claim 20.

23. (canceled)

24. A pharmaceutical composition, comprising the antibody or antigen-binding fragment thereof of claim 1, and a pharmaceutically acceptable carrier and/or excipient.

25. The pharmaceutical composition of claim 24, which further comprises an additional therapeutic agent.

26.-29. (canceled)

30. A method of treating a cancer or proliferative disorder in a subject, wherein the method comprising administering to a subject in need thereof an effective amount of the antibody or antigen-binding fragment thereof of claim 1.

31. (canceled)

32. The method of claim 30, wherein the cancer or proliferative disorder is a hematological cancer, e.g., leukemia, lymphoma or myeloma or a solid tumor.
